(19) United States
(12) Patent Application Publication
(10) Pub. No.: US 2026/0070911 A1
(43) Pub. Date: Mar. 12, 2026

(54) INHIBITORS OF TAM RECEPTORS

(71) Applicant: National Health Research Institutes, Miaoli County (TW)

(72) Inventors: Hsing-Pang Hsieh, Miaoli County (TW); Wan-Ching Yen, Miaoli County (TW); Su-Ying Wu, Miaoli County (TW); Mu-Chun Li, Miaoli County (TW); Wen-Hsing Lin, Miaoli County (TW); Yi-Yu Ke, Hsinchu County (TW)

(21) Appl. No.: 19/104,616

(22) PCT Filed: Aug. 18, 2023
(86) PCT No.: PCT/US2023/030571

§ 371 Date: Feb. 18, 2025

Related U.S. Application Data
(60) Provisional application No. 63/399,404, filed on Aug. 19, 2022.

(51) Int. Cl. C07D 487/04; A61K 31/519; A61K 31/5377; C07B 59/00; C07D 491/048; C07D 495/04

(52) U.S. Cl. CPC C07D 487/04; A61K 31/519; A61K 31/5377; C07B 59/002; C07D 491/048; C07D 495/04

(57) ABSTRACT

Disclosed are compounds of formula (I):

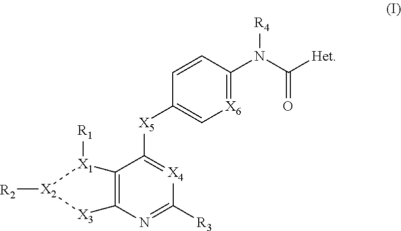

Variables R₁-R₄, X₁-X₆, and Het are defined herein. Also disclosed are a pharmaceutical composition containing such a compound and methods of using the compound to treating disorders associated with TAM.

### CROSS-REFERENCE TO RELATED APPLICATION

This application claims the benefit of priority based on U.S. Provisional Application No. 63/399,404, Aug. 19, 2022, the content and disclosure of which are incorporated herein by reference in its entirety.

### BACKGROUND

TAM receptors include TYRO3, AXL, and MERTK. They are a well-studied family of receptor tyrosine kinases associated with immune system diseases, kidney diseases, circulatory system diseases, and cancer. See Graham et al., Nature Reviews 14, 769 (2014) and Paplino et al., Cancers 8, 97 (2016).

In a cancer patient, TAM receptors (e.g., AXL and MERTK) have a dual regulatory role. They control not only the initiation and progression of tumor cells but also anti-tumor responses of diverse immune cells. See id. Such responses include tumor-associated macrophages (e.g., M1 and M2 phenotypes) activated by MERTK, a TAM receptor. See Genard, Frontiers in Immunology 8, 828 (2017). While M2 phenotype plays a central role in tumor progression, metastasis, and recurrence after treatment, M1 phenotype is responsible for antitumor immune responses. See id.

It has been reported that (i) AXL overexpression decreases M1 phenotype macrophages, (ii) MERTK overexpression decreases M1 phenotype macrophages and increases M2 phenotype macrophages, (iii) selective inhibition of AXL leads to drug-resistance by upregulation of MERTK, and (iv) inhibition of both AXL and MERTK enhances anti-tumor efficacy and anti-tumor immune responses. See Linger et al., Oncogene, 32, 3420-3431 (2013) and McDaniel et al., Molecular Cancer Therapeutics, 17 (11), 2297-2308 (2018).

Currently, TAM inhibitors have not been marketed for treating cancer. A very limited number of compounds have been studied at preclinical stage. Their efficacy remains unclear.

There is a need to develop inhibitors of AXL, MERTK, or both for treatment of TAM-associated disorders including cancer, immune system diseases, kidney diseases, and circulatory system diseases.

### SUMMARY

The present invention is based on an unexpected discovery that certain heteroaryl compounds effectively inhibit AXL, MERTK, or both, suitable for treating cancer, immune system diseases, kidney diseases, and circulatory system diseases.

In one aspect, this invention relates to compounds of formula (I):

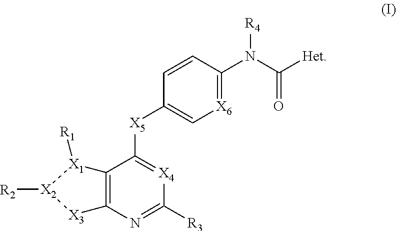

In this formula, each of R₁ and R₂, independently, is H, deuterium (D), halo, C₁₋₆ alkyl, C₃₋₆ cycloalkyl, C₁₋₆ heterocycloalkyl, aryl, heteroaryl, or deleted;
R₃ is H, deuterium, halo, C₁₋₆ alkyl, C₃₋₆ cycloalkyl, C₁₋₆ heterocycloalkyl, aryl, heteroaryl, or NR₃ₐR_{3b}, in which each of R₃ₐ and R_{3b}, independently, is H, deuterium, C₁₋₆ alkyl, or aryl;
R₄ is H, deuterium, C₁₋₆ alkyl, C₃₋₆ cycloalkyl, or C₁₋₆ heterocycloalkyl;
X₁ is C, CH, CD, or S;
X₂ is C or N;
X₃ is O, CH, S, or NH;
at least one of X₁, X₂, and X₃ is O, S, N, or NH;
X₄ is N, CH, or CH(CN);
X₅ is O or NH;
X₆ is N or CH;
Het is selected from the group consisting of:

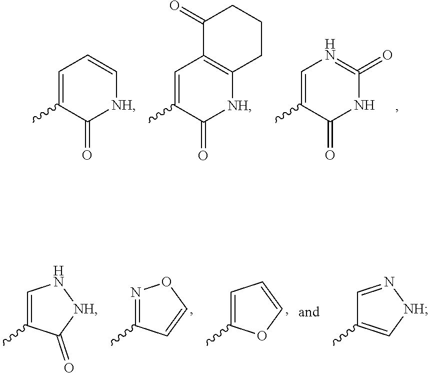

one of the two [symbol] is a single bond and the other is a double bond; and
each of C₁₋₆ alkyl, C₃₋₆ cycloalkyl, C₁₋₆ heterocycloalkyl, aryl, heteroaryl, and Het is optionally substituted with one or more of the chemical groups consisting of deuterium, hydroxyl, halo, nitro, cyano, amino, C₁₋₆ acylamino, C₁₋₆ alkylamino, C₁₋₆ alkyl, C₁₋₆ aminoalkyl, C₁₋₆ alkoxyl, C₁₋₆ alkylcarbonyl, C₃₋₁₀ cycloalkyl, C₁₋₆ heterocycloalkyl, aralkyl, aryl, and heteroaryl. The substituents and their subsequent occurrences can be further substituted with the chemical groups described above.

Preferably, the above-described compounds have one or more of the following features:
(i) R₁ is H or phenyl;
(ii) R₂ is H, C₁₋₆ alkyl, or heteroaryl;
(iii) R₃ is H, methyl, phenyl, pyridinyl, methylamino, or phenylamino;
(iv) R₄ is H;
(v) each of X₁ and X₂ is C;
(vi) the [symbol] between X₁ and X₂ is a double bond;
(vii) X₃ is O or NH;
(viii) X₄ is N;
(ix) X₅ is O;
(x) X₆ is CH; and
(xi) Het is

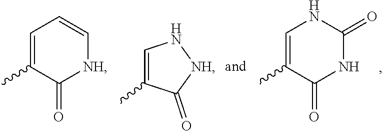

optionally substituted with one or more of methyl, ethoxy, phenyl, 3-fluorophenyl, 4-fluorophenyl, 2-methyl-4-fluorophenyl, 2-fluoropyridin-3-yl, and butylenecarbonyl.

A subset of the compounds of formula (I) includes the compounds of formula (II)

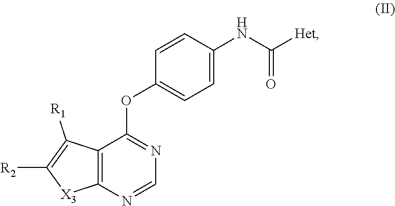

in which each of R₁, R₂, X₃ and Het is defined above.

Some preferred compounds of formula (II) have one or more of the following features: R₁ is phenyl, R₂ is H or heteroaryl, X₃ is O or NH, Het is

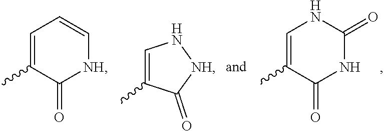

each of phenyl and heteroaryl is optionally substituted with one or more of the chemical groups consisting of amino, C₁₋₆ acylamino, C₁₋₆ alkylamino, C₁₋₆ alkyl, and C₁₋₆ aminoalkyl, and Het is optionally substituted with one or more of C₁₋₆ alkyl and phenyl, phenyl being optionally substituted with hydroxyl, halo, amino, C₁₋₆ alkyl, or C₁₋₆ alkoxy.

Table 1 below shows 178 exemplary compounds of the present invention, i.e., Compounds 1-178, together with their structures and mass spectrometry (m/z) data.

TABLE 1

| Com- | | Mass |
|---|---|---|
| pound | Structure | [M + H]⁺ |
| 1 | 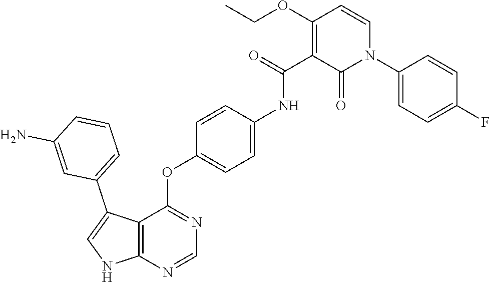 [SMILES: CCOc1ccn(-c2ccc(F)cc2)c(=O)c1C(=O)Nc1ccc(Oc2ncnc3[nH]cc(-c4cccc(N)c4)c23)cc1] | 577 |
| 2 | 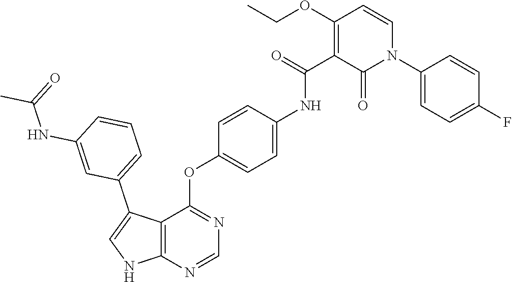 [SMILES: CCOc1ccn(-c2ccc(F)cc2)c(=O)c1C(=O)Nc1ccc(Oc2ncnc3[nH]cc(-c4cccc(NC(C)=O)c4)c23)cc1] | 619 |
| 3 | 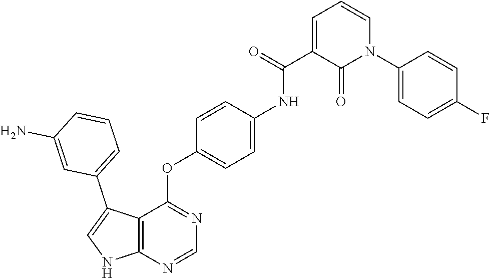 [SMILES: Nc1cccc(-c2c[nH]c3ncnc(Oc4ccc(NC(=O)c5cccn(-c6ccc(F)cc6)c5=O)cc4)c23)c1] | 533 |
| 4 | 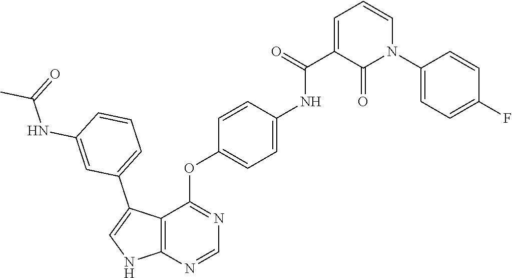 [SMILES: CC(=O)Nc1cccc(-c2c[nH]c3ncnc(Oc4ccc(NC(=O)c5cccn(-c6ccc(F)cc6)c5=O)cc4)c23)c1] | 575 |
| 5 | 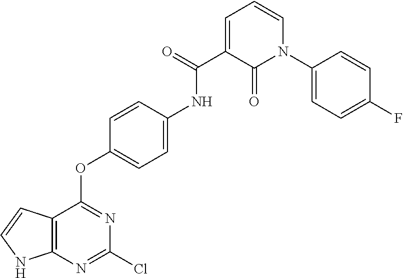 [SMILES: O=C(Nc1ccc(Oc2nc(Cl)nc3[nH]ccc23)cc1)c1cccn(-c2ccc(F)cc2)c1=O] | 476 |
| 6 | 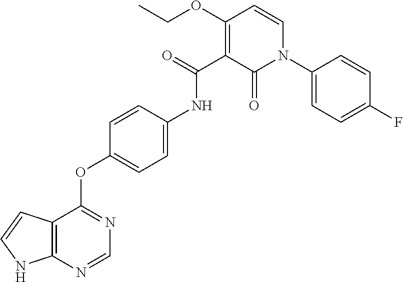 [SMILES: CCOc1ccn(-c2ccc(F)cc2)c(=O)c1C(=O)Nc1ccc(Oc2ncnc3[nH]ccc23)cc1] | 486 |
| 7 | 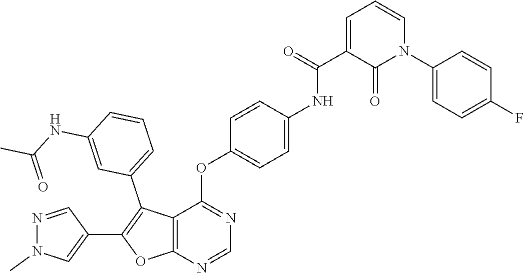 [SMILES: CC(=O)Nc1cccc(-c2c(-c3cnn(C)c3)oc3ncnc(Oc4ccc(NC(=O)c5cccn(-c6ccc(F)cc6)c5=O)cc4)c23)c1] | 656 |
| 8 | 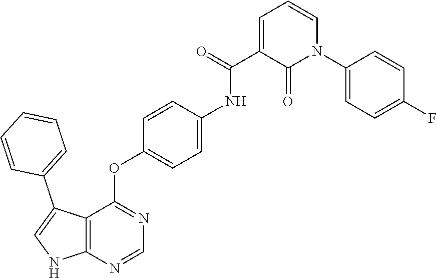 [SMILES: O=C(Nc1ccc(Oc2ncnc3[nH]cc(-c4ccccc4)c23)cc1)c1cccn(-c2ccc(F)cc2)c1=O] | 518 |
| 9 | 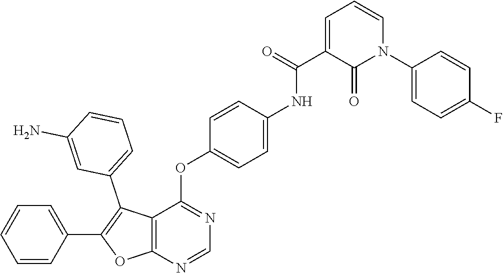 [SMILES: Nc1cccc(-c2c(-c3ccccc3)oc3ncnc(Oc4ccc(NC(=O)c5cccn(-c6ccc(F)cc6)c5=O)cc4)c23)c1] | 610 |
| 10 | 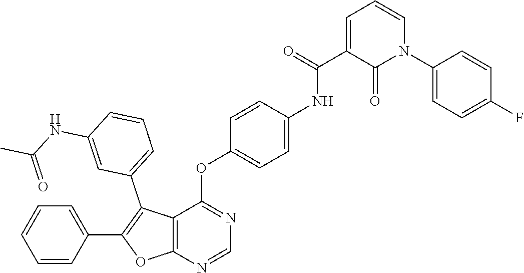 [SMILES: CC(=O)Nc1cccc(-c2c(-c3ccccc3)oc3ncnc(Oc4ccc(NC(=O)c5cccn(-c6ccc(F)cc6)c5=O)cc4)c23)c1] | 664 |
| 11 | 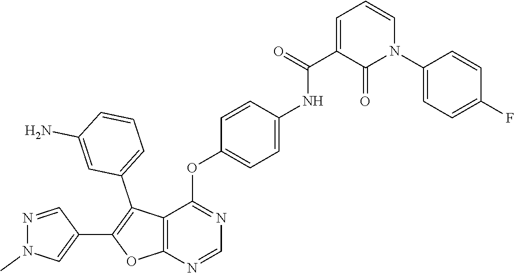 [SMILES: Cn1cc(-c2oc3ncnc(Oc4ccc(NC(=O)c5cccn(-c6ccc(F)cc6)c5=O)cc4)c3c2-c2cccc(N)c2)cn1] | 614 |
| 12 | 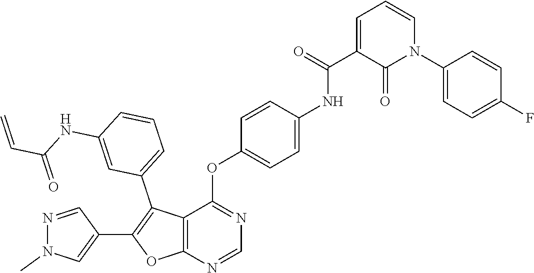 [SMILES: C=CC(=O)Nc1cccc(-c2c(-c3cnn(C)c3)oc3ncnc(Oc4ccc(NC(=O)c5cccn(-c6ccc(F)cc6)c5=O)cc4)c23)c1] | 668 |
| 13 | 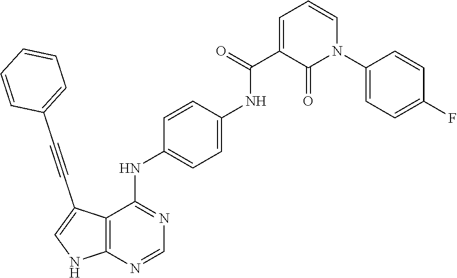 [SMILES: O=C(Nc1ccc(Nc2ncnc3[nH]cc(C#Cc4ccccc4)c23)cc1)c1cccn(-c2ccc(F)cc2)c1=O] | 541 |
| 14 | 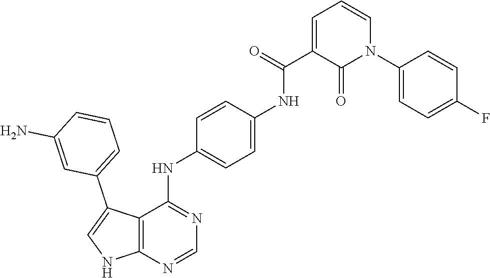 [SMILES: Nc1cccc(-c2c[nH]c3ncnc(Nc4ccc(NC(=O)c5cccn(-c6ccc(F)cc6)c5=O)cc4)c23)c1] | 532 |
| 15 | 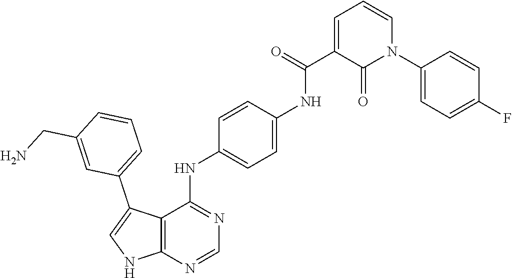 [SMILES: NCc1cccc(-c2c[nH]c3ncnc(Nc4ccc(NC(=O)c5cccn(-c6ccc(F)cc6)c5=O)cc4)c23)c1] | 547 |
| 16 | 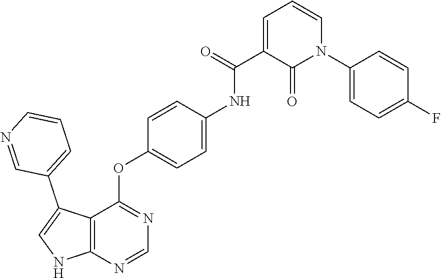 [SMILES: O=C(Nc1ccc(Oc2ncnc3[nH]cc(-c4cccnc4)c23)cc1)c1cccn(-c2ccc(F)cc2)c1=O] | 519 |
| 17 | 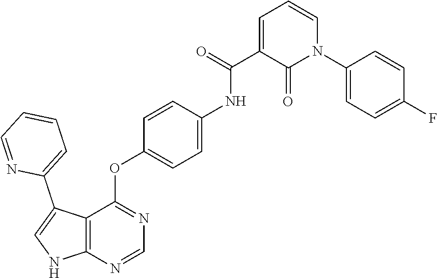 [SMILES: O=C(Nc1ccc(Oc2ncnc3[nH]cc(-c4ccccn4)c23)cc1)c1cccn(-c2ccc(F)cc2)c1=O] | 519 |
| 18 | 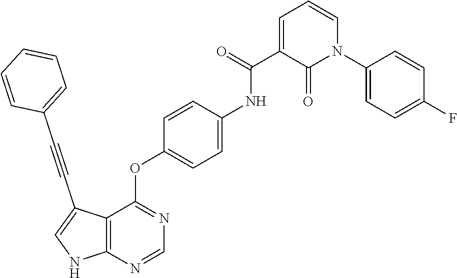 [SMILES: O=C(Nc1ccc(Oc2ncnc3[nH]cc(C#Cc4ccccc4)c23)cc1)c1cccn(-c2ccc(F)cc2)c1=O] | 542 |
| 19 | 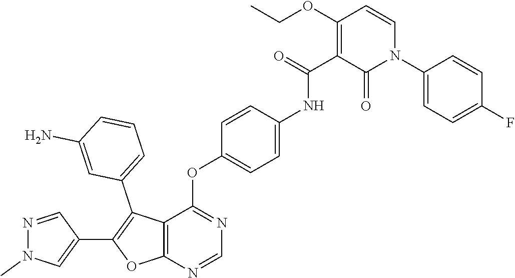 [SMILES: CCOc1ccn(-c2ccc(F)cc2)c(=O)c1C(=O)Nc1ccc(Oc2ncnc3oc(-c4cnn(C)c4)c(-c4cccc(N)c4)c23)cc1] | 658 |
| 20 | 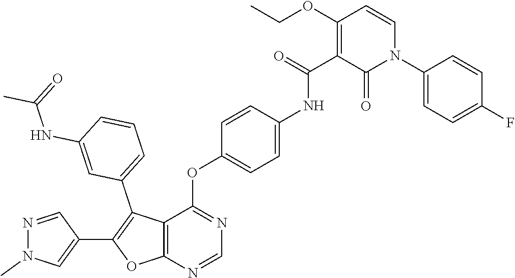 [SMILES: CCOc1ccn(-c2ccc(F)cc2)c(=O)c1C(=O)Nc1ccc(Oc2ncnc3oc(-c4cnn(C)c4)c(-c4cccc(NC(C)=O)c4)c23)cc1] | 670 |
| 21 | 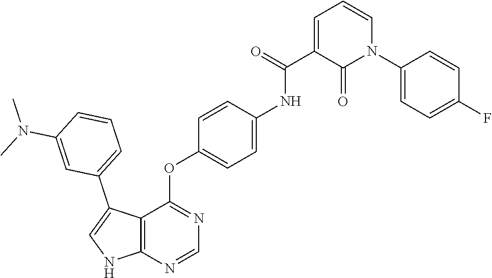 [SMILES: CN(C)c1cccc(-c2c[nH]c3ncnc(Oc4ccc(NC(=O)c5cccn(-c6ccc(F)cc6)c5=O)cc4)c23)c1] | 561 |
| 22 | 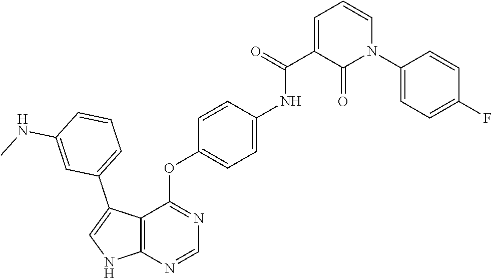 [SMILES: CNc1cccc(-c2c[nH]c3ncnc(Oc4ccc(NC(=O)c5cccn(-c6ccc(F)cc6)c5=O)cc4)c23)c1] | 547 |
| 23 | 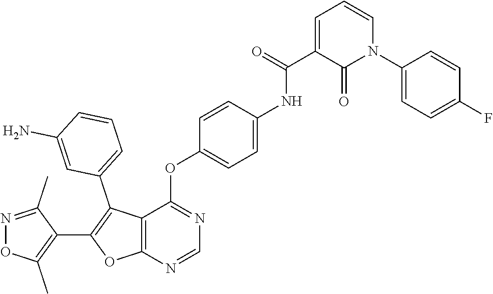 [SMILES: Cc1noc(C)c1-c1oc2ncnc(Oc3ccc(NC(=O)c4cccn(-c5ccc(F)cc5)c4=O)cc3)c2c1-c1cccc(N)c1] | 629 |
| 24 | 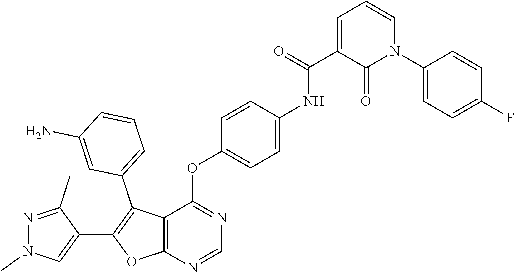 [SMILES: Cc1nn(C)cc1-c1oc2ncnc(Oc3ccc(NC(=O)c4cccn(-c5ccc(F)cc5)c4=O)cc3)c2c1-c1cccc(N)c1] | 628 |
| 25 | 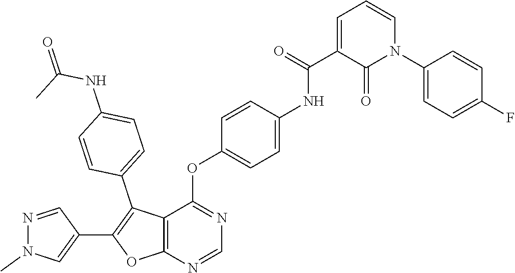 [SMILES: CC(=O)Nc1ccc(-c2c(-c3cnn(C)c3)oc3ncnc(Oc4ccc(NC(=O)c5cccn(-c6ccc(F)cc6)c5=O)cc4)c23)cc1] | 656 |
| 26 | 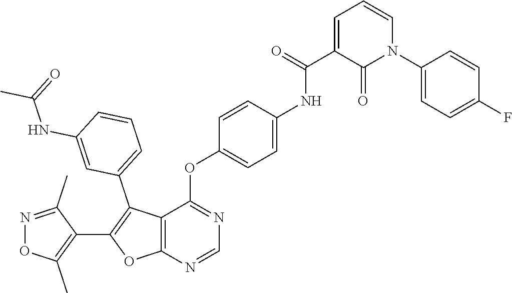 [SMILES: CC(=O)Nc1cccc(-c2c(-c3c(C)noc3C)oc3ncnc(Oc4ccc(NC(=O)c5cccn(-c6ccc(F)cc6)c5=O)cc4)c23)c1] | 671 |
| 27 | 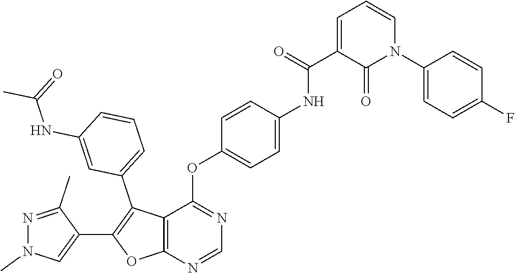 [SMILES: CC(=O)Nc1cccc(-c2c(-c3cn(C)nc3C)oc3ncnc(Oc4ccc(NC(=O)c5cccn(-c6ccc(F)cc6)c5=O)cc4)c23)c1] | 670 |
| 28 | 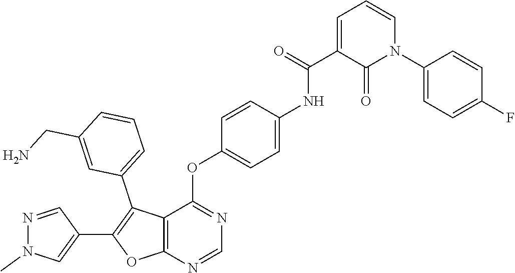 [SMILES: Cn1cc(-c2oc3ncnc(Oc4ccc(NC(=O)c5cccn(-c6ccc(F)cc6)c5=O)cc4)c3c2-c2cccc(CN)c2)cn1] | 628 |
| 29 | 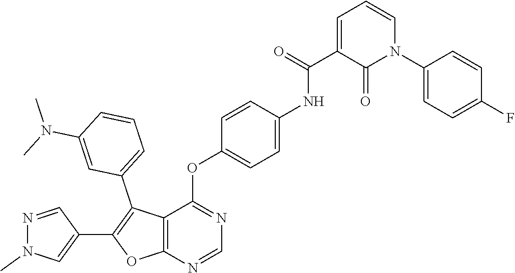 [SMILES: CN(C)c1cccc(-c2c(-c3cnn(C)c3)oc3ncnc(Oc4ccc(NC(=O)c5cccn(-c6ccc(F)cc6)c5=O)cc4)c23)c1] | 642 |
| 30 | 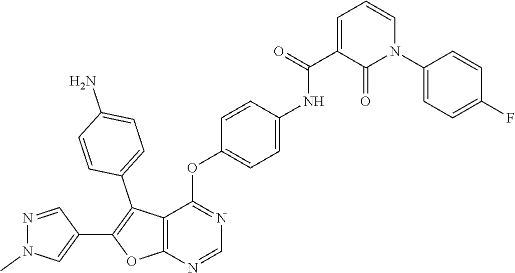 [SMILES: Cn1cc(-c2oc3ncnc(Oc4ccc(NC(=O)c5cccn(-c6ccc(F)cc6)c5=O)cc4)c3c2-c2ccc(N)cc2)cn1] | 614 |
| 31 | 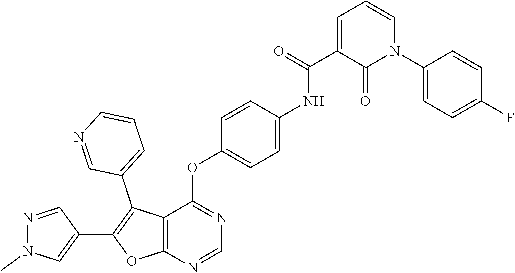 [SMILES: Cn1cc(-c2oc3ncnc(Oc4ccc(NC(=O)c5cccn(-c6ccc(F)cc6)c5=O)cc4)c3c2-c2cccnc2)cn1] | 600 |
| 32 | 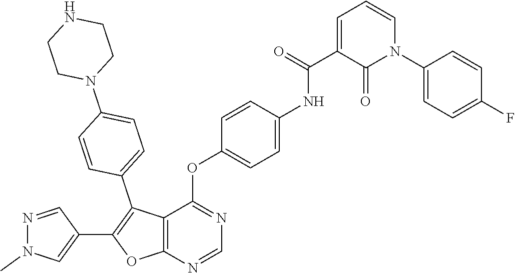 [SMILES: Cn1cc(-c2oc3ncnc(Oc4ccc(NC(=O)c5cccn(-c6ccc(F)cc6)c5=O)cc4)c3c2-c2ccc(N3CCNCC3)cc2)cn1] | 683 |
| 33 | 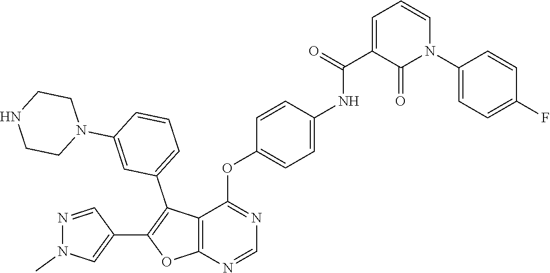 [SMILES: Cn1cc(-c2oc3ncnc(Oc4ccc(NC(=O)c5cccn(-c6ccc(F)cc6)c5=O)cc4)c3c2-c2cccc(N3CCNCC3)c2)cn1] | 683 |
| 34 | 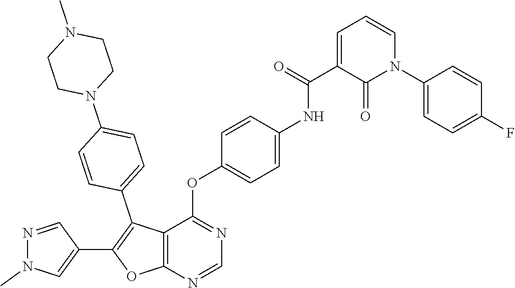 [SMILES: CN1CCN(c2ccc(-c3c(-c4cnn(C)c4)oc4ncnc(Oc5ccc(NC(=O)c6cccn(-c7ccc(F)cc7)c6=O)cc5)c34)cc2)CC1] | 697 |
| 35 | 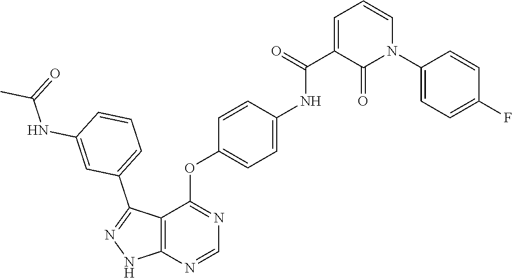 [SMILES: CC(=O)Nc1cccc(-c2n[nH]c3ncnc(Oc4ccc(NC(=O)c5cccn(-c6ccc(F)cc6)c5=O)cc4)c23)c1] | 576 |
| 36 | 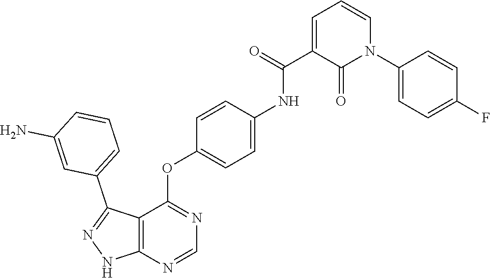 [SMILES: Nc1cccc(-c2n[nH]c3ncnc(Oc4ccc(NC(=O)c5cccn(-c6ccc(F)cc6)c5=O)cc4)c23)c1] | 534 |
| 37 | 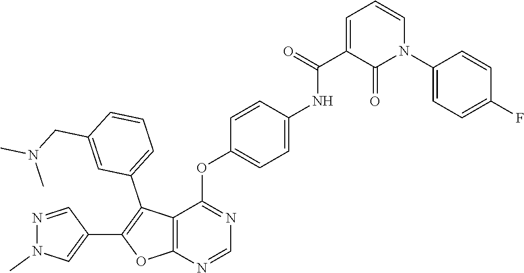 [SMILES: CN(C)Cc1cccc(-c2c(-c3cnn(C)c3)oc3ncnc(Oc4ccc(NC(=O)c5cccn(-c6ccc(F)cc6)c5=O)cc4)c23)c1] | 656 |
| 38 | 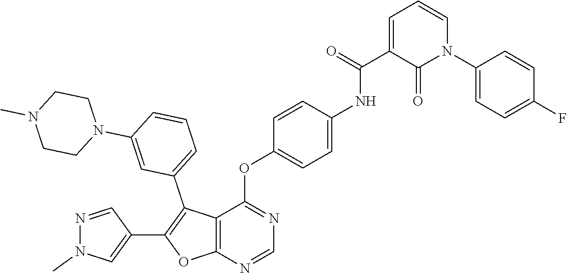 [SMILES: CN1CCN(c2cccc(-c3c(-c4cnn(C)c4)oc4ncnc(Oc5ccc(NC(=O)c6cccn(-c7ccc(F)cc7)c6=O)cc5)c34)c2)CC1] | 697 |
| 39 | 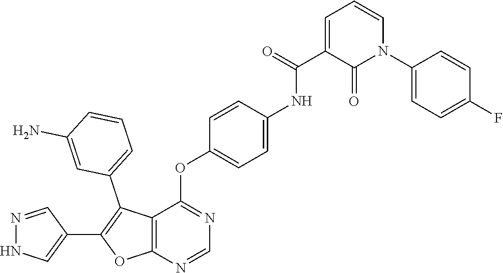 [SMILES: Nc1cccc(-c2c(-c3cn[nH]c3)oc3ncnc(Oc4ccc(NC(=O)c5cccn(-c6ccc(F)cc6)c5=O)cc4)c23)c1] | 600 |
| 40 | 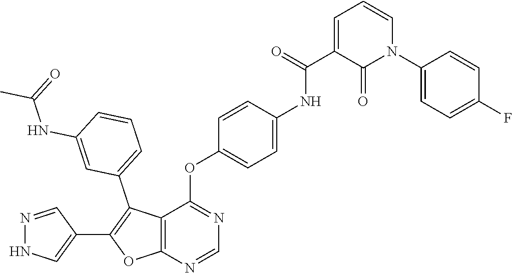 [SMILES: CC(=O)Nc1cccc(-c2c(-c3cn[nH]c3)oc3ncnc(Oc4ccc(NC(=O)c5cccn(-c6ccc(F)cc6)c5=O)cc4)c23)c1] | 642 |
| 41 | 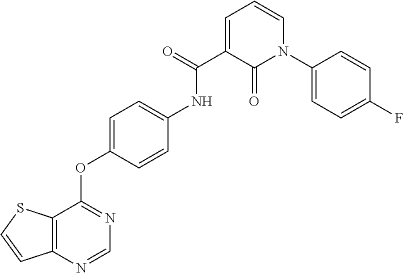 [SMILES: O=C(Nc1ccc(Oc2ncnc3ccsc23)cc1)c1cccn(-c2ccc(F)cc2)c1=O] | 459 |
| 42 | 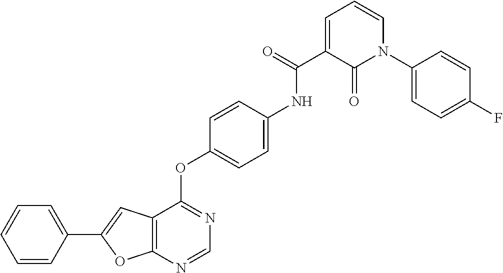 [SMILES: O=C(Nc1ccc(Oc2ncnc3oc(-c4ccccc4)cc23)cc1)c1cccn(-c2ccc(F)cc2)c1=O] | 519 |
| 43 | 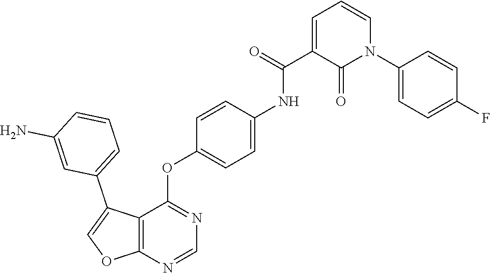 [SMILES: Nc1cccc(-c2coc3ncnc(Oc4ccc(NC(=O)c5cccn(-c6ccc(F)cc6)c5=O)cc4)c23)c1] | 534 |
| 44 | 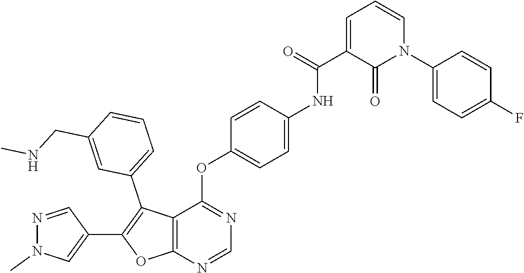 [SMILES: CNCc1cccc(-c2c(-c3cnn(C)c3)oc3ncnc(Oc4ccc(NC(=O)c5cccn(-c6ccc(F)cc6)c5=O)cc4)c23)c1] | 642 |
| 45 | 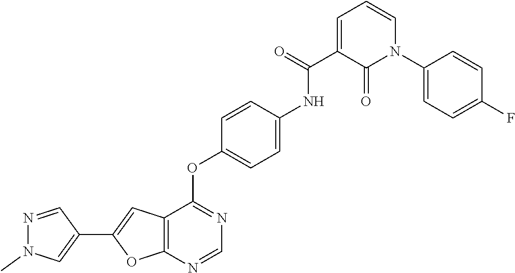 [SMILES: Cn1cc(-c2cc3c(Oc4ccc(NC(=O)c5cccn(-c6ccc(F)cc6)c5=O)cc4)ncnc3o2)cn1] | 523 |
| 46 | 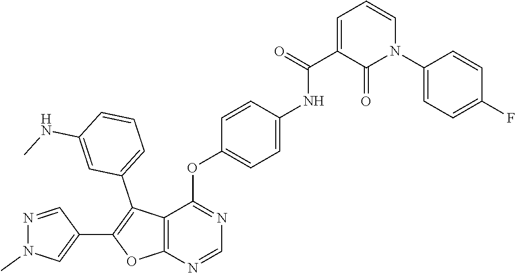 [SMILES: CNc1cccc(-c2c(-c3cnn(C)c3)oc3ncnc(Oc4ccc(NC(=O)c5cccn(-c6ccc(F)cc6)c5=O)cc4)c23)c1] | 628 |
| 47 | 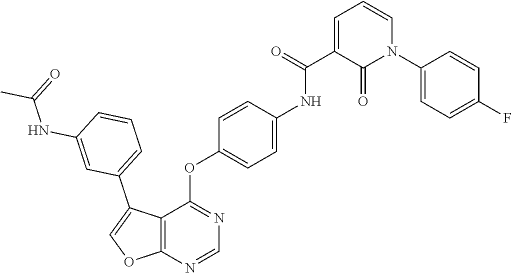 [SMILES: CC(=O)Nc1cccc(-c2coc3ncnc(Oc4ccc(NC(=O)c5cccn(-c6ccc(F)cc6)c5=O)cc4)c23)c1] | 576 |
| 48 | 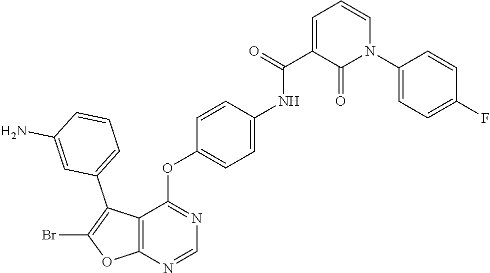 [SMILES: Nc1cccc(-c2c(Br)oc3ncnc(Oc4ccc(NC(=O)c5cccn(-c6ccc(F)cc6)c5=O)cc4)c23)c1] | 613 |
| 49 | 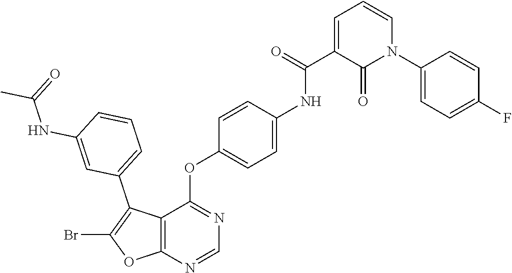 [SMILES: CC(=O)Nc1cccc(-c2c(Br)oc3ncnc(Oc4ccc(NC(=O)c5cccn(-c6ccc(F)cc6)c5=O)cc4)c23)c1] | 655 |
| 50 | 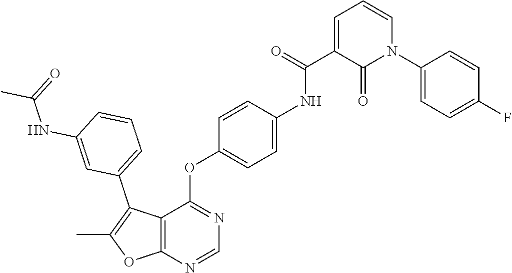 [SMILES: CC(=O)Nc1cccc(-c2c(C)oc3ncnc(Oc4ccc(NC(=O)c5cccn(-c6ccc(F)cc6)c5=O)cc4)c23)c1] | 590 |
| 51 | 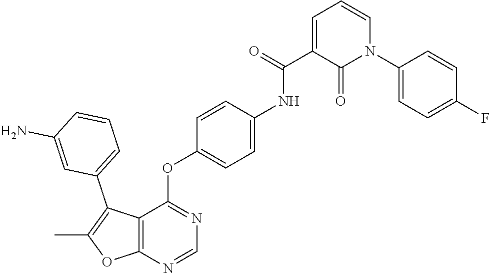 [SMILES: Cc1oc2ncnc(Oc3ccc(NC(=O)c4cccn(-c5ccc(F)cc5)c4=O)cc3)c2c1-c1cccc(N)c1] | 548 |
| 52 | 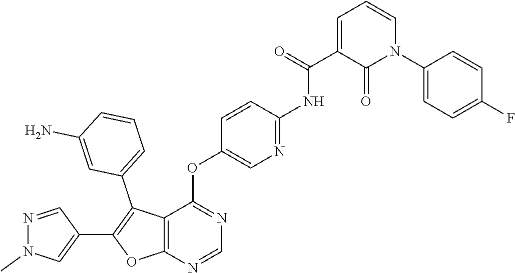 [SMILES: Cn1cc(-c2oc3ncnc(Oc4ccc(NC(=O)c5cccn(-c6ccc(F)cc6)c5=O)nc4)c3c2-c2cccc(N)c2)cn1] | 615 |
| 53 | 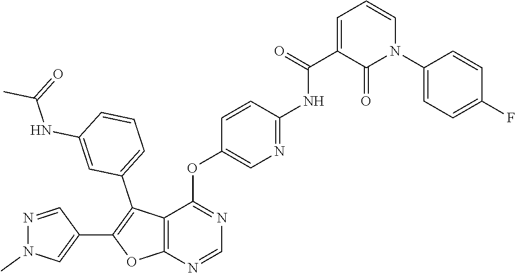 [SMILES: CC(=O)Nc1cccc(-c2c(-c3cnn(C)c3)oc3ncnc(Oc4ccc(NC(=O)c5cccn(-c6ccc(F)cc6)c5=O)nc4)c23)c1] | 657 |
| 54 | 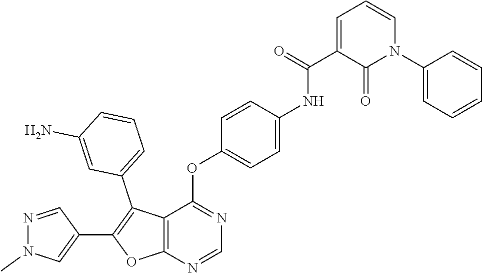 [SMILES: Cn1cc(-c2oc3ncnc(Oc4ccc(NC(=O)c5cccn(-c6ccccc6)c5=O)cc4)c3c2-c2cccc(N)c2)cn1] | 596 |
| 55 | 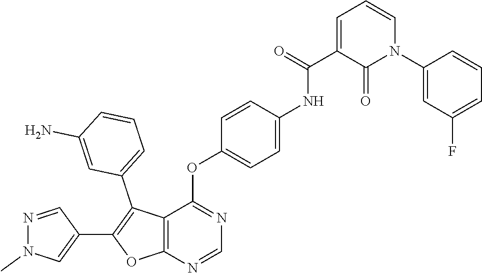 [SMILES: Cn1cc(-c2oc3ncnc(Oc4ccc(NC(=O)c5cccn(-c6cccc(F)c6)c5=O)cc4)c3c2-c2cccc(N)c2)cn1] | 614 |
| 56 | 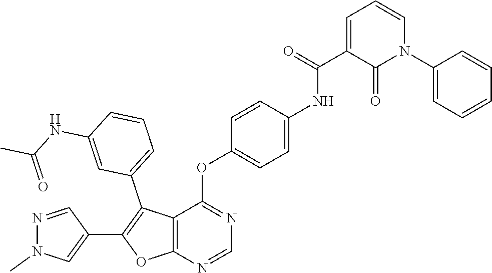 [SMILES: CC(=O)Nc1cccc(-c2c(-c3cnn(C)c3)oc3ncnc(Oc4ccc(NC(=O)c5cccn(-c6ccccc6)c5=O)cc4)c23)c1] | 638 |
| 57 | 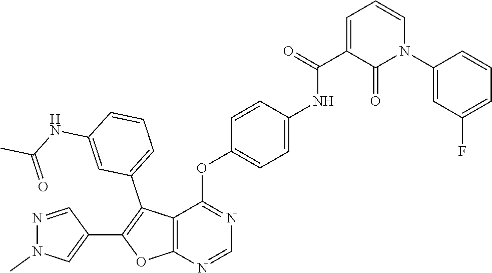 [SMILES: CC(=O)Nc1cccc(-c2c(-c3cnn(C)c3)oc3ncnc(Oc4ccc(NC(=O)c5cccn(-c6cccc(F)c6)c5=O)cc4)c23)c1] | 656 |
| 58 | 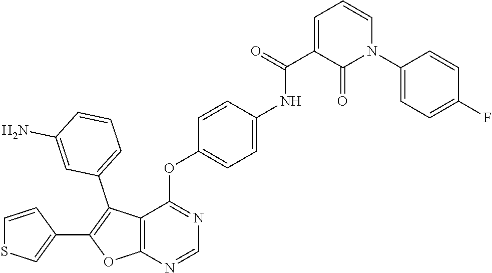 [SMILES: Nc1cccc(-c2c(-c3ccsc3)oc3ncnc(Oc4ccc(NC(=O)c5cccn(-c6ccc(F)cc6)c5=O)cc4)c23)c1] | 616 |
| 59 | 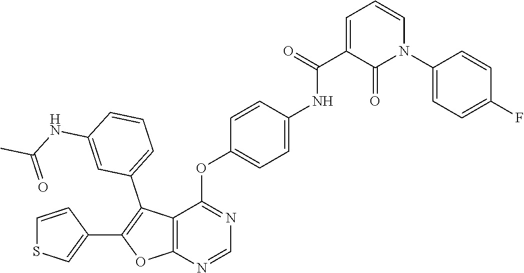 [SMILES: CC(=O)Nc1cccc(-c2c(-c3ccsc3)oc3ncnc(Oc4ccc(NC(=O)c5cccn(-c6ccc(F)cc6)c5=O)cc4)c23)c1] | 658 |
| 60 | 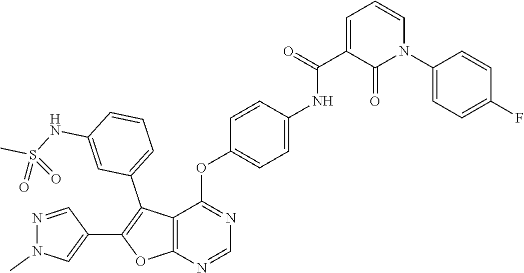 [SMILES: Cn1cc(-c2oc3ncnc(Oc4ccc(NC(=O)c5cccn(-c6ccc(F)cc6)c5=O)cc4)c3c2-c2cccc(NS(C)(=O)=O)c2)cn1] | 692 |
| 61 | 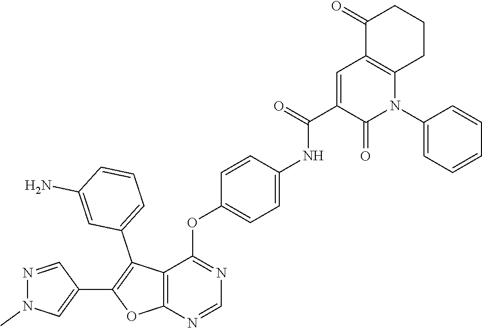 [SMILES: Cn1cc(-c2oc3ncnc(Oc4ccc(NC(=O)c5cc6c(n(-c7ccccc7)c5=O)CCCC6=O)cc4)c3c2-c2cccc(N)c2)cn1] | 664 |
| 62 | 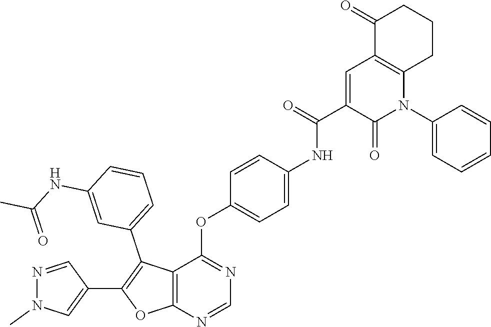 [SMILES: CC(=O)Nc1cccc(-c2c(-c3cnn(C)c3)oc3ncnc(Oc4ccc(NC(=O)c5cc6c(n(-c7ccccc7)c5=O)CCCC6=O)cc4)c23)c1] | 706 |
| 63 | 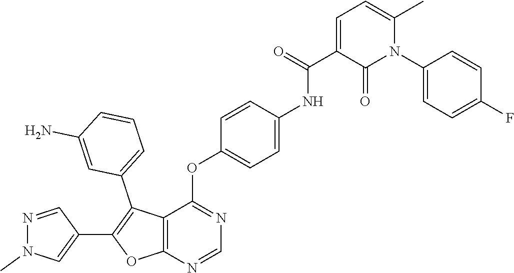 [SMILES: Cc1ccc(C(=O)Nc2ccc(Oc3ncnc4oc(-c5cnn(C)c5)c(-c5cccc(N)c5)c34)cc2)c(=O)n1-c1ccc(F)cc1] | 628 |
| 64 | 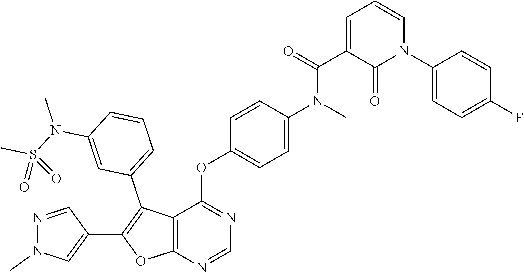 [SMILES: CN(C(=O)c1cccn(-c2ccc(F)cc2)c1=O)c1ccc(Oc2ncnc3oc(-c4cnn(C)c4)c(-c4cccc(N(C)S(C)(=O)=O)c4)c23)cc1] | 720 |
| 65 | 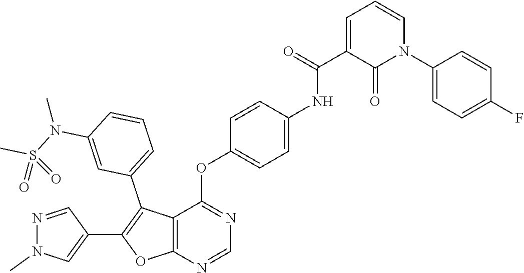 [SMILES: CN(c1cccc(-c2c(-c3cnn(C)c3)oc3ncnc(Oc4ccc(NC(=O)c5cccn(-c6ccc(F)cc6)c5=O)cc4)c23)c1)S(C)(=O)=O] | 706 |
| 66 | 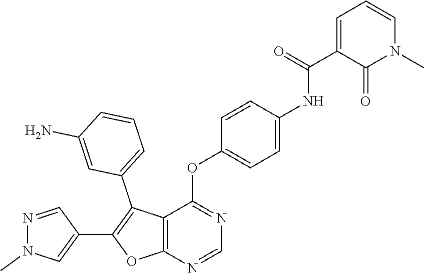 | 534 |
| 67 | 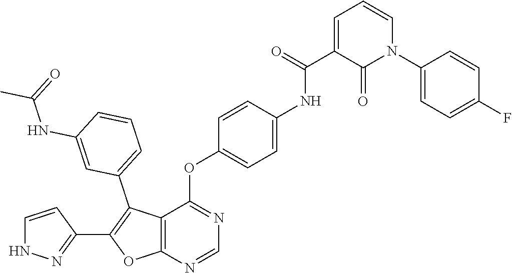 [SMILES: CC(=O)Nc1cccc(-c2c(-c3cc[nH]n3)oc3ncnc(Oc4ccc(NC(=O)c5cccn(-c6ccc(F)cc6)c5=O)cc4)c23)c1] | 642 |
| 68 | 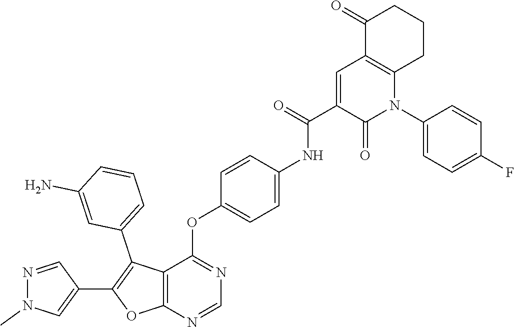 [SMILES: Cn1cc(-c2oc3ncnc(Oc4ccc(NC(=O)c5cc6c(n(-c7ccc(F)cc7)c5=O)CCCC6=O)cc4)c3c2-c2cccc(N)c2)cn1] | 682 |
| 69 | 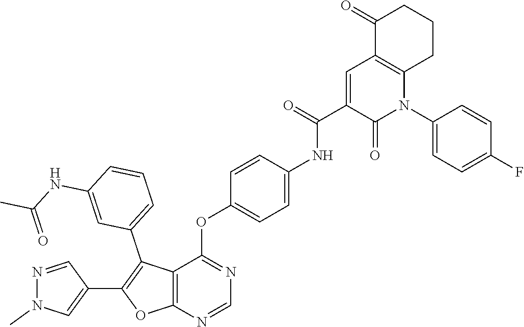 [SMILES: CC(=O)Nc1cccc(-c2c(-c3cnn(C)c3)oc3ncnc(Oc4ccc(NC(=O)c5cc6c(n(-c7ccc(F)cc7)c5=O)CCCC6=O)cc4)c23)c1] | 724 |
| 70 | 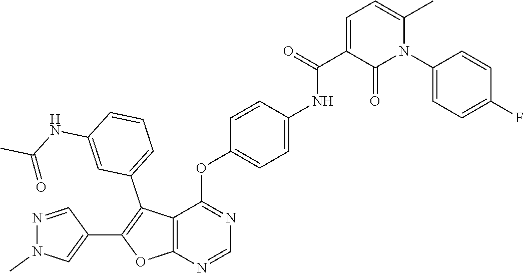 [SMILES: CC(=O)Nc1cccc(-c2c(-c3cnn(C)c3)oc3ncnc(Oc4ccc(NC(=O)c5ccc(C)n(-c6ccc(F)cc6)c5=O)cc4)c23)c1] | 670 |
| 71 | 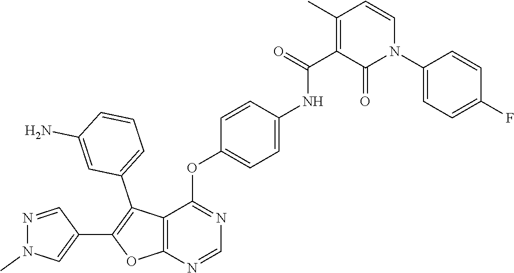 [SMILES: Cc1ccn(-c2ccc(F)cc2)c(=O)c1C(=O)Nc1ccc(Oc2ncnc3oc(-c4cnn(C)c4)c(-c4cccc(N)c4)c23)cc1] | 628 |
| 72 | 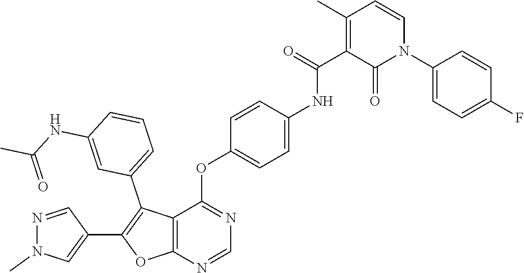 [SMILES: CC(=O)Nc1cccc(-c2c(-c3cnn(C)c3)oc3ncnc(Oc4ccc(NC(=O)c5c(C)ccn(-c6ccc(F)cc6)c5=O)cc4)c23)c1] | 670 |
| 73 | 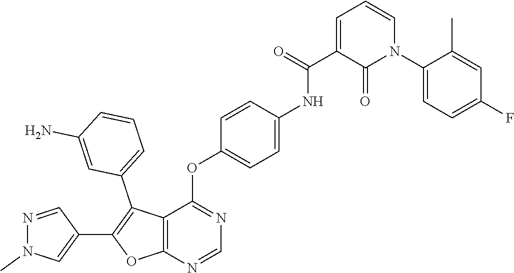 [SMILES: Cc1cc(F)ccc1-n1cccc(C(=O)Nc2ccc(Oc3ncnc4oc(-c5cnn(C)c5)c(-c5cccc(N)c5)c34)cc2)c1=O] | 628 |
| 74 | 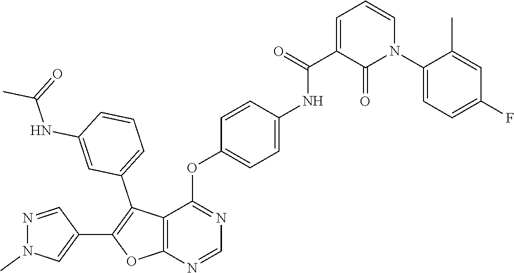 [SMILES: CC(=O)Nc1cccc(-c2c(-c3cnn(C)c3)oc3ncnc(Oc4ccc(NC(=O)c5cccn(-c6ccc(F)cc6C)c5=O)cc4)c23)c1] | 670 |
| 75 | 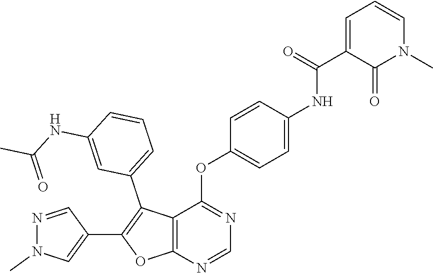 [SMILES: CC(=O)Nc1cccc(-c2c(-c3cnn(C)c3)oc3ncnc(Oc4ccc(NC(=O)c5cccn(C)c5=O)cc4)c23)c1] | 576 |
| 76 | 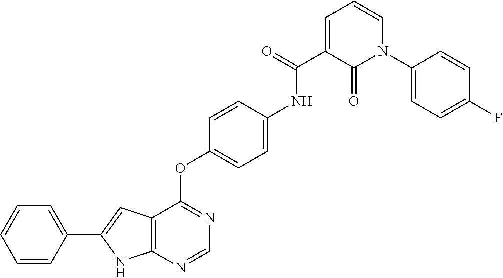 [SMILES: O=C(Nc1ccc(Oc2ncnc3[nH]c(-c4ccccc4)cc23)cc1)c1cccn(-c2ccc(F)cc2)c1=O] | 518 |
| 77 | 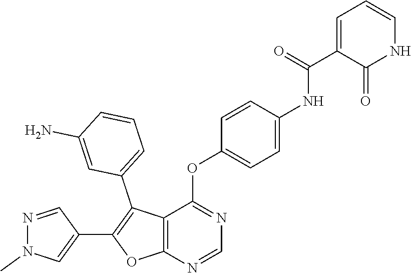 [SMILES: Cn1cc(-c2oc3ncnc(Oc4ccc(NC(=O)c5ccc[nH]c5=O)cc4)c3c2-c2cccc(N)c2)cn1] | 520 |
| 78 | 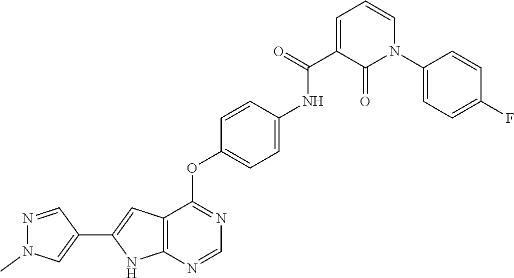 [SMILES: Cn1cc(-c2cc3c(Oc4ccc(NC(=O)c5cccn(-c6ccc(F)cc6)c5=O)cc4)ncnc3[nH]2)cn1] | 522 |
| 79 | 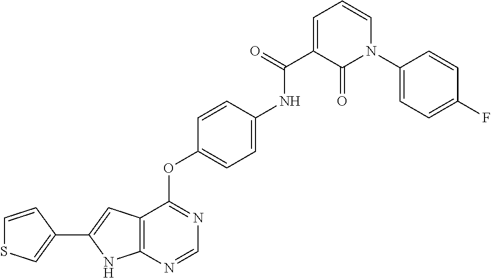 [SMILES: O=C(Nc1ccc(Oc2ncnc3[nH]c(-c4ccsc4)cc23)cc1)c1cccn(-c2ccc(F)cc2)c1=O] | 524 |
| 80 | 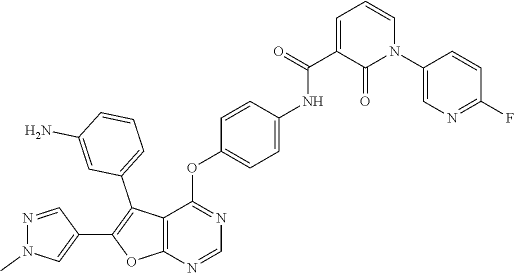 [SMILES: Cn1cc(-c2oc3ncnc(Oc4ccc(NC(=O)c5cccn(-c6ccc(F)nc6)c5=O)cc4)c3c2-c2cccc(N)c2)cn1] | 615 |
| 81 | 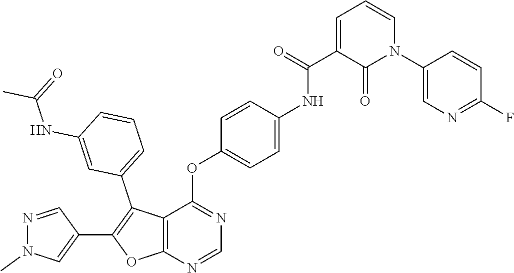 [SMILES: CC(=O)Nc1cccc(-c2c(-c3cnn(C)c3)oc3ncnc(Oc4ccc(NC(=O)c5cccn(-c6ccc(F)nc6)c5=O)cc4)c23)c1] | 657 |
| 82 | 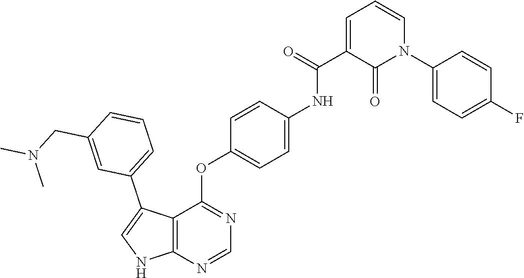 [SMILES: CN(C)Cc1cccc(-c2c[nH]c3ncnc(Oc4ccc(NC(=O)c5cccn(-c6ccc(F)cc6)c5=O)cc4)c23)c1] | 575 |
| 83 | 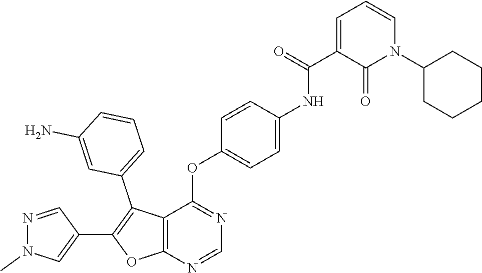 [SMILES: Cn1cc(-c2oc3ncnc(Oc4ccc(NC(=O)c5cccn(C6CCCCC6)c5=O)cc4)c3c2-c2cccc(N)c2)cn1] | 602 |
| 84 | 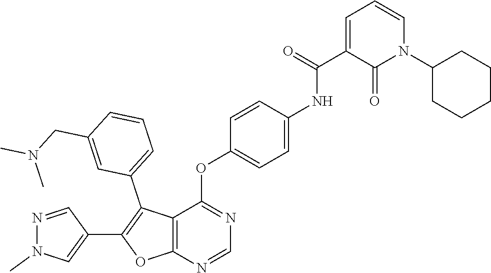 [SMILES: CN(C)Cc1cccc(-c2c(-c3cnn(C)c3)oc3ncnc(Oc4ccc(NC(=O)c5cccn(C6CCCCC6)c5=O)cc4)c23)c1] | 644 |
| 85 | 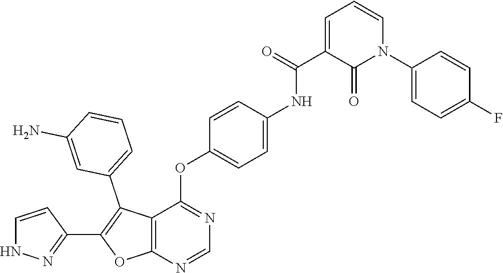 [SMILES: Nc1cccc(-c2c(-c3cc[nH]n3)oc3ncnc(Oc4ccc(NC(=O)c5cccn(-c6ccc(F)cc6)c5=O)cc4)c23)c1] | 600 |
| 86 | 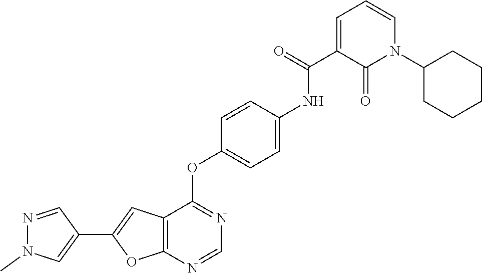 [SMILES: Cn1cc(-c2cc3c(Oc4ccc(NC(=O)c5cccn(C6CCCCC6)c5=O)cc4)ncnc3o2)cn1] | 511 |
| 87 | 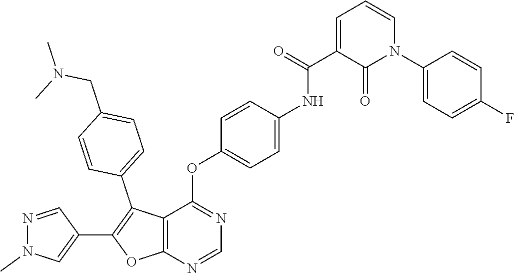 [SMILES: CN(C)Cc1ccc(-c2c(-c3cnn(C)c3)oc3ncnc(Oc4ccc(NC(=O)c5cccn(-c6ccc(F)cc6)c5=O)cc4)c23)cc1] | 656 |
| 88 | 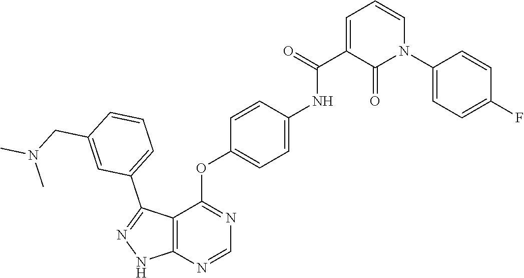 [SMILES: CN(C)Cc1cccc(-c2n[nH]c3ncnc(Oc4ccc(NC(=O)c5cccn(-c6ccc(F)cc6)c5=O)cc4)c23)c1] | 576 |
| 89 | 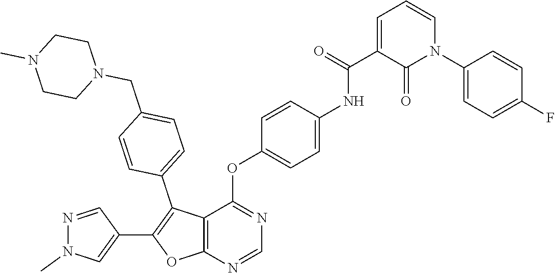 [SMILES: CN1CCN(Cc2ccc(-c3c(-c4cnn(C)c4)oc4ncnc(Oc5ccc(NC(=O)c6cccn(-c7ccc(F)cc7)c6=O)cc5)c34)cc2)CC1] | 711 |
| 90 | 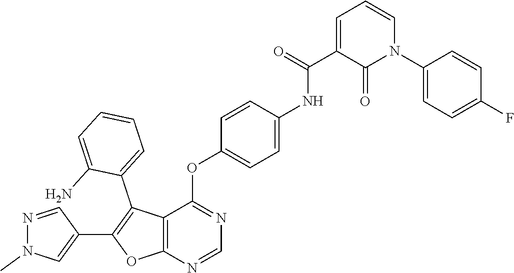 [SMILES: Cn1cc(-c2oc3ncnc(Oc4ccc(NC(=O)c5cccn(-c6ccc(F)cc6)c5=O)cc4)c3c2-c2ccccc2N)cn1] | 614 |
| 91 | 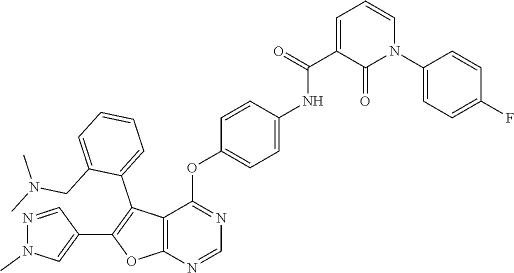 [SMILES: CN(C)Cc1ccccc1-c1c(-c2cnn(C)c2)oc2ncnc(Oc3ccc(NC(=O)c4cccn(-c5ccc(F)cc5)c4=O)cc3)c12] | 656 |
| 92 | 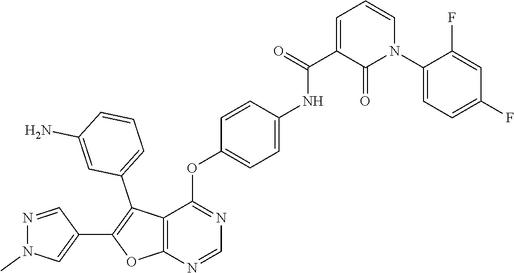 [SMILES: Cn1cc(-c2oc3ncnc(Oc4ccc(NC(=O)c5cccn(-c6ccc(F)cc6F)c5=O)cc4)c3c2-c2cccc(N)c2)cn1] | 632 |
| 93 | 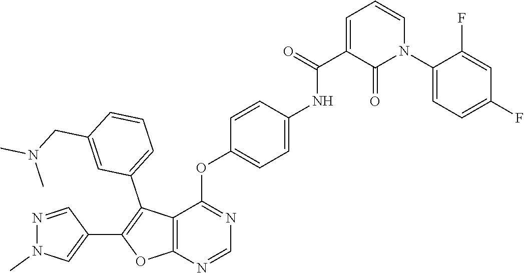 [SMILES: CN(C)Cc1cccc(-c2c(-c3cnn(C)c3)oc3ncnc(Oc4ccc(NC(=O)c5cccn(-c6ccc(F)cc6F)c5=O)cc4)c23)c1] | 674 |
| 94 | 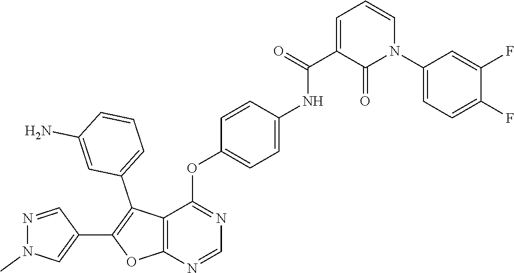 [SMILES: Cn1cc(-c2oc3ncnc(Oc4ccc(NC(=O)c5cccn(-c6ccc(F)c(F)c6)c5=O)cc4)c3c2-c2cccc(N)c2)cn1] | 632 |
| 95 | 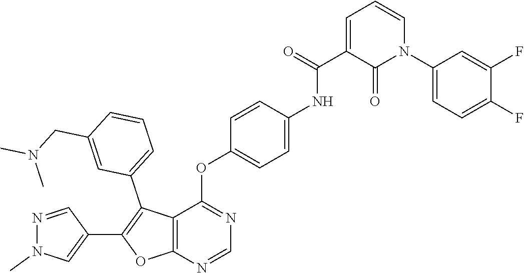 [SMILES: CN(C)Cc1cccc(-c2c(-c3cnn(C)c3)oc3ncnc(Oc4ccc(NC(=O)c5cccn(-c6ccc(F)c(F)c6)c5=O)cc4)c23)c1] | 674 |
| 96 | 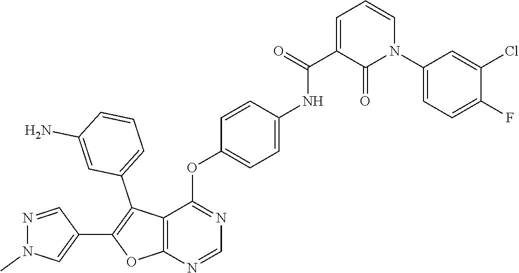 [SMILES: Cn1cc(-c2oc3ncnc(Oc4ccc(NC(=O)c5cccn(-c6ccc(F)c(Cl)c6)c5=O)cc4)c3c2-c2cccc(N)c2)cn1] | 649 |
| 97 | 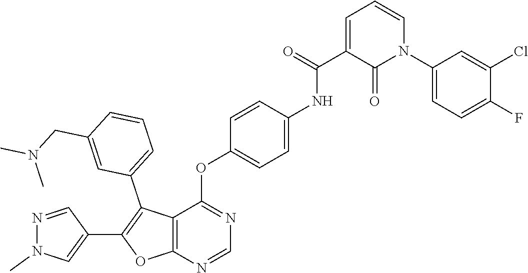 [SMILES: CN(C)Cc1cccc(-c2c(-c3cnn(C)c3)oc3ncnc(Oc4ccc(NC(=O)c5cccn(-c6ccc(F)c(Cl)c6)c5=O)cc4)c23)c1] | 691 |
| 98 | 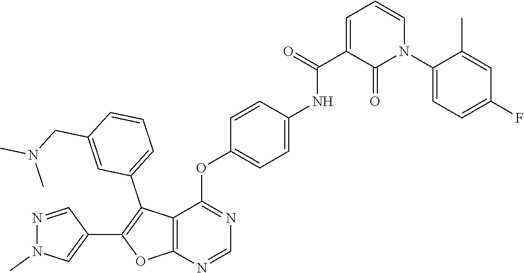 [SMILES: Cc1cc(F)ccc1-n1cccc(C(=O)Nc2ccc(Oc3ncnc4oc(-c5cnn(C)c5)c(-c5cccc(CN(C)C)c5)c34)cc2)c1=O] | 670 |
| 99 | 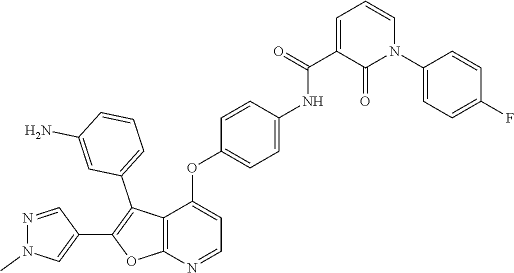 [SMILES: Cn1cc(-c2oc3nccc(Oc4ccc(NC(=O)c5cccn(-c6ccc(F)cc6)c5=O)cc4)c3c2-c2cccc(N)c2)cn1] | 613 |
| 100 | 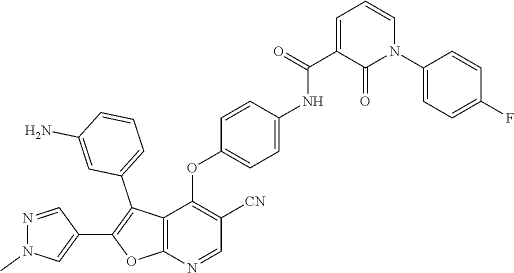 [SMILES: Cn1cc(-c2oc3ncc(C#N)c(Oc4ccc(NC(=O)c5cccn(-c6ccc(F)cc6)c5=O)cc4)c3c2-c2cccc(N)c2)cn1] | 638 |
| 101 | 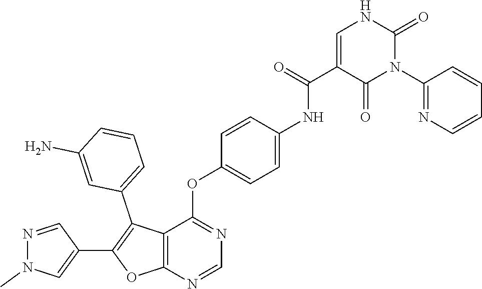 [SMILES: Cn1cc(-c2oc3ncnc(Oc4ccc(NC(=O)c5c[nH]c(=O)n(-c6ccccn6)c5=O)cc4)c3c2-c2cccc(N)c2)cn1] | 614 |
| 102 | 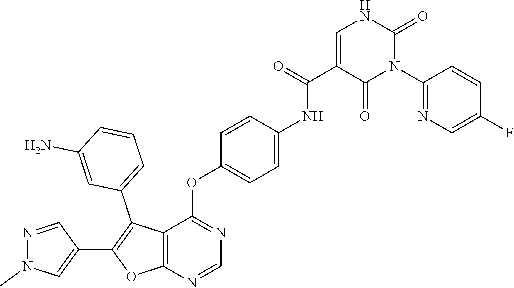 [SMILES: Cn1cc(-c2oc3ncnc(Oc4ccc(NC(=O)c5c[nH]c(=O)n(-c6ccc(F)cn6)c5=O)cc4)c3c2-c2cccc(N)c2)cn1] | 632 |
| 103 | 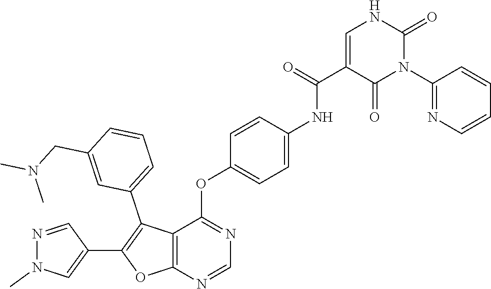 [SMILES: CN(C)Cc1cccc(-c2c(-c3cnn(C)c3)oc3ncnc(Oc4ccc(NC(=O)c5c[nH]c(=O)n(-c6ccccn6)c5=O)cc4)c23)c1] | 656 |
| 104 | 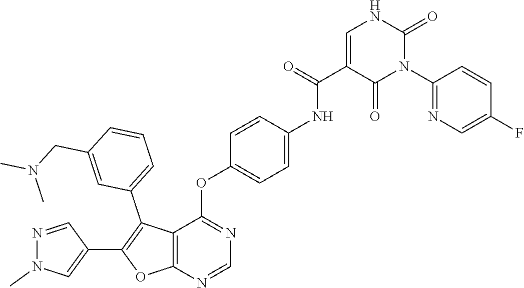 [SMILES: CN(C)Cc1cccc(-c2c(-c3cnn(C)c3)oc3ncnc(Oc4ccc(NC(=O)c5c[nH]c(=O)n(-c6ccc(F)cn6)c5=O)cc4)c23)c1] | 674 |
| 105 | 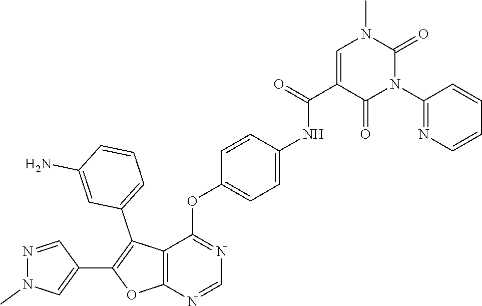 [SMILES: Cn1cc(-c2oc3ncnc(Oc4ccc(NC(=O)c5cn(C)c(=O)n(-c6ccccn6)c5=O)cc4)c3c2-c2cccc(N)c2)cn1] | 628 |
| 106 | 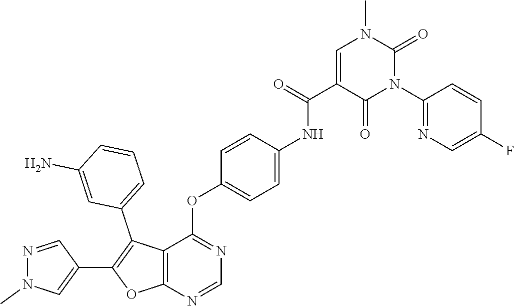 [SMILES: Cn1cc(-c2oc3ncnc(Oc4ccc(NC(=O)c5cn(C)c(=O)n(-c6ccc(F)cn6)c5=O)cc4)c3c2-c2cccc(N)c2)cn1] | 646 |
| 107 | 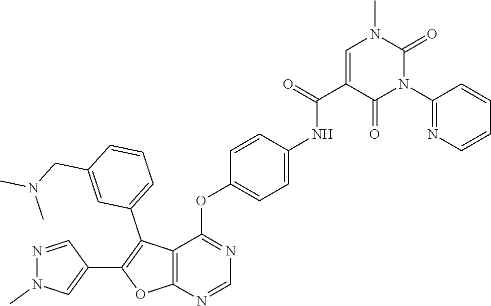 [SMILES: CN(C)Cc1cccc(-c2c(-c3cnn(C)c3)oc3ncnc(Oc4ccc(NC(=O)c5cn(C)c(=O)n(-c6ccccn6)c5=O)cc4)c23)c1] | 670 |
| 108 | 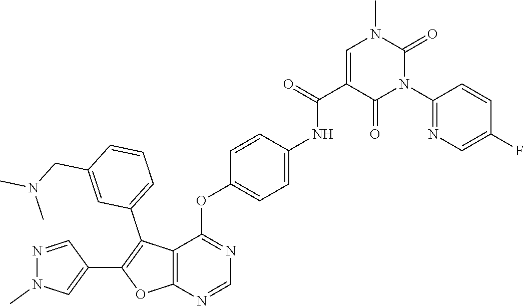 [SMILES: CN(C)Cc1cccc(-c2c(-c3cnn(C)c3)oc3ncnc(Oc4ccc(NC(=O)c5cn(C)c(=O)n(-c6ccc(F)cn6)c5=O)cc4)c23)c1] | 688 |
| 109 | 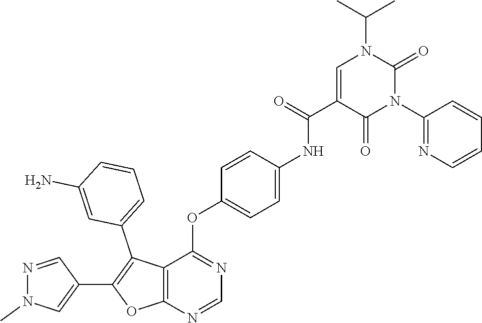 [SMILES: CC(C)n1cc(C(=O)Nc2ccc(Oc3ncnc4oc(-c5cnn(C)c5)c(-c5cccc(N)c5)c34)cc2)c(=O)n(-c2ccccn2)c1=O] | 656 |
| 110 | 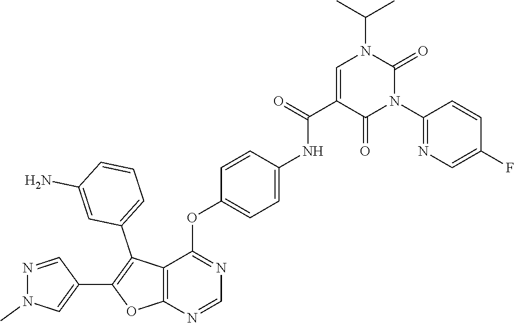 [SMILES: CC(C)n1cc(C(=O)Nc2ccc(Oc3ncnc4oc(-c5cnn(C)c5)c(-c5cccc(N)c5)c34)cc2)c(=O)n(-c2ccc(F)cn2)c1=O] | 674 |
| 111 | 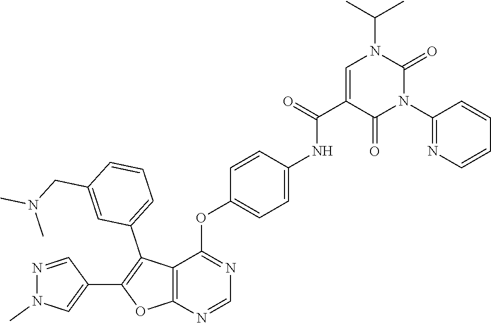 [SMILES: CC(C)n1cc(C(=O)Nc2ccc(Oc3ncnc4oc(-c5cnn(C)c5)c(-c5cccc(CN(C)C)c5)c34)cc2)c(=O)n(-c2ccccn2)c1=O] | 698 |
| 112 | 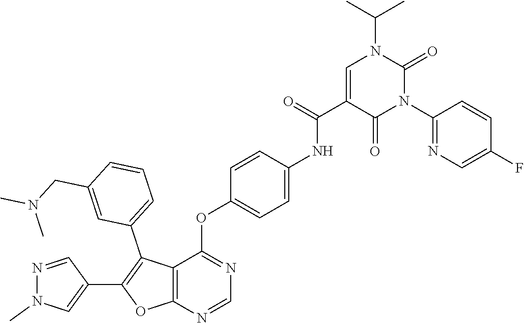 [SMILES: CC(C)n1cc(C(=O)Nc2ccc(Oc3ncnc4oc(-c5cnn(C)c5)c(-c5cccc(CN(C)C)c5)c34)cc2)c(=O)n(-c2ccc(F)cn2)c1=O] | 716 |
| 113 | 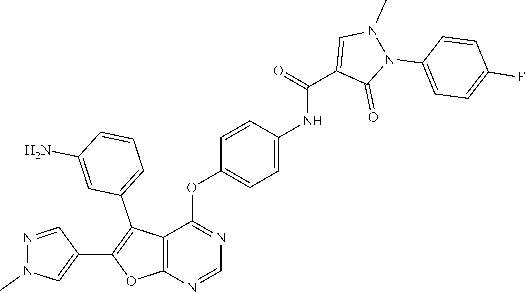 [SMILES: Cn1cc(-c2oc3ncnc(Oc4ccc(NC(=O)c5cn(C)n(-c6ccc(F)cc6)c5=O)cc4)c3c2-c2cccc(N)c2)cn1] | 617 |
| 114 | 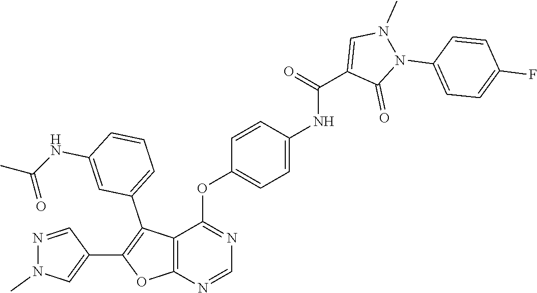 [SMILES: CC(=O)Nc1cccc(-c2c(-c3cnn(C)c3)oc3ncnc(Oc4ccc(NC(=O)c5cn(C)n(-c6ccc(F)cc6)c5=O)cc4)c23)c1] | 659 |
| 115 | 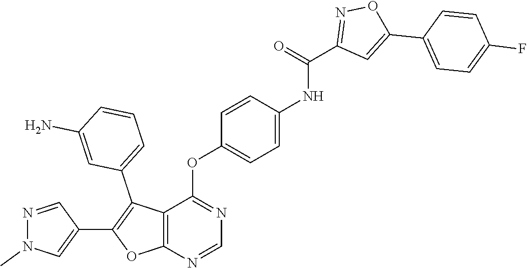 [SMILES: Cn1cc(-c2oc3ncnc(Oc4ccc(NC(=O)c5cc(-c6ccc(F)cc6)on5)cc4)c3c2-c2cccc(N)c2)cn1] | 588 |
| 116 | 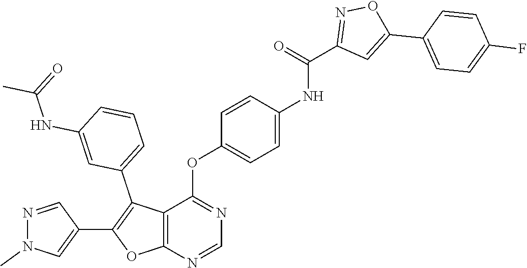 [SMILES: CC(=O)Nc1cccc(-c2c(-c3cnn(C)c3)oc3ncnc(Oc4ccc(NC(=O)c5cc(-c6ccc(F)cc6)on5)cc4)c23)c1] | 630 |
| 117 | 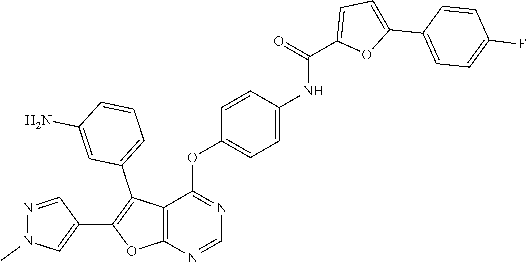 [SMILES: Cn1cc(-c2oc3ncnc(Oc4ccc(NC(=O)c5ccc(-c6ccc(F)cc6)o5)cc4)c3c2-c2cccc(N)c2)cn1] | 587 |
| 118 | 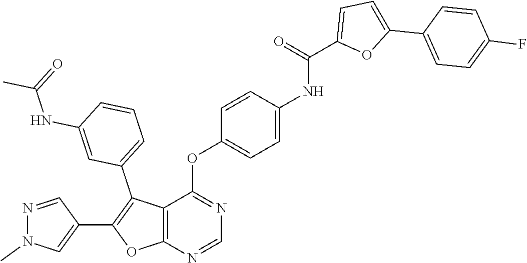 [SMILES: CC(=O)Nc1cccc(-c2c(-c3cnn(C)c3)oc3ncnc(Oc4ccc(NC(=O)c5ccc(-c6ccc(F)cc6)o5)cc4)c23)c1] | 629 |
| 119 | 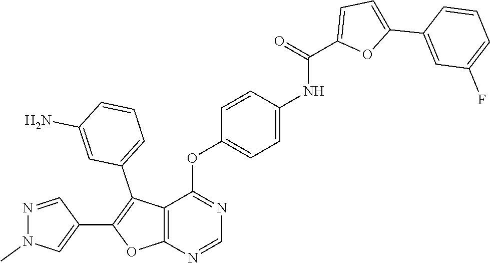 [SMILES: Cn1cc(-c2oc3ncnc(Oc4ccc(NC(=O)c5ccc(-c6cccc(F)c6)o5)cc4)c3c2-c2cccc(N)c2)cn1] | 587 |
| 120 | 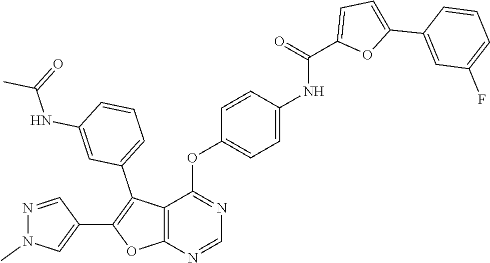 [SMILES: CC(=O)Nc1cccc(-c2c(-c3cnn(C)c3)oc3ncnc(Oc4ccc(NC(=O)c5ccc(-c6cccc(F)c6)o5)cc4)c23)c1] | 629 |
| 121 | 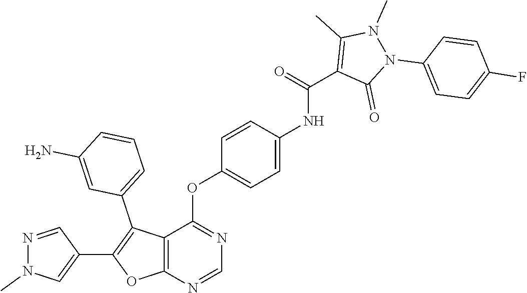 [SMILES: Cc1c(C(=O)Nc2ccc(Oc3ncnc4oc(-c5cnn(C)c5)c(-c5cccc(N)c5)c34)cc2)c(=O)n(-c2ccc(F)cc2)n1C] | 631 |
| 122 | 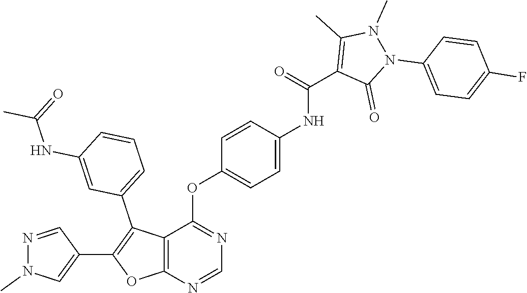 [SMILES: CC(=O)Nc1cccc(-c2c(-c3cnn(C)c3)oc3ncnc(Oc4ccc(NC(=O)c5c(C)n(C)n(-c6ccc(F)cc6)c5=O)cc4)c23)c1] | 673 |
| 123 | 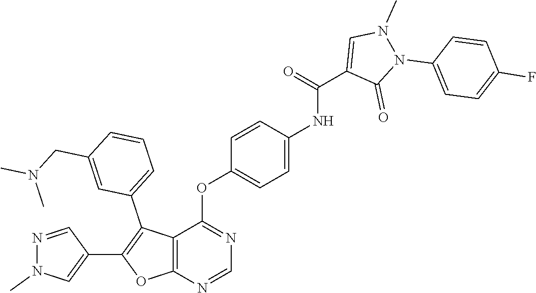 [SMILES: CN(C)Cc1cccc(-c2c(-c3cnn(C)c3)oc3ncnc(Oc4ccc(NC(=O)c5cn(C)n(-c6ccc(F)cc6)c5=O)cc4)c23)c1] | 659 |
| 124 | 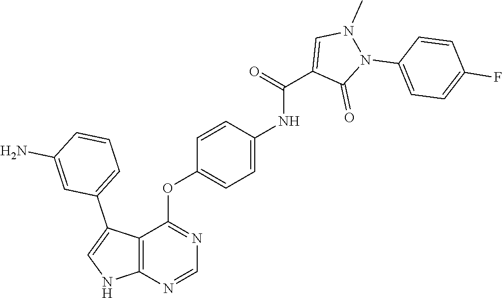 [SMILES: Cn1cc(C(=O)Nc2ccc(Oc3ncnc4[nH]cc(-c5cccc(N)c5)c34)cc2)c(=O)n1-c1ccc(F)cc1] | 536 |
| 125 | 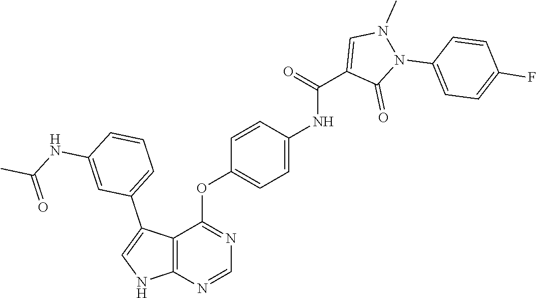 [SMILES: CC(=O)Nc1cccc(-c2c[nH]c3ncnc(Oc4ccc(NC(=O)c5cn(C)n(-c6ccc(F)cc6)c5=O)cc4)c23)c1] | 578 |
| 126 | 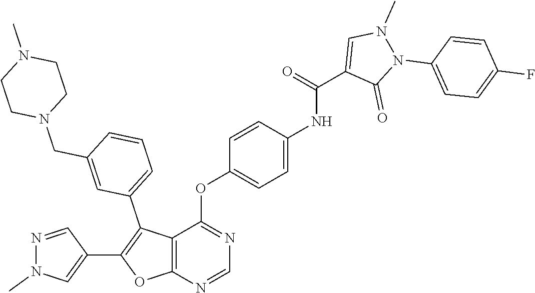 [SMILES: CN1CCN(Cc2cccc(-c3c(-c4cnn(C)c4)oc4ncnc(Oc5ccc(NC(=O)c6cn(C)n(-c7ccc(F)cc7)c6=O)cc5)c34)c2)CC1] | 714 |
| 127 | 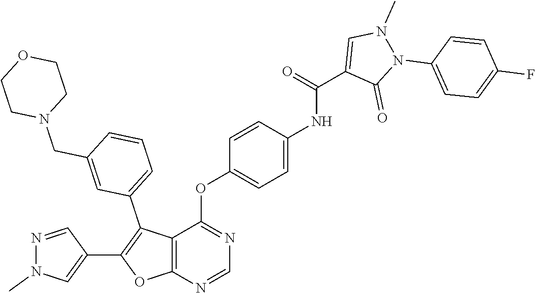 [SMILES: Cn1cc(-c2oc3ncnc(Oc4ccc(NC(=O)c5cn(C)n(-c6ccc(F)cc6)c5=O)cc4)c3c2-c2cccc(CN3CCOCC3)c2)cn1] | 701 |
| 128 | 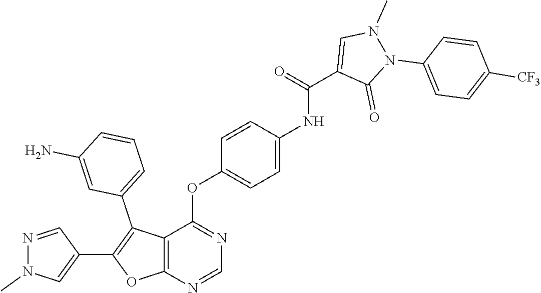 [SMILES: Cn1cc(-c2oc3ncnc(Oc4ccc(NC(=O)c5cn(C)n(-c6ccc(C(F)(F)F)cc6)c5=O)cc4)c3c2-c2cccc(N)c2)cn1] | 667 |
| 129 | 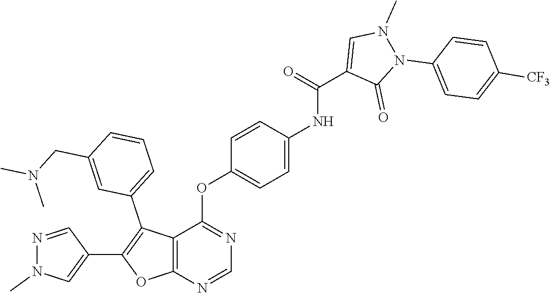 [SMILES: CN(C)Cc1cccc(-c2c(-c3cnn(C)c3)oc3ncnc(Oc4ccc(NC(=O)c5cn(C)n(-c6ccc(C(F)(F)F)cc6)c5=O)cc4)c23)c1] | 708 |
| 130 | 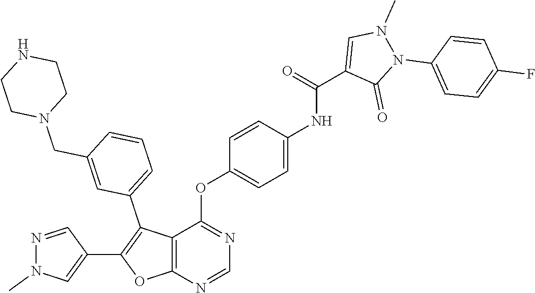 [SMILES: Cn1cc(-c2oc3ncnc(Oc4ccc(NC(=O)c5cn(C)n(-c6ccc(F)cc6)c5=O)cc4)c3c2-c2cccc(CN3CCNCC3)c2)cn1] | 700 |
| 131 | 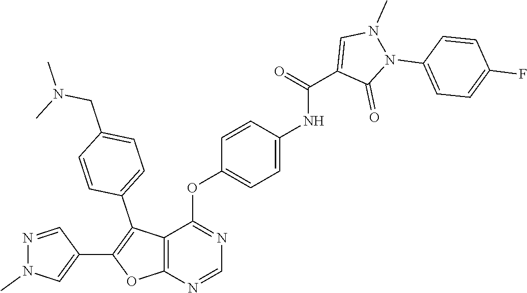 [SMILES: CN(C)Cc1ccc(-c2c(-c3cnn(C)c3)oc3ncnc(Oc4ccc(NC(=O)c5cn(C)n(-c6ccc(F)cc6)c5=O)cc4)c23)cc1] | 659 |
| 132 | 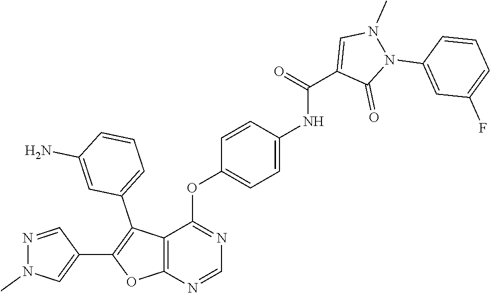 [SMILES: Cn1cc(-c2oc3ncnc(Oc4ccc(NC(=O)c5cn(C)n(-c6cccc(F)c6)c5=O)cc4)c3c2-c2cccc(N)c2)cn1] | 617 |
| 133 | 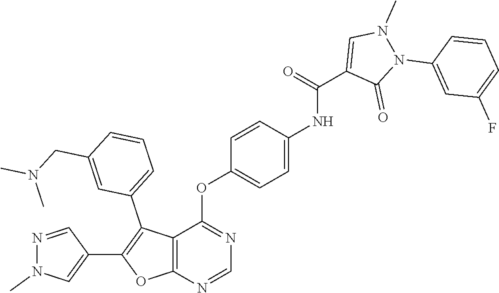 [SMILES: CN(C)Cc1cccc(-c2c(-c3cnn(C)c3)oc3ncnc(Oc4ccc(NC(=O)c5cn(C)n(-c6cccc(F)c6)c5=O)cc4)c23)c1] | 659 |
| 134 | 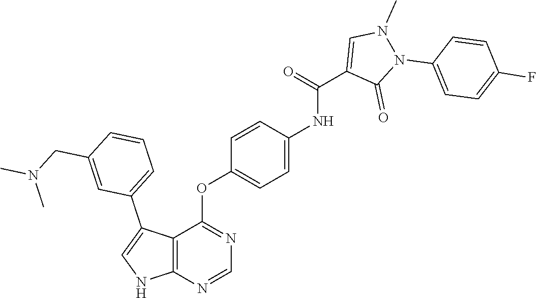 [SMILES: CN(C)Cc1cccc(-c2c[nH]c3ncnc(Oc4ccc(NC(=O)c5cn(C)n(-c6ccc(F)cc6)c5=O)cc4)c23)c1] | 578 |
| 135 | 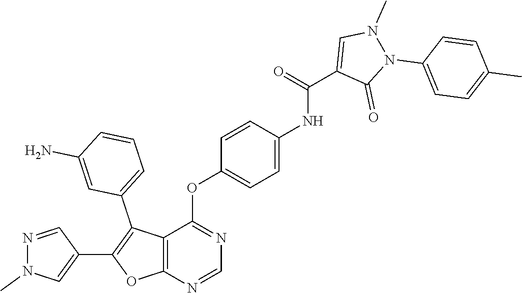 | 613 |
| 136 | 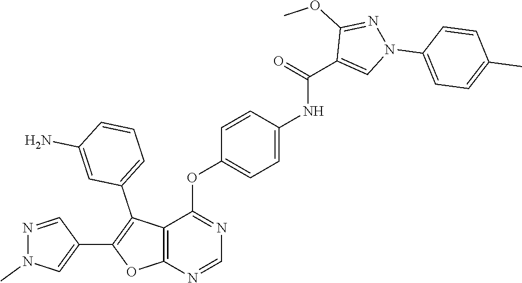 | 613 |
| 137 | 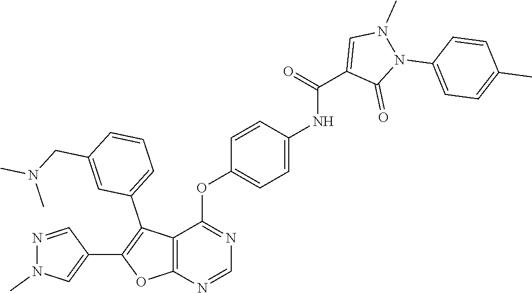 | 655 |
| 138 | 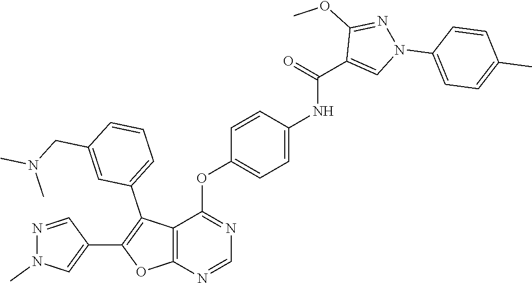 | 655 |
| 139 | 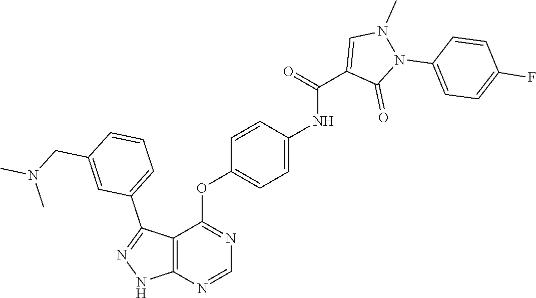 [SMILES: CN(C)Cc1cccc(-c2n[nH]c3ncnc(Oc4ccc(NC(=O)c5cn(C)n(-c6ccc(F)cc6)c5=O)cc4)c23)c1] | 579 |
| 140 | 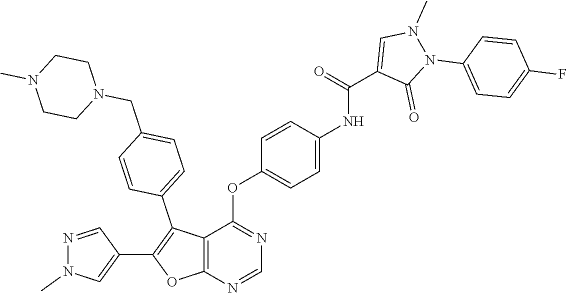 [SMILES: CN1CCN(Cc2ccc(-c3c(-c4cnn(C)c4)oc4ncnc(Oc5ccc(NC(=O)c6cn(C)n(-c7ccc(F)cc7)c6=O)cc5)c34)cc2)CC1] | 714 |
| 141 | 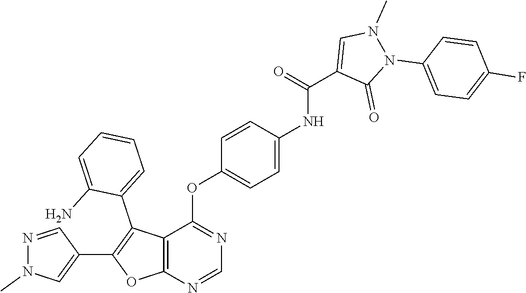 [SMILES: Cn1cc(-c2oc3ncnc(Oc4ccc(NC(=O)c5cn(C)n(-c6ccc(F)cc6)c5=O)cc4)c3c2-c2ccccc2N)cn1] | 617 |
| 142 | 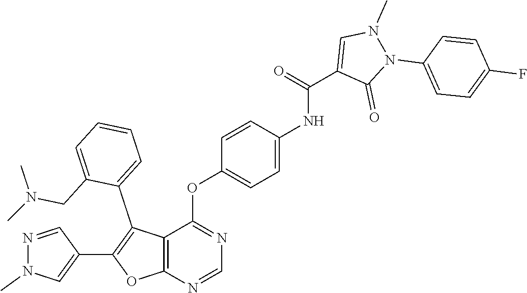 [SMILES: CN(C)Cc1ccccc1-c1c(-c2cnn(C)c2)oc2ncnc(Oc3ccc(NC(=O)c4cn(C)n(-c5ccc(F)cc5)c4=O)cc3)c12] | 659 |
| 143 | 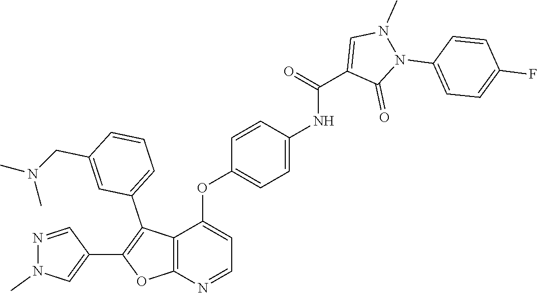 [SMILES: CN(C)Cc1cccc(-c2c(-c3cnn(C)c3)oc3nccc(Oc4ccc(NC(=O)c5cn(C)n(-c6ccc(F)cc6)c5=O)cc4)c23)c1] | 658 |
| 144 | 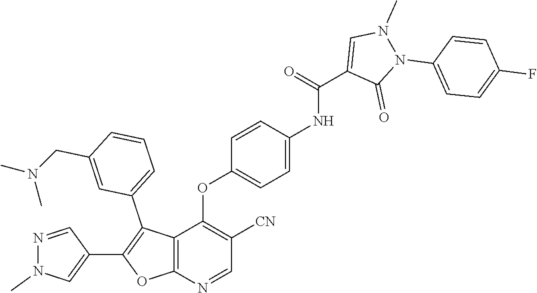 [SMILES: CN(C)Cc1cccc(-c2c(-c3cnn(C)c3)oc3ncc(C#N)c(Oc4ccc(NC(=O)c5cn(C)n(-c6ccc(F)cc6)c5=O)cc4)c23)c1] | 683 |
| 145 | 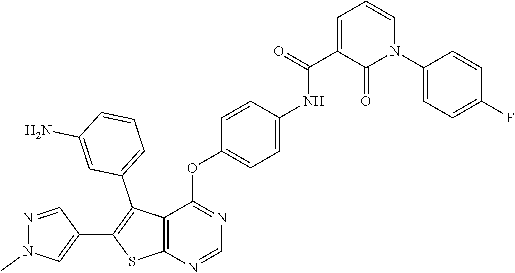 [SMILES: Cn1cc(-c2sc3ncnc(Oc4ccc(NC(=O)c5cccn(-c6ccc(F)cc6)c5=O)cc4)c3c2-c2cccc(N)c2)cn1] | 630 |
| 146 | 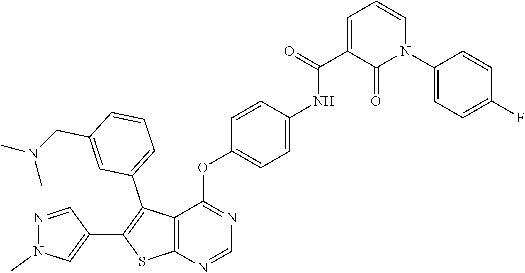 [SMILES: CN(C)Cc1cccc(-c2c(-c3cnn(C)c3)sc3ncnc(Oc4ccc(NC(=O)c5cccn(-c6ccc(F)cc6)c5=O)cc4)c23)c1] | 672 |
| 147 | 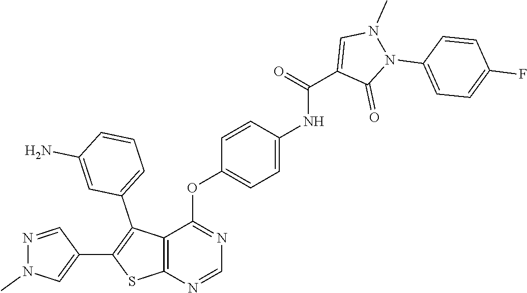 [SMILES: Cn1cc(-c2sc3ncnc(Oc4ccc(NC(=O)c5cn(C)n(-c6ccc(F)cc6)c5=O)cc4)c3c2-c2cccc(N)c2)cn1] | 632 |
| 148 | 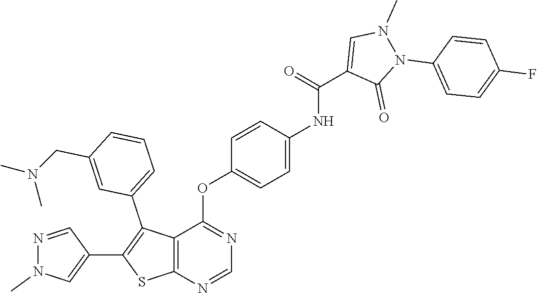 [SMILES: CN(C)Cc1cccc(-c2c(-c3cnn(C)c3)sc3ncnc(Oc4ccc(NC(=O)c5cn(C)n(-c6ccc(F)cc6)c5=O)cc4)c23)c1] | 674 |
| 149 | 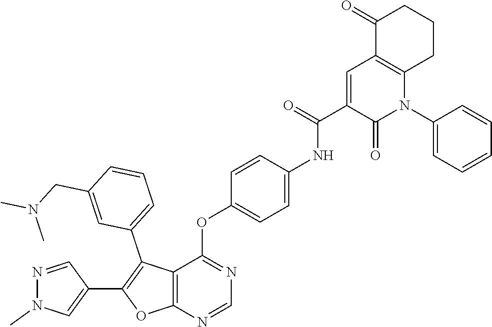 [SMILES: CN(C)Cc1cccc(-c2c(-c3cnn(C)c3)oc3ncnc(Oc4ccc(NC(=O)c5cc6c(n(-c7ccccc7)c5=O)CCCC6=O)cc4)c23)c1] | 706 |
| 150 | 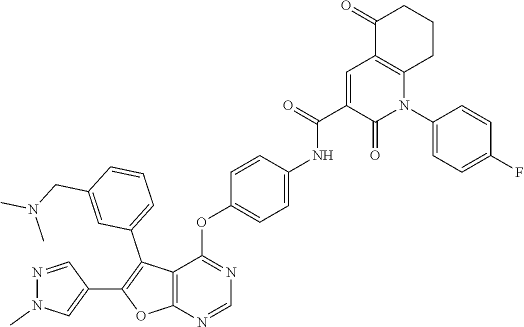 [SMILES: CN(C)Cc1cccc(-c2c(-c3cnn(C)c3)oc3ncnc(Oc4ccc(NC(=O)c5cc6c(n(-c7ccc(F)cc7)c5=O)CCCC6=O)cc4)c23)c1] | 724 |
| 151 | 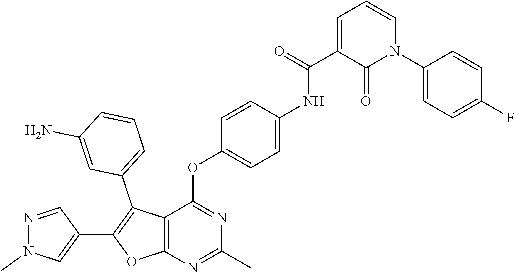 [SMILES: Cc1nc(Oc2ccc(NC(=O)c3cccn(-c4ccc(F)cc4)c3=O)cc2)c2c(-c3cccc(N)c3)c(-c3cnn(C)c3)oc2n1] | 628 |
| 152 | 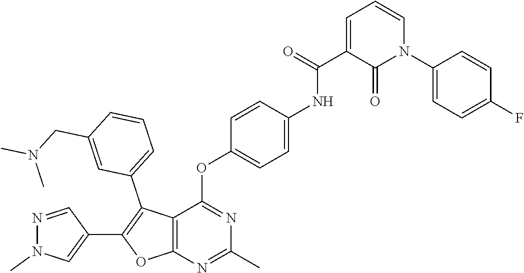 [SMILES: Cc1nc(Oc2ccc(NC(=O)c3cccn(-c4ccc(F)cc4)c3=O)cc2)c2c(-c3cccc(CN(C)C)c3)c(-c3cnn(C)c3)oc2n1] | 670 |
| 153 | 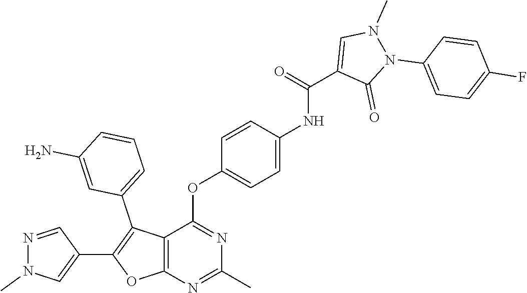 [SMILES: Cc1nc(Oc2ccc(NC(=O)c3cn(C)n(-c4ccc(F)cc4)c3=O)cc2)c2c(-c3cccc(N)c3)c(-c3cnn(C)c3)oc2n1] | 631 |
| 154 | 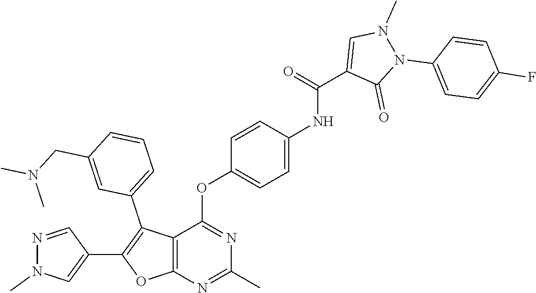 [SMILES: Cc1nc(Oc2ccc(NC(=O)c3cn(C)n(-c4ccc(F)cc4)c3=O)cc2)c2c(-c3cccc(CN(C)C)c3)c(-c3cnn(C)c3)oc2n1] | 673 |
| 155 | 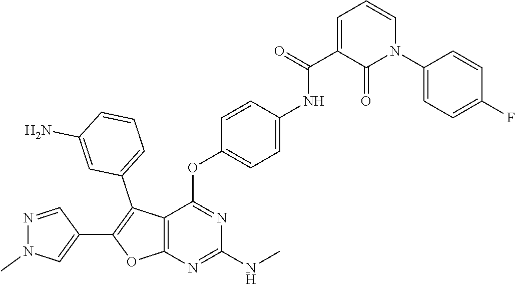 [SMILES: CNc1nc(Oc2ccc(NC(=O)c3cccn(-c4ccc(F)cc4)c3=O)cc2)c2c(-c3cccc(N)c3)c(-c3cnn(C)c3)oc2n1] | 643 |
| 156 | 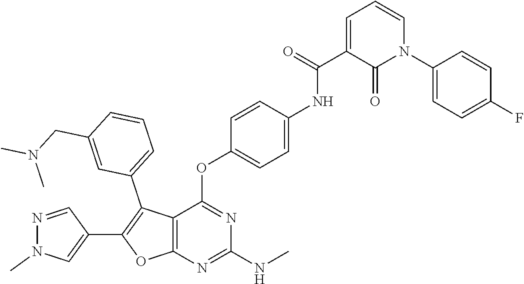 [SMILES: CNc1nc(Oc2ccc(NC(=O)c3cccn(-c4ccc(F)cc4)c3=O)cc2)c2c(-c3cccc(CN(C)C)c3)c(-c3cnn(C)c3)oc2n1] | 685 |
| 157 | 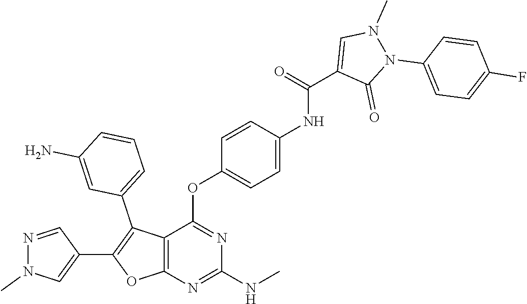 [SMILES: CNc1nc(Oc2ccc(NC(=O)c3cn(C)n(-c4ccc(F)cc4)c3=O)cc2)c2c(-c3cccc(N)c3)c(-c3cnn(C)c3)oc2n1] | 646 |
| 158 | 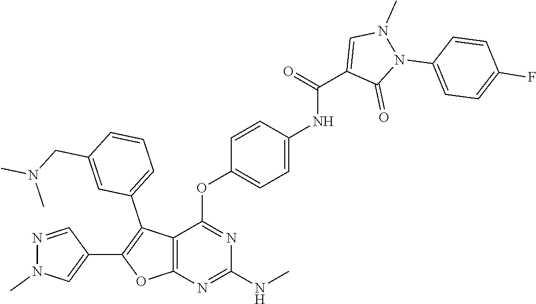 [SMILES: CNc1nc(Oc2ccc(NC(=O)c3cn(C)n(-c4ccc(F)cc4)c3=O)cc2)c2c(-c3cccc(CN(C)C)c3)c(-c3cnn(C)c3)oc2n1] | 688 |
| 159 | 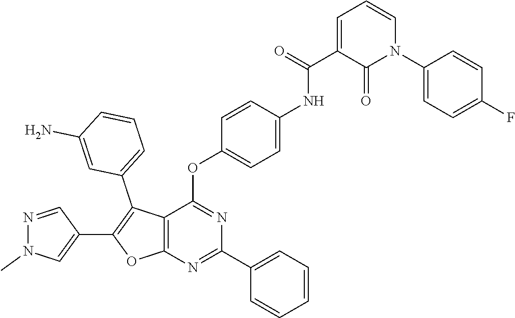 [SMILES: Cn1cc(-c2oc3nc(-c4ccccc4)nc(Oc4ccc(NC(=O)c5cccn(-c6ccc(F)cc6)c5=O)cc4)c3c2-c2cccc(N)c2)cn1] | 690 |
| 160 | 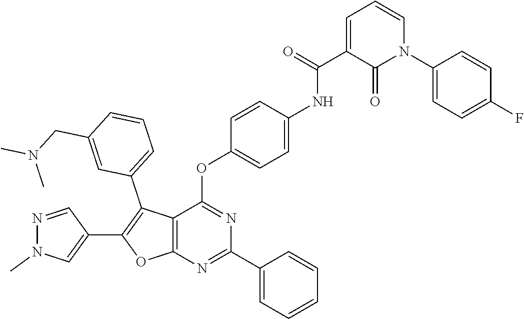 [SMILES: CN(C)Cc1cccc(-c2c(-c3cnn(C)c3)oc3nc(-c4ccccc4)nc(Oc4ccc(NC(=O)c5cccn(-c6ccc(F)cc6)c5=O)cc4)c23)c1] | 732 |
| 161 | 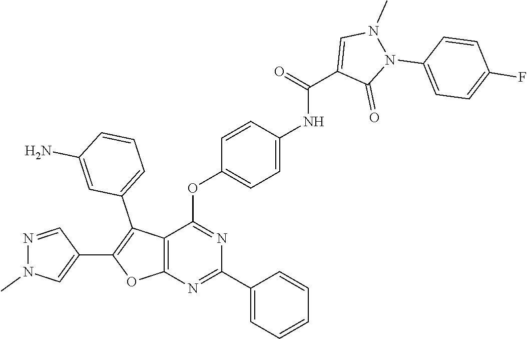 [SMILES: Cn1cc(-c2oc3nc(-c4ccccc4)nc(Oc4ccc(NC(=O)c5cn(C)n(-c6ccc(F)cc6)c5=O)cc4)c3c2-c2cccc(N)c2)cn1] | 693 |
| 162 | 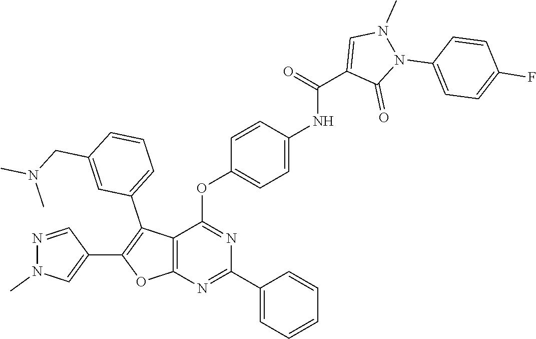 [SMILES: CN(C)Cc1cccc(-c2c(-c3cnn(C)c3)oc3nc(-c4ccccc4)nc(Oc4ccc(NC(=O)c5cn(C)n(-c6ccc(F)cc6)c5=O)cc4)c23)c1] | 735 |
| 163 | 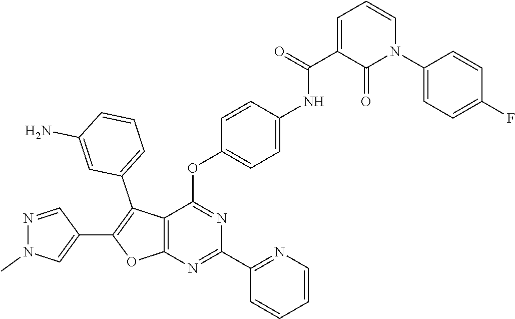 [SMILES: Cn1cc(-c2oc3nc(-c4ccccn4)nc(Oc4ccc(NC(=O)c5cccn(-c6ccc(F)cc6)c5=O)cc4)c3c2-c2cccc(N)c2)cn1] | 691 |
| 164 | 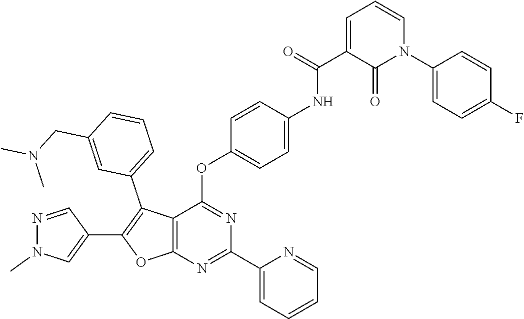 [SMILES: CN(C)Cc1cccc(-c2c(-c3cnn(C)c3)oc3nc(-c4ccccn4)nc(Oc4ccc(NC(=O)c5cccn(-c6ccc(F)cc6)c5=O)cc4)c23)c1] | 733 |
| 165 | 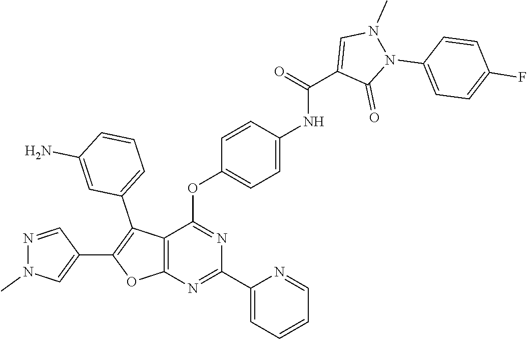 [SMILES: Cn1cc(-c2oc3nc(-c4ccccn4)nc(Oc4ccc(NC(=O)c5cn(C)n(-c6ccc(F)cc6)c5=O)cc4)c3c2-c2cccc(N)c2)cn1] | 694 |
| 166 | 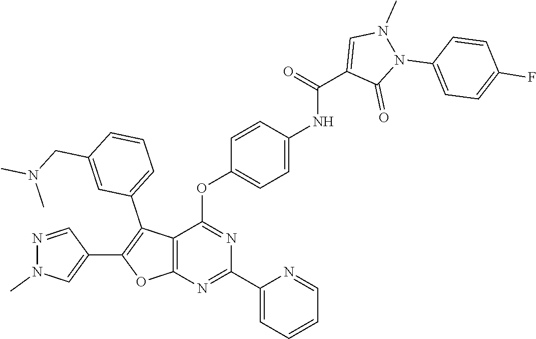 [SMILES: CN(C)Cc1cccc(-c2c(-c3cnn(C)c3)oc3nc(-c4ccccn4)nc(Oc4ccc(NC(=O)c5cn(C)n(-c6ccc(F)cc6)c5=O)cc4)c23)c1] | 736 |
| 167 | 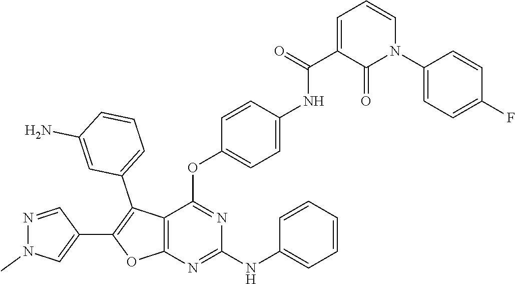 [SMILES: Cn1cc(-c2oc3nc(Nc4ccccc4)nc(Oc4ccc(NC(=O)c5cccn(-c6ccc(F)cc6)c5=O)cc4)c3c2-c2cccc(N)c2)cn1] | 705 |
| 168 | 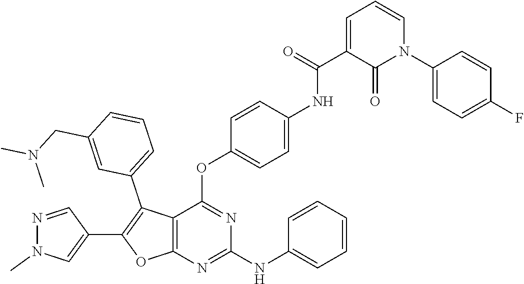 [SMILES: CN(C)Cc1cccc(-c2c(-c3cnn(C)c3)oc3nc(Nc4ccccc4)nc(Oc4ccc(NC(=O)c5cccn(-c6ccc(F)cc6)c5=O)cc4)c23)c1] | 747 |
| 169 | 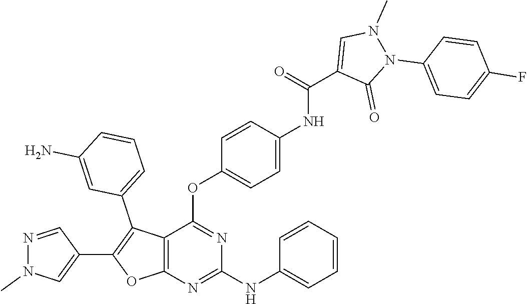 [SMILES: Cn1cc(-c2oc3nc(Nc4ccccc4)nc(Oc4ccc(NC(=O)c5cn(C)n(-c6ccc(F)cc6)c5=O)cc4)c3c2-c2cccc(N)c2)cn1] | 708 |
| 170 | 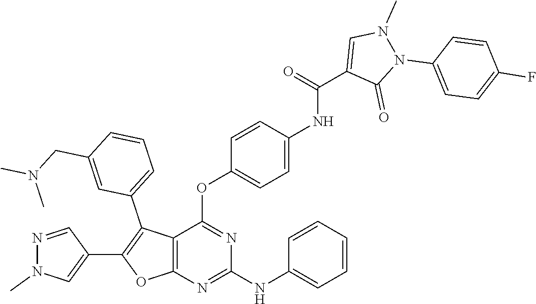 [SMILES: CN(C)Cc1cccc(-c2c(-c3cnn(C)c3)oc3nc(Nc4ccccc4)nc(Oc4ccc(NC(=O)c5cn(C)n(-c6ccc(F)cc6)c5=O)cc4)c23)c1] | 750 |
| 171 | 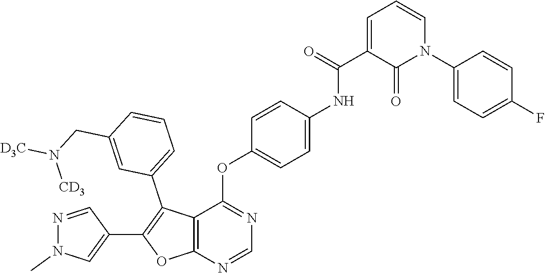 [SMILES: [2H]C([2H])([2H])N(Cc1cccc(-c2c(-c3cnn(C)c3)oc3ncnc(Oc4ccc(NC(=O)c5cccn(-c6ccc(F)cc6)c5=O)cc4)c23)c1)C([2H])([2H])[2H]] | 662 |
| 172 | 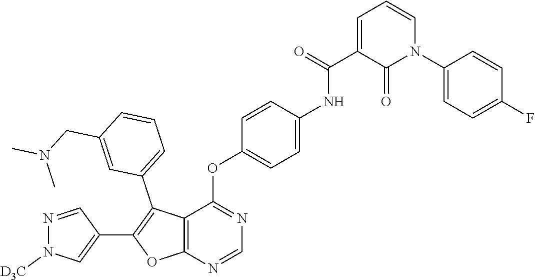 [SMILES: [2H]C([2H])([2H])n1cc(-c2oc3ncnc(Oc4ccc(NC(=O)c5cccn(-c6ccc(F)cc6)c5=O)cc4)c3c2-c2cccc(CN(C)C)c2)cn1] | 659 |
| 173 | 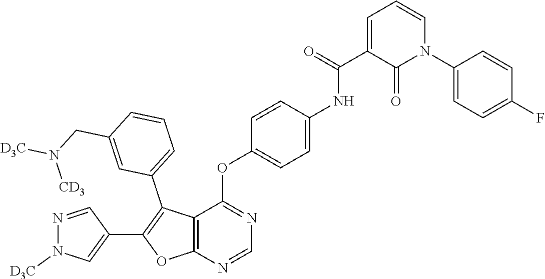 [SMILES: [2H]C([2H])([2H])N(Cc1cccc(-c2c(-c3cnn(C([2H])([2H])[2H])c3)oc3ncnc(Oc4ccc(NC(=O)c5cccn(-c6ccc(F)cc6)c5=O)cc4)c23)c1)C([2H])([2H])[2H]] | 665 |
| 174 | 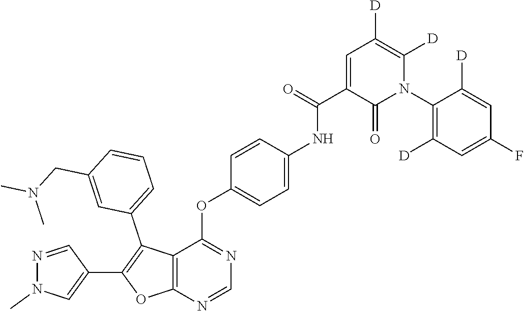 [SMILES: [2H]c1cc(F)cc([2H])c1-n1c([2H])c([2H])cc(C(=O)Nc2ccc(Oc3ncnc4oc(-c5cnn(C)c5)c(-c5cccc(CN(C)C)c5)c34)cc2)c1=O] | 660 |
| 175 | 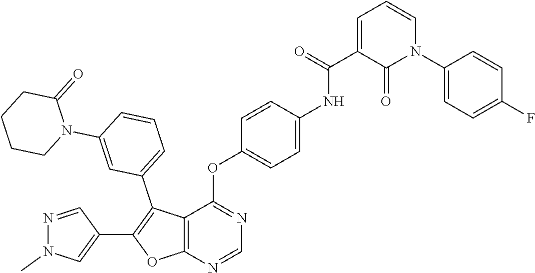 [SMILES: Cn1cc(-c2oc3ncnc(Oc4ccc(NC(=O)c5cccn(-c6ccc(F)cc6)c5=O)cc4)c3c2-c2cccc(N3CCCCC3=O)c2)cn1] | 696 |
| 176 | 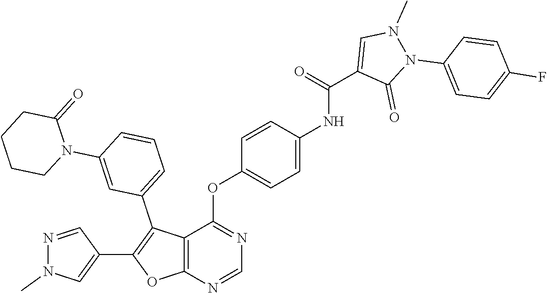 [SMILES: Cn1cc(-c2oc3ncnc(Oc4ccc(NC(=O)c5cn(C)n(-c6ccc(F)cc6)c5=O)cc4)c3c2-c2cccc(N3CCCCC3=O)c2)cn1] | 699 |
| 177 | 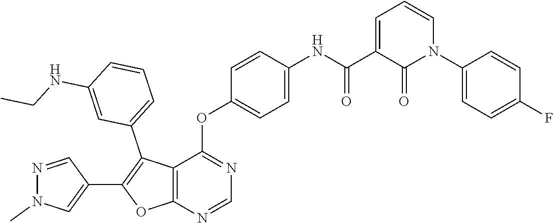 [SMILES: CCNc1cccc(-c2c(-c3cnn(C)c3)oc3ncnc(Oc4ccc(NC(=O)c5cccn(-c6ccc(F)cc6)c5=O)cc4)c23)c1] | 642 |
| 178 | 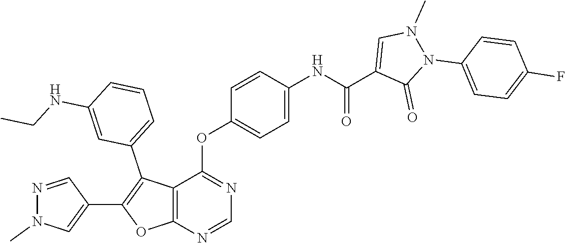 [SMILES: CCNc1cccc(-c2c(-c3cnn(C)c3)oc3ncnc(Oc4ccc(NC(=O)c5cn(C)n(-c6ccc(F)cc6)c5=O)cc4)c23)c1] | 645 |

Another subset of the compounds include N-(4-{[5-(3-aminophenyl)-7H-pyrrolo[2,3-d]pyrimidin-4-yl]oxy}phenyl)-1-(4-fluorophenyl)-2-oxo-1,2-dihydropyridine-3-carboxamide, N-(4-{[5-(3-aminophenyl)-6-(1-methyl-1H-pyrazol-4-yl)furo[2,3-d]pyrimidin-4-yl]oxy}phenyl)-1-(4-fluorophenyl)-2-oxo-1,2-dihydropyridine-3-carboxamide, N-(4-{[5-(4-aminophenyl)-6-(1-methyl-1H-pyrazol-4-yl)furo[2,3-d]pyrimidin-4-yl]oxy}phenyl)-1-(4-fluorophenyl)-2-oxo-1,2-dihydropyridine-3-carboxamide, N-{4-[(5-{3-[(dimethylamino)methyl]phenyl}-6-(1-methyl-1H-pyrazol-4-yl)furo[2,3-d]pyrimidin-4-yl)oxy]phenyl}-1-(4-fluorophenyl)-2-oxo-1,2-dihydro-pyridine-3-carboxamide, 1-(4-fluorophenyl)-N-[4-({5-[3-(methylamino)phenyl]-6-(1-methyl-1H-pyrazol-4-yl)furo[2,3-d]pyrimidin-4-yl}oxy)phenyl]-2-oxo-1,2-dihydropyridine-3-carboxamide, N-(4-{[5-(3-aminophenyl)-6-(1-methyl-1H-pyrazol-4-yl)furo[2,3-d]pyrimidin-4-yl]oxy}phenyl)-1-(4-fluorophenyl)-6-methyl-2-oxo-1,2-dihydropyridine-3-carboxamide, N-(4-{[5-(3-amino-phenyl)-6-(1-methyl-1H-pyrazol-4-yl)furo[2,3-d]pyrimidin-4-yl]oxy}phenyl)-1-(4-fluoro-phenyl)-4-methyl-2-oxo-1,2-dihydropyridine-3-carboxamide, N-{4-[(5-{3-[(dimethylamino)-methyl]phenyl}-7H-pyrrolo[2,3-d]pyrimidin-4-yl)oxy]phenyl}-1-(4-fluorophenyl)-2-oxo-1,2-dihydropyridine-3-carboxamide, 1-(3,4-difluorophenyl)-N-{4-[(5-{3-[(dimethylamino)methyl]-phenyl}-6-(1-methyl-1H-pyrazol-4-yl)furo[2,3-d]pyrimidin-4-yl)oxy]phenyl}-2-oxo-1,2-di-hydropyridine-3-carboxamide, N-(4-{[5-(3-aminophenyl)-6-(1-methyl-1H-pyrazol-4-yl)furo[2,3-d]pyrimidin-4-yl]oxy}phenyl)-2-(4-fluorophenyl)-1,5-dimethyl-3-oxo-2,3-dihydro-1H-pyrazole-4-carboxamide, and N-{4-[(5-{3-[(dimethylamino)methyl]phenyl}-6-(1-methyl-1H-pyrazol-4-yl)furo[2,3-d]pyrimidin-4-yl)oxy]phenyl}-2-(4-fluorophenyl)-1-methyl-3-oxo-2,3-dihydro-1H-pyrazole-4-carboxamide.

Also within the scope of this invention is a method of treating a TAM-associated disorder including the step of administering to a subject in need thereof an effective amount of any of the compounds described above.

Still within the scope of this invention is a pharmaceutical composition containing any of the compounds described above and a pharmaceutically acceptable carrier thereof.

The term “halo” herein refers to a fluoro, chloro, bromo, or iodo radical. A particular halogen is a fluoro radical (F). The term “amino” refers to a radical derived from amine, which is unsubstituted or mono-/di-substituted with alkyl, aryl, cycloalkyl, heterocycloalkyl, or heteroaryl. The term “aminoalkyl” refers to NH₂-alkyl, i.e., an alkyl that is substituted with at least one amino group. The term “alkylamino” refers to alkyl-NH—. Examples of aminoalkyl include aminomethyl and 2-aminoethyl. The term “acylamino” refers to —C(O)—NH—.

The term “alkyl” refers to a straight or branched hydrocarbon group, containing 1-20 carbon atoms (e.g., C₁₋₆) and a monovalent radical center derived by the removal of a hydrogen atom from a carbon atom of a parent alkane. Exemplary alkyl groups are methyl, ethyl, n-propyl, isopropyl, n-butyl, iso-butyl, tert-butyl, n-pentyl, and n-hexyl. The term “alkylcarbonyl” refers to alkyl-C(O)—. The term “haloalkyl” refers to alkyl substituted with one or more halo atoms. Examples include fluoromethyl, difluoromethyl, trifluoromethyl, fluoroethyl (e.g., 1-fluoroethyl and 2-fluoroethyl), difluoroethyl (e.g., 1,1-, 1,2-, and 2,2-difluoroethyl), and trifluoroethyl (e.g., 2,2,2-trifluoroethyl).

The term “alkoxy” refers to an —O-alkyl group. Examples are methoxy, ethoxy, propoxy, and isopropoxy. Alkoxy also includes haloalkoxy, namely, alkoxy substituted with one or more halogens, e.g., —O—CH₂Cl and —O—CHClCH₂Cl.

The term “alkylcarbonyl” refers to a —C(O)-alkyl group.

The term “cycloalkyl” refers to a nonaromatic, saturated or unsaturated monocyclic, bicyclic, tricyclic, or tetracyclic hydrocarbon group containing 3 to 12 carbons (e.g., C₃₋₆ and C₃₋₁₀). Examples are cyclopropyl, cyclobutyl, cyclopentyl, cyclopentenyl, cyclohexyl, cyclohexenyl, cycloheptyl, and cyclooctyl. The term “heterocycloalkyl” refers to a nonaromatic, saturated or unsaturated, 3-8 membered monocyclic, 8-12 membered bicyclic, or 11-14 membered tricyclic ring system having one or more heteroatoms (e.g., O, N, P, and S). Examples include aziridinyl, azetidinyl, pyrrolidinyl, dihydrofuranyl, tetrahydrofuranyl, tetrahydropyranyl, tetrahydrothiophenyl, tetrahydro-2-H-thiopyran-1,1-dioxidyl, piperazinyl, piperidinyl, morpholinyl, imidazolidinyl, azepanyl, dihydrothiadiazolyl, dioxanyl, and quinuclidinyl. Both “cycloalkyl” and “heterocycloalkyl” also includes fused, bridged, and spiro ring systems.

The term “alkenyl” refers to a straight or branched, monovalent, unsaturated aliphatic chain having 2 to 20 carbon atoms (e.g., C₂₋₄, C₂₋₆, and C₂₋₁₀) and one or more carbon-carbon double bonds. Examples are ethenyl (also known as vinyl), 1-methylethenyl, 1-methyl-1-propenyl, 1-butenyl, 1-hexenyl, 2-methyl-2-propenyl, 1-propenyl, 2-propenyl, 2-butenyl, and 2-pentenyl. The term “alkenylene” refers to a straight or branched, bivalent, unsaturated aliphatic chain having 2 to 20 carbon atoms (e.g., C₂₋₄, C₂₋₆, and C₂₋₁₀) and one or more carbon-carbon double bonds.

The term “alkynyl” refers to a straight or branched aliphatic chain having 2 to 20 carbon atoms (e.g., C₂₋₄, C₂₋₆, and C₂₋₁₀) and one or more carbon-carbon triple bonds. Examples are ethynyl, 2-propynyl, 2-butynyl, 3-methylbutnyl, and 1-pentynyl. The term “alkynylene” refers to a straight or branched, bivalent, unsaturated aliphatic chain having 2 to 20 carbon atoms (e.g., C₂₋₄, C₂₋₆, and C₂₋₁₀) and one or more carbon-carbon triple bonds.

The term “aryl” refers a 6-carbon monocyclic, 10-carbon bicyclic, 14-carbon tricyclic aromatic ring system wherein each ring can have one or more (e.g., 1 to 10, 1 to 5, and 1 to 3) substituents. Examples include phenyl, biphenyl, 1- or 2-naphthyl, 1,2-dihydronaphthyl, 1,2,3,4-tetrahydronaphthyl, indenyl, and indanyl. The term “aralkyl” refers to alkyl substituted with an aryl group.

The term “heteroaryl” refers to an aromatic 5-8 membered monocyclic, 8-12 membered bicyclic, or 11-14 membered tricyclic ring system having one or more heteroatoms (e.g., O, N, P, and S). Examples include pyridinyl, pyrimidinyl, quinolinyl, isoquinolinyl, indolyl, benzimidazolyl, benzoxazolyl, benzothiophenyl, benzofuranyl, pyrazolyl, triazolyl, oxazolyl, thiadiazolyl, tetrazolyl, oxazolyl, isoxazolyl, carbazolyl, furyl, imidazolyl, thienyl, thiazolyl, and benzothiazolyl.

Alkyl, alkoxyl, cycloalkyl, heterocycloalkyl, aryl, aralkyl, and heteroaryl mentioned herein include both substituted and unsubstituted moieties, unless specified otherwise. Examples of a substituent include hydroxyl (OH), halo (e.g., F and Cl), amino (NH₂), cyano (CN), nitro (NO₂), alkyl, alkenyl, alkynyl, alkoxy, cycloalkyl, acylamino, alkylamino, aminoalkyl, haloalkyl (e.g., trifluoromethyl), heterocycloalkyl, alkoxycarbonyl, amido, carboxy (COOH), alkanesulfonyl, alkylcarbonyl, alkenylcarbonyl, carbamido, carbamyl, carboxyl, thioureido, thiocyanato, sulfonamido, aryl, arylamino, aralkyl, and heteroaryl. All substitutes can be further substituted.

The term “compound”, when referring to a compound of this invention, also includes its salts, solvates, and prodrugs. The pharmaceutically acceptable salts include those listed in Handbook of Pharmaceutical Salts: Properties, Selection and Use, 2^{nd} Revised Edition, P. H. Stahl and C. G. Wermuth (Eds.), Wiley-VCH, New York, (2011). In addition to pharmaceutically acceptable salts, other salts are contemplated in the invention. They may serve as intermediates in the purification of compounds or in the preparation of other pharmaceutically acceptable salts, or are useful for identification, characterization or purification of compounds of the invention. A solvate refers to a complex formed between an active compound and a pharmaceutically acceptable solvent. Examples of a pharmaceutically acceptable solvent include water, ethanol, isopropanol, ethyl acetate, acetic acid, and ethanolamine. A prodrug refers to a compound that, after administration, is metabolized into a pharmaceutically active drug. Examples of a prodrug include esters and other pharmaceutically acceptable derivatives.

The compounds of the present invention may contain one or more non-aromatic double bonds or asymmetric centers. Each of them occurs as a racemate or a racemic mixture, a single R enantiomer, a single S enantiomer, an individual diastereomer, a diastereometric mixture, a cis-isomer, or a trans-isomer. Compounds of such isomeric forms are within the scope of this invention. They can be present as a mixture or can be isolated using chiral synthesis or chiral separation technologies.

The present invention also features use of one or more of the above-described compounds for treating disorders associated with TAM (e.g., AXL, MERTK, or both).

The term “TAM” refers to a family of receptor tyrosine kinases including TYRO3, AXL, and MERTK.

The term “AXL” refers to Axl receptor tyrosine kinase, an enzyme encoded by the AXL gene ad expressed in tumor cells and tumor vasculature, as well as normal tissues including bone marrow stroma and myeloid cells.

The term “MERTK” refers to Mer receptor tyrosine kinase, an enzyme encoded by the MERTK gene.

The term “treating” or “treatment” refers to administering one or more of the compounds to a subject with the purpose to confer a therapeutic effect, e.g., to slow, interrupt, arrest, control, or stop of the progression of an existing disorder and/or symptoms thereof, but does not necessarily indicate a total elimination of all symptoms. “An effective amount” refers to the amount of a compound that is required to confer the therapeutic effect. Effective doses will vary, as recognized by those skilled in the art, depending on the types of symptoms treated, route of administration, excipient usage, and the possibility of co-usage with other therapeutic treatment.

The disorders include cancer, kidney diseases, immune system diseases, and circulatory system disease. Cancer is selected from the group consisting hepatocellular cancer, bladder cancer, breast cancer, cervical cancer, colorectal cancer, endometrial cancer, anal cancer, Merkel cell carcinoma, gastric cancer, head and neck cancer, kidney cancer, liver cancer, lung cancer, melanoma, ovarian cancer, prostate cancer, esophageal cancer, gall bladder cancer, pancreatic cancer, thyroid cancer, skin cancer, leukemia, multiple myeloma, chronic lymphocytic lymphoma, adult T cell leukemia, B-cell lymphoma, acute myelogenous leukemia, Hodgkin's or non-Hodgkin's lymphoma, Waldenstrom's macroglobulinemia, hairy cell lymphoma, Burkett's lymphoma, glioblastoma, melanoma, and rhabdosarcoma. Preferably, the cancer is breast cancer, lung cancer, acute myelocytic leukemia, or colorectal cancer.

The term “subject” refers to an animal including human or non-human, such as a mammal. A human is a preferred subject.

A compound of this invention may be administered alone or in the form of a pharmaceutical composition with pharmaceutically acceptable carriers, diluents or excipients. Such pharmaceutical compositions and processes for making the same are known in the art (See, e.g., Remington: The Science and Practice of Pharmacy, A. Adejare, Editor, 23rd Edition., Academic Press, 2020).

To practice the method of the present invention, a composition or a kit containing one or more of the above-described compounds can be administered alone or co-administered with at least one other pharmacologically active substance simultaneously, concurrently, sequentially, successively, alternately, or separately. Simultaneous administration, also referring to as concomitant administration, includes administration at substantially the same time. Concurrent administration includes administering the active agents within the same general time period, for example on the same day(s) but not necessarily at the same time. Alternate administration includes administration of one agent during a time period, for example over the course of a few days or a week, followed by administration of the other agent(s) during a subsequent period of time, for example over the course of a few days or a week, and then repeating the pattern for one or more cycles. Sequential or successive administration includes administration of one agent during a first time period (for example over the course of a few days or a week) using one or more doses, followed by administration of the other agent(s) during a second and/or additional time period (for example over the course of a few days or a week) using one or more doses. An overlapping schedule may also be employed, which includes administration of the active agents on different days over the treatment period, not necessarily according to a regular sequence. Variations on these general guidelines may also be employed, e.g., according to the agents used and the condition of the subject.

The elements of the combinations of this invention may be administered (whether dependently or independently) by methods customary to the skilled person, e.g., by oral, enteral, parenteral, nasal, vaginal, rectal, or topical routes of administration and may be formulated, alone or together, in suitable dosage unit formulations containing conventional non-toxic pharmaceutically acceptable carriers, excipients and/or vehicles appropriate for each route of administration.

The term “parenteral” as used herein refers to subcutaneous, intracutaneous, intravenous, intraperitoneal, intramuscular, intraarticular, intraarterial, intrasynovial, intrasternal, intrathecal, intralesional, or intracranial injection, as well as any suitable infusion technique.

A composition for oral administration can be any orally acceptable dosage form including capsules, tablets, emulsions and aqueous suspensions, dispersions, and solutions. In the case of tablets, commonly used carriers include lactose and corn starch. Lubricating agents, such as magnesium stearate, are also typically added. For oral administration in a capsule form, useful diluents include lactose and dried corn starch. When aqueous suspensions or emulsions are administered orally, the active ingredient can be suspended or dissolved in an oily phase combined with emulsifying or suspending agents. If desired, certain sweetening, flavoring, or coloring agents can be added.

A nasal aerosol or inhalation composition can be prepared according to techniques well known in the art of pharmaceutical formulation. For example, such a composition can be prepared as a solution in saline, employing benzyl alcohol or other suitable preservatives, absorption promoters to enhance bioavailability, fluorocarbons, and/or other solubilizing or dispersing agents.

A composition having one or more of the above-described compounds can also be administered in the form of suppositories for rectal administration.

The carrier in the pharmaceutical composition must be “acceptable” in the sense that it is compatible with the active ingredient of the composition (and preferably, capable of stabilizing the active ingredient) and not deleterious to the subject to be treated. One or more solubilizing agents can be utilized as pharmaceutical excipients for delivery of an active compound. Examples include colloidal silicon oxide, magnesium stearate, cellulose, sodium lauryl sulfate, and D&C Yellow #10.

The details of one or more embodiments of the invention are set forth in the description below. Other features, objects, and advantages of the invention will be apparent from the description and from the claims.

### DETAILED DESCRIPTION

The present invention is based on a surprising discovery that the compounds of formula (I) reproduced below are effective in inhibiting AXL/MERTK activity and treating disorders associated therewith, including cancer. In vivo studies have demonstrated their efficacy in treating cancer.

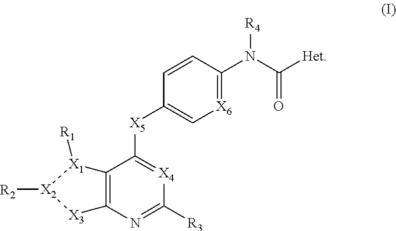

Variables R₁-R₄, X₁-X₆, and Het are defined above.

The compounds of formula (I) can be prepared by synthetic methods well known in the art. See, e.g., R. Larock, Comprehensive Organic Transformations (3^{rd} Ed., John Wiley and Sons 2018); P. G. M. Wuts and T. W. Greene, Greene's Protective Groups in Organic Synthesis (4^{th} Ed., John Wiley and Sons 2007); L. Fieser and M. Fieser, Fieser and Fieser's Reagents for Organic Synthesis (John Wiley and Sons 1994); and L. Paquette, ed., Encyclopedia of Reagents for Organic Synthesis (2^{nd} ed., John Wiley and Sons 2009) and subsequent editions thereof.

The compounds thus prepared can be purified following conventional methods such as crystallization, distillation/vacuum distillation, flash chromatography over silica, and preparative liquid chromatography.

Efficacy of the compounds of this invention can be initially determined using an in vitro method to identify their AXL and MERTK activity, all described in examples below. The selected compounds can be further tested to verify their in vivo efficacy, pharmacokinetic profiles, and toxicity, e.g., by administering it to an animal. Based on the results, an appropriate dosage range and administration route can be determined.

A compound of this invention is preferably formulated into a pharmaceutical composition containing a pharmaceutical carrier. The pharmaceutical composition is then given to a subject in need thereof to inhibit TAM (e.g., AXL and MERTK) thus treating disorders associated therewith, e.g., cancer.

Without further elaboration, it is believed that one skilled in the art can, based on the above description, utilize the present invention to its fullest extent. The following examples are to be construed as merely illustrative and not limitative of the remainder of the disclosure in any way whatsoever.

All publications cited herein are hereby incorporated by reference in their entirety.

Set forth below are examples illustrating preparation and efficacy evaluation of compounds of this invention.

### Examples 1-178: Syntheses of Compounds 1-178

Compounds 1-178 of this invention were prepared following the procedures provided below, with the synthesis of compound 11 as an example. The synthetic steps are illustrated in Scheme 1 below. Unless otherwise described, all reagents are commercially available from various suppliers such as MilliporeSigma (St. Louis, Missouri) and Fisher Chemical (Waltham, Massachusetts).

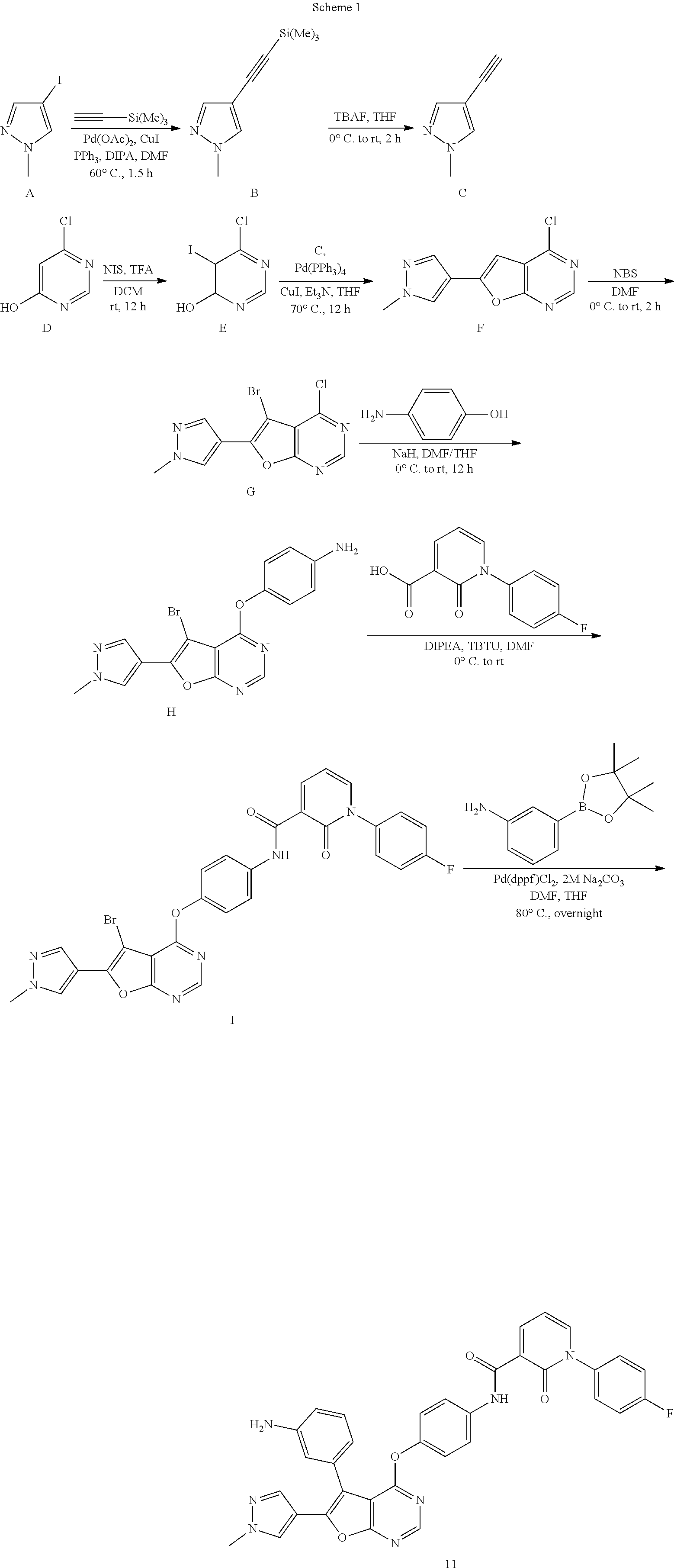

1-Methyl-4-[(trimethylsilyl)ethynyl]-1H-pyrazole (B). To a solution of 4-iodo-1-methyl-1H-pyrazole (5 g, 24.04 mmol) in dry N,N-dimethylformamide (DMF, 25 mL) was added trimethylsilylacetylene (5.13 mL, 36.06 mmol), diisopropylamine (DIPA, 4.4 mL, 31.25 mmol), CuI (275 mg, 1.44 mmol), triphenylphosphene (1.26 g, 4.81 mmol), and palladium(II) acetate (Pd(OAc)₂, 324 mg, 1.44 mmol). The reaction mixture was then heated at 60° C. and stirred for 1.5 hours. It was cooled to room temperature, diluted with water, and extracted with ether three times. The combined ether layers were dried over MgSO₄, filtered, and concentrated under reduced pressure. The crude thus obtained was purified by flash column chromatography (10-15% ethyl acetate in hexane) to yield compound B (2.9 g, 68%) as a brown liquid. ¹H NMR (400 MHz, CDCl₃) δ 7.57 (s, 1H), 7.48 (s, 1H), 3.86 (s, 3H), 0.20 (s, 9H). LRMS (ESI) m/z: 179.2 [M+H]⁺.

4-Ethynyl-1-methyl-1H-pyrazole (C). To a solution of compound B (2.9 g, 16.26 mmol) in tetrahydrofuran (THF, 40 mL) at 0° C. was added tetra-n-butylammonium fluoride (TBAF, 18 mL, 17.89 mmol). The reaction mixture was stirred at room temperature for 2 hours, concentrated under reduced pressure, diluted with water, and extracted three times with ether. The combined ether layers were dried over MgSO₄, filtered, and concentrated under reduced pressure. The crude thus obtained was purified by flash column chromatography (20% ethyl acetate in hexane) to yield compound C (1.3 g, 76%) as a yellow liquid.

¹H NMR (300 MHz, CDCl₃) δ 7.58 (s, 1H), 7.51 (s, 1H), 3.87 (s, 3H), 2.99 (s, 1H).

LRMS (ESI) m/z: 107.2 [M+H]⁺.

6-Chloro-5-iodo-4,5-dihydropyrimidin-4-ol (E). To a solution of 6-chloropyrimidin-4-ol (5 g, 38.31 mmol) in dichloromethane (DCM, 106 mL) was added N-iodosuccinimide (NIS, 9.48 g, 42.14 mmol) and trifluoroacetic acid (TFA, 21.3 mL, 69 mmol) dropwise at 0° C. The reaction mixture was stirred at room temperature for 12 hours and then concentrated under reduced pressure. The crude thus obtained was washed with water and ether followed by filtration to yield compound E (8.4 g, 85%) as a pink solid.

¹H NMR (400 MHz, DMSO-d₆) δ 13.18 (s, 1H), 8.16 (s, 1H).

LRMS (ESI) m/z: 256.1 [M+H]⁺.

4-Chloro-6-(1-methyl-1H-pyrazol-4-yl)furo[2,3-d]pyrimidine (F). To a solution of compound E (6.9 g, 26.91 mmol) in THE (90 mL) was added compound C (2.85 g, 26.91 mmol), Pd(PPh₃)₄ (1.55 g, 1.35 mmol), CuI (256 mg, 1.35 mmol) and triethylamine (11 mL, 80.7 mmol). The reaction mixture was stirred at 70° C. for 12 hours, cooled to room temperature, concentrated under reduced pressure, dissolved in ethyl acetate, and washed with water and brine. The organic layer was dried over MgSO₄, filtered, and concentrated under reduced pressure. The crude thus obtained was purified by flash column chromatography (15-20% ethyl acetate in hexane) to yield compound F (1.9 g, 30%) as a white solid.

¹H NMR (400 MHz, CDCl₃) δ 8.68 (s, 1H), 7.92 (s, 1H), 7.87 (s, 1H), 6.75 (s, 1H), 4.00 (s, 3H).

LRMS (ESI) m/z: 235.1 [M+H]⁺.

5-Bromo-4-chloro-6-(1-methyl-1H-pyrazol-4-yl)furo[2,3-d]pyrimidine (G). To a solution of compound F (1.9 g, 8.1 mmol) in DMF (20 mL) was added N-bromosuccinimide (NBS, 1.72 g, 9.72 mmol) at 0° C. The reaction mixture was stirred at 0° C. for 30 minutes, warmed to room temperature, and stirred for 2 hours. It was then diluted with water slowly and stirred for 30 minutes to precipitate a white solid, which was collected by filtration to yield compound G (1.85 g, 73%) as a white solid.

¹H NMR (400 MHz, CDCl₃) δ 8.70 (s, 1H), 8.22 (s, 1H), 8.18 (s, 1H), 4.03 (s, 3H).

LRMS (ESI) m/z: 313.0 [M+H]⁺.

4-{[5-Bromo-6-(1-methyl-1H-pyrazol-4-yl)furo[2,3-d]pyrimidin-4-yl]oxy}aniline (H). To a solution of sodium hydride (232 mg, 5.82 mmol) in DMF (10 mL) was added a solution of 4-aminophenol (577 mg, 5.29 mmol) in THE (10 mL) at 0° C. for 5 minutes, then a solution of compound G (1.65 g, 5.29 mmol) in THF (7.6 mL) and DMF (7.6 mL) at 0° C. The reaction mixture was stirred at room temperature for 12 hours before quenched with water (10 mL). The crude thus obtained was collected and stirred in water (50 mL) for 30 minutes to precipitate a solid, which was collected by filtration to yield compound H (1.85 g, 90.7%) as a brown solid.

¹H NMR (400 MHz, CDCl₃) δ 8.44 (s, 1H), 8.19 (s, 1H), 8.12 (s, 1H), 7.05 (d, J=8.8 Hz, 2H), 6.76 (d, J=8.8 Hz, 2H), 4.01 (s, 3H), 3.71 (s, 2H).

LRMS (ESI) m/z: 386.1 [M+H]⁺.

N-(4-{[5-bromo-6-(1-methyl-1H-pyrazol-4-yl)furo[2,3-d]pyrimidin-4-yl]oxy}phenyl)-1-(4-fluorophenyl)-2-oxo-1,2-dihydropyridine-3-carboxamide (I). To a solution of 1-(4-fluoro-phenyl)-2-oxo-1,2-dihydropyridine-3-carboxylic acid (1.45 g, 6.21 mmol) in DMF (84 mL) at 0° C. was added 2-(1H-benzotriazole-1-yl)-1,1,3,3-tetramethylaminium tetrafluoroborate (TBTU, 2.49 g, 7.77 mmol), N,N-diisopropylethylamine (DIPEA, 3.24 mL, 18.64 mmol), and compound H (2 g, 5.18 mmol). The reaction mixture was stirred at 0° C. for 2 hours, warmed to room temperature, stirred for 10 hours, diluted with to water (200 mL), and stirred for 20 minutes. The resulting precipitate was collected by filtration to yield compound I (3.1 g, 100%) as a white solid.

¹H NMR (400 MHz, CDCl₃) δ 11.91 (s, 1H), 8.76 (dd, J=7.2, 2.4 Hz, 1H), 8.45 (s, 1H), 8.20 (s, 1H), 8.13 (s, 1H), 7.85 (d, J=8.8 Hz, 2H), 7.61 (dd, J=6.6, 2.4 Hz, 1H), 7.45-7.37 (m, 2H), 7.31-7.21 (m, 3H), 6.61 (dd, J=7.2, 6.6 Hz, 1H), 4.02 (s, 3H).

LRMS (ESI) m/z: 601.1 [M+H]⁺.

N-(4-{[5-(3-aminophenyl)-6-(1-methyl-1H-pyrazol-4-yl)furo[2,3-d]pyrimidin-4-yl]oxy}phenyl)-1-(4-fluorophenyl)-2-oxo-1,2-dihydropyridine-3-carboxamide (11). To a solution of compound I (3.11 g, 5.17 mmol) in DMF (51.7 mL) and THE (51.7 mL) was added (3-aminophenyl)boronic acid (1.06 g, 7.76 mmol), Pd(dppf)Cl₂ (1.14 g, 1.55 mmol) and 2M Na₂CO_{3(aq)} (10.2 mL, 20.69 mmol). The resulting mixture was stirred at 110° C. for 16 hours under argon. Subsequently, it was cooled to room temperature, concentrated under reduced pressure, and purified by flash column chromatography (2-3% methanol in dichloromethane) to yield compound 11 (2.74 g, 86.4%) as a white solid.

¹H NMR (400 MHz, DMSO-d₆) δ 11.97 (s, 1H), 8.58 (dd, J=7.6, 2.4 Hz, 1H), 8.45 (s, 1H), 8.11 (dd, J=7.0, 2.4 Hz, 1H), 8.05 (d, J=0.8 Hz, 1H), 7.73 (d, J=9.0 Hz, 2H), 7.61 (dd, J=8.8, 4.8 Hz, 2H), 7.52 (d, J=0.8 Hz, 1H), 7.42 (dd, J=8.8, 8.4 Hz, 2H), 7.20 (d, J=9.0 Hz, 2H), 7.13 (dd, J=7.8, 7.6 Hz, 1H), 6.81 (dd, J=2.2, 1.6 Hz, 1H), 6.74 (ddd, J=7.6, 1.6, 1.0 Hz, 1H), 6.72 (dd, J=7.6, 7.0 Hz, 1H), 6.61 (ddd, J=7.8, 2.2, 1.0 Hz, 1H), 5.21 (s, 2H), 3.87 (s, 3H). LRMS (ESI) m/z: 614.3 [M+H]⁺.

HRMS (ESI) m/z: calcd for C₃₄H₂₄F₁N₇Na₁O₄, 636.1772 [M+Na]⁺, found 636.1771.

Compounds 1-10 and 12-178 were prepared following a similar procedure as described above with appropriate reagents either commercially available or prepared following procedures well-known in the art. Their ¹H-NMR data are provided below. See Table 2.

TABLE 2

| Compound | ¹H-NMR |
|---|---|
| 1 | (300 MHz, CDCl₃) δ 8.32 (s, 1H), 7.73-7.64 (m, 2H), 7.46 (d, J = 8.0 Hz, |
| | 1H), 7.38-7.06 (m, 11H), 6.79 (d, J = 7.5 Hz, 1H), 6.32 (d, J = 8.0 Hz, 1H), |
| | 4.30 (q, J = 7.2 Hz, 2H), 1.51 (t, J = 7.2 Hz, 3H). |
| 2 | (300 MHz, CD₃OD) δ 8.28 (s, 1H), 8.02-7.98 (m, 1H), 7.79-7.69 (m, 3H), |
| | 7.52-7.16 (m, 10H), 6.57 (d, J = 7.8 Hz, 1H), 4.31 (q, J = 6.9 Hz, 2H), 2.13 |
| | (s, 3H), 1.44 (t, J = 6.9 Hz, 3H). |
| 3 | (300 MHz, CD₃OD) δ 8.67 (dd, J = 7.0, 2.1 Hz, 1H), 8.25 (s, 1H), 7.94 (dd, |
| | J= 7.0, 2.1 Hz, 1H), 7.78-7.70 (m, 2H), 7.59-7.50 (m, 2H), 7.43 (s, 1H), |
| | 7.37-7.03 (m, 7H), 6.76-6.64 (m, 2H). |
| 4 | (300 MHz, CD₃OD) δ 8.67 (dd, J = 6.9, 2.1 Hz, 1H), 8.27 (s, 1H), 8.02-7.97 |
| | (m, 1H), 7.94 (dd, J = 6.9, 2.1 Hz, 1H), 7.78-7.70 (m, 2H), 7.58-7.26 (m, |
| | 8H), 7.25-7.17 (m, 2H), 6.72 (t, J = 6.9 Hz, 1H), 2.12 (s, 3H). |
| 7 | (300 MHz, DMSO-d₆) δ 11.95 (s, 1H), 10.06 (s, 1H), 8.58 (dd, J = 7.8, 1.8 |
| | Hz, 1H), 8.48 (s, 1H), 8.13-8.09 (m, 2H), 7.92 (s, 1H), 7.72 (d, J = 9.0 Hz, |
| | 2H), 7.63-7.56 (m, 4H), 7.45-7.31 (m, 4H), 7.19 (d, J = 9.0 Hz, 2H), 6.71 |
| | (t, J = 4.2 Hz, 1H), 3.87 (s, 3H). |
| 9 | (300 MHz, DMSO-d₆) δ 11.96 (s, 1H), 8.58 (d, J = 7.5 Hz, 1H), 8.52 (s, |
| | 1H), 8.10 (d, J = 4.8 Hz, 1H), 7.73 (d, J = 9.0 Hz, 2H), 7.61-7.60 (m, 4H), |
| | 7.44-7.41 (m, 5H), 7.20 (d, J = 9.0 Hz, 2H), 7.10 (t, J = 8.1 Hz, 1H), 6.76- |
| | 6.69 (m, 3H), 6.60 (d, J = 6.9 Hz, 1H), 5.18 (s, 2H). |
| 12 | (400 MHz, DMSO-d₆) δ 11.95 (s, 1H), 10.28 (s, 1H), 8.57 (dd, J = 7.2, 2.4 |
| | Hz, 1H), 8.48 (s, 1H), 8.14 (s, 1H), 8.10 (dd, J = 6.8, 2.0 Hz, 1H), 8.03 (s, |
| | 1H), 7.93-7.66 (m, 3H), 7.62-7.58 (m, 3H), 7.48-7.37 (m, 4H), 7.19 (d, J = |
| | 5.2 Hz, 2H), 6.73-6.69 (m, 1H), 6.44 (dd, J = 16.8, 10.0 Hz, 1H), 6.27 (dd, |
| | J = 16.8, 2.0 Hz, 1H), 5.76 (dd, J = 10.0, 2.0 Hz, 1H), 3.87 (s, 3H). |
| 13 | (400 MHz, DMSO-d₆) δ 12.35 (s, 1H), 11.90 (s, 1H), 8.58-8.56 (m, 1H), |
| | 8.36 (s, 1H), 8.28 (s, 1H), 8.09 (dd, J = 6.4, 1.6 Hz, 1H), 7.78-7.40 (m, |
| | 14H), 6.71 (t, J = 7.0 Hz, 1H). |
| 14 | (400 MHz, DMSO-d₆) δ 12.00 (s, 1H), 11.86 (s, 1H), 8.57 (d, J = 7.2 Hz, |
| | 1H), 8.34 (s, 1H), 8.09 (d, J = 6.4 Hz, 1H), 7.66-7.45 (m, 6H), 7.42 (t, J = |
| | 8.4 Hz, 2H), 7.30 (s, 1H), 7.20 (t, J = 8.0 Hz, 1H), 6.80-6.60 (m, 4H), 5.36 |
| | (s, 2H). |
| 16 | (300 MHz, DMSO-d₆) δ 12.60 (s, 1H), 11.97 (s, 1H), 8.97 (s, 1H), 8.59 (dd, |
| | J = 7.5, 2.1 Hz, 1H), 8.47-8.44 (m, 2H), 8.35 (s, 1H), 8.17 (d, J = 7.5 Hz, |
| | 1H), 8.11 (d, J = 6.3 Hz, 1H), 7.88 (s, 1H), 7.76 (d, J = 9.0 Hz, 2H), 7.64- |
| | 7.59 (m, 2H), 7.45-7.39 (m, 3H), 7.27 (d, J = 9.0 Hz, 2H), 6.72 (t, J = 7.5 |
| | Hz, 1H). |
| 17 | (300 MHz, DMSO-d₆) δ 11.62 (s, 1H), 11.97 (s, 1H), 8.61-8.58 (m, 2H), |
| | 8.34 (s, 1H), 8.12 (d, J = 7.2 Hz, 2H), 8.00 (s, 1H), 7.84-7.75 (m, 3H), |
| | 7.64-7.60 (m, 2H), 7.46-7.39 (m, 2H), 7.29 (d, J = 9.0 Hz, 2H), 7.25-7.23 |
| | (m, 1H), 6.72 (t, J = 7.2 Hz, 1H) |
| 18 | (300 MHz, CDCl₃) δ 11.88 (s, 1H), 8.52 (s, 1H), 8.76 (dd, J = 7.2, 2.1 Hz, |
| | 1H), 8.46 (s, 1H), 7.84 (d, J = 8.7 Hz, 2H), 7.61 (dd, J = 6.6, 2.1 Hz, 1H), |
| | 7.51-7.48 (m, 3H), 7.44-7.40 (m, 2H), 7.32-7.29 (m, 7H), 6.61 (t, J = 7.1 |
| | Hz, 1H). |
| 19 | (300 MHz, DMSO-d₆) δ 10.30 (s, 1H), 8.44 (s, 1H), 8.04 (s, 1H), 7.83 (d, J = |
| | 8.1 Hz, 1H), 7.69 (d, J = 9.0 Hz, 2H), 7.52 (s, 1H), 7.48-7.43 (m, 2H), 7.39- |
| | 7.33 (m, 2H), 7.17-7.10 (m, 3H), 6.81-6.80 (m, 1H), 6.62 (d, J = 8.4 Hz, |
| | 1H), 6.49 (d, J = 8.1 Hz, 1H), 5.20 (s,2H), 4.24 (dd, J = 14.1, 7.2 Hz, 2H), |
| | 3.87 (s, 3H), 1.29 (t, J = 7.2 Hz, 3H). |
| 20 | (400 MHz, DMSO-d₆) δ 10.31 (s, 1H), 10.06 (s, 1H), 8.46 (s, 1H), 8.13 (s, |
| | 1H), 7.94 (s, 1H), 7.83 (d, J = 8.0 Hz, 1H), 7.69-7.56 (m, 2H), 7.48-7.31 |
| | (m, 2H), 7.14 (d, J = 8.8 Hz, 2H), 6.49 (d, J = 8.0 Hz, 1H), 4.24 (dd, J = |
| | 14.0, 6.8 Hz, 2H), 3.87 (s,3H), 2.06 (s, 3H), 1.29 (t, J = 6.8 Hz, 3H). |
| 21 | (400 MHz, DMSO-d₆) δ 12.38 (s, 1H), 11.95 (s, 1H), 8.59 (dd, J = 7.5, 1.5 |
| | Hz, 1H), 8.29 (s, 1H), 8.11 (dd, J = 6.6, 1.5 Hz, 1H), 7.79-7.68 (m, 3H), |
| | 7.64-7.59 (m, 2H), 7.45-7.39 (m, 2H), 7.24-7.15 (m, 4H), 7.02 (d, J = 7.5 |
| | Hz, 1H), 6.72 (t, J = 6.6 Hz, 1H), 6.62 (d, J = 8.1 Hz, 1H), 2.86 (s, 6H). |
| 22 | (400 MHz, DMSO-d₆) δ 12.35 (s, 1H), 11.95 (s, 1H), 8.59 (d, J = 7.2 Hz, |
| | 1H), 8.37 (s, 1H), 8.11 (d, J = 5.1 Hz, 1H), 7.74 (d, J = 9.0 Hz, 2H), 7.64- |
| | 7.59 (m, 3H), 7.45-7.39 (m, 2H), 7.73 (d, J = 9.0 Hz, 2H), 7.07 (t, J = 7.8 |
| | Hz, 1H), 6.96 (s, 1H), 6.90 (d, J = 7.8 Hz, 1H), 6.72 (t, J = 7.2 Hz, 1H), 6.45 |
| | (d, J = 5.1 Hz, 1H), 5.56 (d, J = 4.8 Hz, 1H), 2.64 (d, J = 4.8 Hz, 3H). |
| 23 | (600 MHz, CDCl₃) δ 11.88 (s, 1H), 8.75 (dd, J = 7.5, 2.4 Hz, 1H), 8.53 (s, |
| | 1H), 7.80 (d, J = 9.0 Hz, 2H), 7.61 (dd, J = 7.2, 2.4 Hz, 1H), 7.41 (dd, J = |
| | 9.0, 4.8 Hz, 2H), 7.30-7.25 (m, 2H), 7.18-7.14 (m, 3H), 6.86 (ddd, J = 7.8, |
| | 1.8, 0.9 Hz, 1H), 6.80 (dd, J = 2.4, 1.8 Hz, 1H), 6.66 (ddd, J = 8.4, 2.4, 0.0 |
| | Hz, 1H), 6.61 (dd, J = 7.5, 7.2 Hz, 1H), 3.67 (s, 2H), 2.16 (s, 3H), 2.14 (s, |
| | 3H). |
| 25 | (400 MHz, DMSO-d₆) δ 11.96 (s, 1H), 10.08 (s, 1H), 8.57 (dd, J = 7.2, 2.0 |
| | Hz, 1H), 8.46 (s, 1H), 8.11 (dd, J = 6.8, 2.4 Hz, 1H), 8.04 (s, 1H), 7.71 (dd, |
| | J = 12.4, 9.2 Hz, 4H), 7.62-7.56 (m, 4H), 7.52 (s, 1 H), 7.42 (t, J = 8.8 Hz, |
| | 2H), 7.19 (d, J = 8.8 Hz, 2H), 6.71 (t, J = 6.8 Hz, 1H), 3.86 (s, 3H), 2.05 (s, |
| | 3H). |
| 28 | (400 MHz, DMSO-d₆) δ 11.95 (s, 1H), 8.57 (dd, J = 7.2, 1.6 Hz, 1H), 8.48 |
| | (s, 1H), 8.11 (dd, J = 6.8, 2.0 Hz, 1H), 8.07 (s, 1H), 7.72 (d, J = 9.2 Hz, 2H), |
| | 7.67 (s, 1H), 7.62-7.54 (m, 4H), 7.47-7.39 (m, 4H), 7.19 (d, J = 8.8 Hz, |
| | 2H), 6.71 (t, J = 7.2 Hz, 1H), 3.85 (t, J = 11.2 Hz, 4H), 3.40-3.34 (m, 3H). |
| 29 | (400 MHz, CDCl₃) δ 11.84 (s, 1H), 8.73 (dd, J = 7.6, 2.4 Hz, 1H), 8.45 (s, |
| | 1H), 7.75 (dd, J = 6.8, 2.0 Hz, 2H), 7.65 (s, 1H), 7.59 (dd, J = 6.8, 2.4 Hz, |
| | 1H), 7.42-7.38 (m, 2H), 7.31-7.24 (m, 3H), 7.08 (dd, J = 6.8, 2.0 Hz, 2H), |
| | 6.95 (d, J = 2.4 Hz, 1H), 6.92 (d, J = 7.6 Hz, 1H), 6.59 (t, J = 6.8 Hz, 1H), |
| | 3.89 (s, 1H), 2.93 (s, 6H). |
| 30 | (400 MHz, DMSO-d₆) δ 11.96 (s, 1H), 8.51 (dd, J = 7.5, 1.8 Hz, 1H), 8.43 |
| | (s, 1H), 8.07 (dd, J = 6.3, 1.8 Hz, 1H), 8.02 (s, 1H), 7.73 (d, J = 9.0, 2H), |
| | 7.61 (dd, J = 9.0, 5.1 Hz, 2H), 7.54 (s, 1H), 7.42 (t, J = 8.7 Hz, 2H), 7.28 (d, |
| | J = 8.4 Hz, 2H), 7.19 (d, J = 8.7 Hz, 2H), 6.71 (t, J = 6.6 Hz, 1H), 6.64 (d, |
| | J = 8.1 Hz, 2H), 5.29 (s, 2H), 3.86 (s, 3H). |
| 31 | (300 MHz, CDCl₃) δ 11.85 (s, 1H), 8.87 (s, 1H), 8.73 (dd, J = 7.2, 2.1 Hz, |
| | 1H), 8.66 (d, J = 3.6 Hz, 1H), 8.48 (s, 1H), 7.94 (d, J = 7.8 Hz, 1H), 7.76 (d, |
| | J = 9.0 Hz, 1H), 7.63-7.58 (m, 3H), 7.43-7.38 (m, 3H), 7.28-7.23 (m, 2H), |
| | 7.08 (d, J = 9.3 Hz, 2H), 6.59 (t, J = 6.6 Hz, 1H), 3.91 (s, 3H). |
| 32 | (400 MHz, CDCl₃) δ 11.84 (s, 1H), 8.74 (dd, J = 7.2, 2.0 Hz, 1H), 8.44 (s, |
| | 1H), 7.76 (dd, J = 6.8, 2.0 Hz, 2H), 7.68 (s, 1H), 7.63 (s, 1H), 7.60 (dd, J = |
| | 6.8, 2.4 Hz, 1H), 7.48 (d, J = 8.8, Hz, 2H), 7.42-7.39 (m, 2H), 7.26 (dd, J = |
| | 10.0, 2.4 Hz, 2H), 7.10 (dd, J = 6.8, 2.0 Hz, 2H), 6.98 (d, J = 8.8 Hz, 2H), |
| | 6.60 (t, J = 6.8 Hz, 1H), 3.90 (s, 3H), 3.24 (s, 4H), 3.07 (s, 4H). |
| 33 | (400 MHz, CDCl₃) δ 11.84 (s, 1H), 8.74 (dd, J = 7.2, 2.4 Hz, 1H), 8.46 (s, |
| | 1H), 7.74 (dd, J = 6.8, 2.0 Hz, 2H), 7.69 (s, 1H), 7.64 (s, 1H), 7.60 (dd, J = |
| | 6.8, 2.4 Hz, 1H), 7.42-7.38 (m, 2H), 7.33 (t, J = 8.0 Hz, 1H), 7.28-7.24 (m, |
| | 2H), 7.14 (d, J = 2.0 Hz, 1H), 7.08 (dd, J = 6.8, 2.4 Hz, 3H), 6.96 (dd, J = |
| | 8.4, 2.0 Hz, 1H), 6.60 (t, J = 6.8 Hz, 1H), 3.89 (s, 3H), 3.13 (s, 4H), 2.99 (s, |
| | 4H). |
| 34 | (400 MHz, CDCl₃) δ 11.84 (s, 1H), 8.74 (dd, J = 7.2, 2.0 Hz, 1H), 8.44 (s, |
| | 1H), 7.76 (d, J = 8.8, Hz, 2H), 7.68 (s, 1H), 7.62 (s, 1H), 7.59 (dd, J = 6.8, |
| | 1.6 Hz, 1H), 7.47 (d, J = 8.4 Hz, 2H), 7.42-7.39 (m, 2H), 7.26 (dd, J = 9.6, |
| | 9.2 Hz, 2H), 7.10 (d, J = 8.8 Hz, 2H), 6.98 (d, J = 8.8 Hz, 2H), 6.59 (t, J = |
| | 6.8 Hz, 1H), 3.90 (s, 3H), 3.30 (t, J = 4.8 Hz, 4H), 2.60 (t, J = 4.8 Hz, 4H), |
| | 2.36 (s, 3H). |
| 35 | (400 MHz, DMSO-d₆) δ 11.99 (s, 1H), 10.07 (s, 1H), 8.59 (dd, J = 6.8, 2.4 |
| | Hz, 1H), 8.49 (s, 1H), 8.11 (dd, J = 6.8, 2.4 Hz, 1H), 7.79-7.30 (m, 11H), |
| | 6.72 (t, J = 6.8 Hz, 1H), 2.06 (s, 3H). |
| 36 | (400 MHz, DMSO-d₆) δ 11.99 (s, 1H), 8.60 (dd, J = 7.2, 2.0 Hz, 1H), 8.47 |
| | (s, 1H), 8.11 (dd, J = 7.2, 2.0 Hz, 1H), 7.78 (d, J = 8.8 Hz, 2H), 7.64-7.59 |
| | (m, 2H), 7.43 (t, J = 8.8 Hz, 2H), 7.32 (d, J = 9.2 Hz, 2H), 7.27-7.23 (m, |
| | 2H), 7.12 (t, J = 7.6 Hz, 1H), 6.72 (t, J = 7.2 Hz, 1H), 6.63-6.60 (m, 1H), |
| | 5.19 (s, 2H). |
| 37 | (400 MHz, CDCl₃) δ 11.84 (s, 1H), 8.73 (dd, J = 7.6, 2.4 Hz, 1H), 8.46 (s, |
| | 1H), 7.75 (dd, J = 6.8, 2.0 Hz, 2H), 7.63 (s, 1H), 7.62 (s, 1H), 7.59 (dd, J = |
| | 6.8, 2.4 Hz, 1H), 7.57 (s, 1H), 7.50-7.47 (m, 1H), 7.42-7.38 (m, 3H), 7.34 |
| | (d, J = 7.6 Hz, 1H), 7.28-7.24 (m, 2H), 7.08 (dd, J = 6.8, 2.4 Hz, 2H), 6.59 |
| | (t, J = 7.2 Hz, 1H), 3.88 (s, 3H), 3.44 (s, 1H), 2.19 (s, 6H). |
| 38 | (300 MHz, CDCl₃) δ 11.84 (s, 1H), 8.74 (dd, J = 7.2, 1.8 ,Hz, 1H), 8.45 (s, |
| | 1H), 7.75 (d, J = 9.3, Hz, 2H), 7.70 (s, 1H), 7.63 (s, 1H), 7.59 (dd, J = 6.8, |
| | 2.1 Hz, 1H), 7.40 (dd, J = 9.0, 5.1 Hz, 2H), 7.35-7.23 (m, 3H), 7.16 (s, 1H), |
| | 7.08 (t, J = 4.5 Hz, 3H), 6.96 (d, J = 7.8 Hz, 1H), 6.59 (t, J = 6.9 Hz, 1H), |
| | 3.89 (s, 3H), 3.21 (t, J = 4.8 Hz, 4H), 2.53 (t, J=4.5 Hz, 4H), 2.31 (s, 3H). |
| 42 | (300 MHz, CDCl₃) δ 11.92 (s, 1H), 8.76 (dd, J = 7.2, 2.1 Hz, 1H), 8.51 (s, |
| | 1H), 7.85 (dd, J = 6.6, 4.8 Hz, 4H), 7.61 (dd, J = 6.6, 2.1 Hz, 1H), 7.50-7.39 |
| | (m, 5H), 7.29-7.21 (m, 3H), 6.93 (s, 1H) 6.61 (t, J = 7.2 Hz, 1H). |
| 43 | (400 MHz, DMSO-d₆) δ 11.98 (s, 1H), 8.56 (dd, J = 7.6, 2.4 Hz, 1H), 8.52 |
| | (s, 1H), 8.30 (s, 1H), 8.11 (dd, J = 6.4, 2.0 Hz, 1H), 7.77 (d, J = 9.2 Hz, 2H), |
| | 7.63-7.59 (m, 2H), 7.42 (t, J = 8.8 Hz, 2H), 7.30 (d, J = 9.2 Hz, 2H), 7.09 (t, |
| | J = 7.6 Hz, 1H), 6.97 (s, 1H), 6.92 (d, J = 7.6 Hz, 1H), 6.72 (t, J = 6.8 Hz, |
| | 1H), 6.59-6.56 (m, 1H), 5.17 (s, 2H). |
| 44 | (300 MHz, DMSO-d₆) δ 11.94 (s, 1H), 8.57 (dd, J = 7.2, 2.4 Hz, 1H), 8.47 |
| | (s, 1H), 8.08 (dd, J = 6.3, 2.1 Hz, 1H), 8.04 (s, 1H), 7.71 (d, J = 8.7 Hz, 2H), |
| | 7.62-7.58 (m, 3H), 7.51 (t, J = 3.6 Hz, 2H), 7.45-7.34 (m, 4H), 7.18 (d, J = |
| | 8.7 Hz, 2H), 6.71 (t, J = 6.9 Hz, 1H), 3.85 (s, 3H), 3.67 (s, 2H), 2.18 (s, 3H). |
| 45 | (400 MHz, DMSO-d₆) δ 11.99 (s, 1H), 8.59 (dd, J = 7.6, 2.4 Hz, 1H), 8.44 |
| | (s, 1H), 8.11 (dd, J = 6.4, 2.0 Hz, 1H), 8.01 (s, 1H), 7.79 (d, J = 9.2 Hz, 2H), |
| | 7.63-7.60 (m, 2H), 7.42 (t, J = 8.8 Hz, 2H), 7.29 (dd, J = 6.8, 2.0 Hz, 2H), |
| | 7.12 (s, 1H), 6.72 (t, J = 7.2 Hz, 1H), 3.92 (s, 3H). |
| 46 | (300 MHz, DMSO-d₆) δ 11.93 (s, 1H), 8.56 (dd, J = 7.5, 2.1 Hz, 1H), 8.43 |
| | (s, 1H), 8.08 (dd, J = 6.6, 2.1 Hz, 1H), 8.03 (s, 1H), 7.71 (d, J = 9.0 Hz, 2H), |
| | 7.61-7.56 (m, 2H), 7.52 (s, 1H), 7.40 (t, J = 8.7 Hz, 2H), 7.19-7.14 (m, 3H), |
| | 6.76 (d, J = 7.8 Hz, 2H), 6.69 (t, J = 7.2 Hz, 1H), 6.57 (d, J = 9.6 Hz, 1H), |
| | 5.74 (d, J = 5.1 Hz, 1H), 3.84 (s, 3H), 2.63 (d, J = 4.8 Hz, 3H). |
| 47 | (400 MHz, DMSO-d₆) δ 11.97 (s, 1H), 10.03 (s, 1H), 8.58 (dd, J = 7.2, 2.0 |
| | Hz, 1H), 8.55 (s, 1H), 8.38 (s, 1H), 8.13-8.09 (m, 2H), 7.76 (dd, J = 8.8, 6.8 |
| | Hz, 2H), 7.63-7.59 (m, 2H), 7.51-7.36 (m, 5H), 7.30 (dd, J = 6.8, 2.0 Hz, |
| | 2H), 6.72 (t, J = 6.8 Hz, 1H), 2.05 (s, 3H). |
| 48 | (400 MHz, DMSO-d₆) δ 11.96 (s, 1H), 8.58 (dd, J = 7.2, 2.0 Hz, 1H), 8.51 |
| | (s, 1H), 8.10 (dd, J = 6.4, 1.6 Hz, 1H), 7.74 (dd, J = 9.2, 7.2 Hz, 2H), 7.62- |
| | 7.59 (m, 2H), 7.44-7.39 (m, 2H), 7.24 (dd, J = 8.8, 6.8 Hz, 2H), 7.11 (t, J = |
| | 8.0 Hz, 1H), 6.87 (t, J= 1.6 Hz, 1H), 6.81 (d, J = 7.6 Hz, 1H), 6.71 (t, J = |
| | 6.8 Hz, 1H), 6.60 (dd, J = 8.0, 2.0 Hz, 1H), 5.23 (s, 2H). |
| 49 | (400 MHz, DMSO-d₆) δ 11.94 (s, 1H), 10.07 (s, 1H), 8.54 (dd, J = 7.6, 2.4 |
| | Hz, 1H), 8.52 (s, 1H), 8.07 (dd, J = 6.4, 2.0 Hz, 1H), 8.04 (s, 1H), 7.71 (t, |
| | J = 6.8 Hz, 2H), 7.60-7.53 (m, 3H), 7.38 (dd, J = 17.6, 8.8 Hz, 4H), 7.21 (d, |
| | J = 8.8 Hz, 2H), 6.69 (t, J = 7.2 Hz, 1H), 2.04 (s, 3H). |
| 50 | (400 MHz, DMSO-d₆) δ 11.95 (s, 1H), 10.03 (s, 1H), 8.58 (dd, J = 7.2, 2.0 |
| | Hz, 1H), 8.47 (s, 1H), 8.10 (dd, J = 6.4, 2.0 Hz, 1H), 7.94 (s, 1H), 7.73 (dd, |
| | J = 6.8, 2.0 Hz, 2H), 7.62-7.59 (m, 2H), 7.50 (d, J = 8.4 Hz, 1H), 7.44-7.36 |
| | (m, 3H), 7.31 (d, J = 7.6 Hz, 1H), 7.22 (dd, J = 6.8, 2.0 Hz, 2H), 6.71 (t, J = |
| | 7.2 Hz, 1H), 2.54 (s, 3H), 2.05 (s, 3H). |
| 51 | (400 MHz, DMSO-d₆) δ 11.86 (s, 1H), 8.52 (dd, J = 7.2, 2.4 Hz, 1H), 8.44 |
| | (s, 1H), 8.10 (dd, J = 6.8, 2.4 Hz, 1H), 7.74 (dd, J = 6.4, 2.0 Hz, 2H), 7.62- |
| | 7.59 (m, 2H), 7.43 (t, J = 8.8 Hz, 2H), 7.23 (dd, J = 6.8, 2.0 Hz, 2H), 7.09 (t, |
| | J = 15.6 Hz, 1H), 6.80 (t, J = 3.6 Hz, 1H), 6.72 (dd, J = 13.2, 6.4 Hz, 2H), |
| | 6.55 (dd, J= 8.0, 1.2 Hz, 1H), 5.16 (s, 2H), 2.52 (s, 3H). |
| 52 | (300 MHz, DMSO-d₆) δ 12.35 (s, 1H), 8.61 (dd, J = 7.2, 2.4 Hz, 1H), 8.48 |
| | (s, 1H), 8.34 (d, J = 9.0 Hz, 1H), 8.27 (d, J = 2.7 Hz, 1H), 8.13 (dd, J = 6.3, |
| | 1.8 Hz, 1H), 8.06 (s, 1H), 7.79 (dd, J = 9.0, 2.7 Hz, 1H), 7.63-7.59 (m, 2H), |
| | 7.54 (s, 1H), 7.42 (t, J = 18.0 Hz, 2H), 7.13 (t, J = 15.6 Hz, 1H), 6.83 (s, |
| | 1H), 6.74 (dd, J = 15.0, 7.8 Hz, 2H), 6.60 (t, J = 9.6 Hz, 1H), 5.21 (s, 2H), |
| | 3.86 (s, 3H). |
| 53 | (400 MHz, DMSO-d₆) δ 12.35 (s, 1H), 10.06 (s, 1H), 8.61 (dd, J = 7.6, 2.4 |
| | Hz, 1H), 8.50 (s, 1H), 8.33 (d, J = 9.2 Hz, 1H), 8.26 (d, J = 2.8 Hz, 1H), |
| | 8.14-8.12 (m, 2H), 7.95 (s, 1H), 7.78 (dd, J = 8.8, 2.8 Hz, 1H), 7.62-7.59 |
| | (m, 2H), 7.57 (s, 1H), 7.55 (s, 1H), 7.44-7.39 (m, 3H), 7.34 (d, J = 7.6 Hz, |
| | 1H), 6.72 (t, J = 14.0 Hz, 1H), 3.86 (s, 3H), 2.05 (s, 3H). |
| 54 | (400 MHz, DMSO-d₆) δ 12.00 (s, 1H), 8.59 (dd, J = 7.6, 2.4 Hz, 1H), 8.45 |
| | (s, 1H), 8.11 (dd, J = 6.8, 2.0 Hz, 1H), 8.04 (s, 1H), 7.74-7.70 (m, 2H), |
| | 7.61-7.52 (m, 6H), 7.22-7.18 (m, 2H), 7.13 (t, J = 8.0 Hz, 1H), 6.82-6.60 |
| | (m, 4H), 5.20 (s, 2H), 3.87 (s, 3H). |
| 55 | (400 MHz, DMSO-d₆) δ 11.90 (s, 1H), 8.59 (dd, J = 7.0, 2.2 Hz, 1H), 8.45 |
| | (s, 1H), 8.13 (dd, J = 7.0, 2.2 Hz, 1H), 8.04 (s, 1H), 7.75-7.38 (m, 7H), |
| | 7.22-7.18 (m, 2H), 7.13 (t, J = 7.6 Hz, 1H), 6.81 (t, J = 2.0 Hz, 1H), 6.76- |
| | 6.60 (m, 3H), 5.20 (s, 2H), 3.87 (s, 3H). |
| 56 | (400 MHz, DMSO-d₆) δ 11.99 (s, 1H), 10.06 (s, 1H), 8.58 (dd, J = 7.2, 2.4 |
| | Hz, 1H), 8.48 (s, 1H), 8.13-8.09 (m, 2H), 7.93 (s, 1H), 7.74-7.71 (m, 2H), |
| | 7.61-7.51 (m, 7H), 7.42 (t, J = 8.0 Hz, 1H), 7.34-7.17 (m, 3H), 6.72 (t, J = |
| | 6.8 Hz, 1H), 3.86 (s, 3H), 2.05 (s, 3H). |
| 57 | (400 MHz, DMSO-d₆) δ 11.91 (s, 1H), 10.06 (s, 1H), 8.58 (dd, J = 7.2, 2.0 |
| | Hz, 1H), 8.48 (s, 1H), 8.14-8.11 (m, 2H), 7.93 (s, 1H), 7.73 (d, J = 9.0 Hz, |
| | 2H), 7.66-7.31 (m, 8H), 7.19 (d, J = 9.0 Hz, 2H), 6.73 (t, J = 7.2 Hz, 1H), |
| | 3.86 (s, 3H), 2.05 (s, 3H). |
| 58 | (300 MHz, DMSO-d₆) δ 11.96 (s, 1H), 8.54 (dd, J = 7.5, 2.1 Hz, 1H), 8.50 |
| | (s, 1H), 8.10 (dd, J = 6.3, 2.1 Hz, 1H), 7.90 (dd, J = 3.0, 1.2 Hz, 1H), 7.73 |
| | (d, J = 8.7 Hz, 2H), 7.63-7.58 (m, 3H), 7.44-7.38 (m, 2H), 7.19 (d, J = 7.8 |
| | Hz, 2H), 7.13 (t, J = 7.5 Hz, 1H), 7.08 (dd, J = 4.5, 1.2 Hz, 1H), 6.78 (d, J = |
| | 1.8 Hz, 1H), 6.71 (t, J = 9.2 Hz, 2H), 6.62 (dd, J = 8.1, 1.2 Hz, 1H), 5.20 (s, |
| | 2H). |
| 59 | (400 MHz, DMSO-d₆) δ 11.95 (s, 1H), 10.05 (s, 1H), 8.57 (dd, J = 7.2, 2.0 |
| | Hz, 1H), 8.52 (s, 1H), 8.10 (dd, J = 6.4, 2.0 Hz, 1H), 7.93 (dd, J = 2.8, 1.2 |
| | Hz, 1H), 7.88 (s, 1H), 7.72 (d, J = 6.8 Hz, 2H), 7.64-7.59 (m, 4H), 7.44- |
| | 7.39 (m, 3H), 7.30 (d, J = 7.6 Hz, 1H), 7.17 (dd, J = 7.2, 2.4 Hz, 2H), 7.05 |
| | (dd, J = 5.2, 1.2 Hz, 1H), 6.71 (t, J = 7.2 Hz, 1H), 2.03 (s, 3H). |
| 60 | (300 MHz, DMSO-d₆) δ 11.95 (s, 1H), 9.85 (s, 1H), 8.58 (dd, J = 7.5, 2.1 |
| | Hz, 1H), 8.48 (s, 1H), 8.10 (dd, J = 6.6, 2.4 Hz, 1H), 8.07 (s, 1H), 7.73 (d, |
| | J = 8.7 Hz, 2H), 7.60 (dd, J = 9.0, 5.1 Hz, 3H), 7.49-7.39 (m, 5H), 7.26 (t, J = |
| | 5.4 Hz, 1H), 7.19 (d, J = 7.5 Hz, 2H), 6.71 (t, J = 6.6 Hz, 1H), 3.86 (s, 3H), |
| | 2.93 (s, 3H). |
| 61 | (300 MHz, CDCl₃) δ 11.40 (s, 1H), 9.30 (s, 1H), 8.44 (s, 1H), 7.73-7.62 (m, |
| | 8H), 7.26-7.21 (m, 2H), 7.44 (s, 1H), 7.09 (d, J = 8.7 Hz, 2H), 6.95 (d, J = |
| | 8.1 Hz, 1H), 6.89 (s, 1H), 6.75 (d, J = 8.1 Hz, 1H), 3.90 (s, 3H), 2.63-2.54 |
| | (m, 4H), 2.12-2.08 (m, 2H). |
| 62 | (300 MHz, CDCl₃) δ 11.37 (s, 1H), 9.28 (s, 1H), 8.44 (s, 1H), 7.90-7.62 (m, |
| | 9H), 7.26-7.21 (m, 2H), 7.53 (d, J = 8.1 Hz, 1H), 7.39 (dd, J = 8.1, 8.1 Hz, |
| | 1H), 7.32-7.27 (m, 2H), 7.05 (d, J = 9.0 Hz, 2H), 3.88 (s, 3H), 2.61-2.53 |
| | (m, 4H), 2.15 (s, 3H), 2.11-2.07 (m, 2H). |
| 63 | (400 MHz, DMSO-d₆) δ 11.90 (s, 1H), 8.48 (d, J = 7.6 Hz, 1H), 8.44 (s, |
| | 1H), 8.04 (s, 1H), 7.71 (dd, J = 7.2, 2.4 Hz, 2H), 7.52-7.45 (m, 3H), 7.43 (t, |
| | J = 6.4 Hz, 2H), 7.17 (dd, J = 7.2, 2.4 Hz, 2H), 7.12 (t, J = 7.6 Hz, 1H), 6.80 |
| | (t, J = 2.0 Hz, 1H), 6.74 (d, J = 7.6 Hz, 1H), 6.69 (d, J = 8.0 Hz, 1H), 6.60 |
| | (dd, J = 8.0, 1.2 Hz, 1H), 5.19 (s, 2H), 3.86 (s, 3H), 2.06 (s, 3H). |
| 64 | (400 MHz, DMSO-d₆) δ 8.44 (s, 1H), 8.07 (s, 1H), 6.73 (t, J = 3.6 Hz, 1H), |
| | 7.61 (s, 1H), 7.58 (s, 1H), 7.53-7.44 (m, 4H), 7.38-7.10 (m, 8H), 6.24 (s, |
| | 1H), 3.85 (s, 3H), 3.30 (s, 3H), 3.16 (s, 3H), 2.84 (s, 3H). |
| 65 | (400 MHz, DMSO-d₆) δ 11.95 (s, 1H), 8.57 (dd, J = 9.6, 2.0 Hz, 1H), 8.48 |
| | (s, 1H), 8.09 (dd, J = 6.8, 2.4 Hz, 1H), 8.06 (s, 1H), 7.73-7.71 (m, 3H), |
| | 7.64-7.59 (m, 4H), 7.54 (t, J = 8.0 Hz, 1H), 7.46 (t, J = 8.8 Hz, 1H), 7.40 (t, |
| | J = 6.4 Hz, 2H), 7.20 (d, J = 8.8 Hz, 2H), 6.71 (t, J = 7.2 Hz, 1H), 3.83 (s, |
| | 3H), 3.22 (s, 3H), 2.91 (s, 3H). |
| 66 | (400 MHz, DMSO-d₆) δ 12.23 (s, 1H), 8.46 (dd, J = 3.2, 2.0 Hz, 1H), 8.46 |
| | (s, 1H), 8.16 (dd, J = 6.4, 2.0 Hz, 1H), 8.05 (s, 1H), 7.74 (d, J = 8.8 Hz, 2H), |
| | 7.52 (s, 1H), 7.21 (d, J = 9.2 Hz, 2H), 7.13 (t, J = 7.6 Hz, 1H), 6.82 (t, J = |
| | 1.6 Hz, 1H), 6.75 (td, J = 7.6, 1.2 Hz, 1H), 6.64-6.59 (m, 2H), 5.21 (s, 2H), |
| | 3.87 (s, 3H), 3.64 (s, 3H). |
| 67 | (400 MHz, DMSO-d₆) δ 13.31 (s, 1H), 11.96 (s, 1H), 10.01 (s, 1H), 8.58 |
| | (dd, J = 7.4, 2.2 Hz, 1H), 8.53 (s, 1H), 8.11 (dd, J = 6.6, 2.2 Hz, 1H), 7.90 |
| | (s, 1H), 7.83-7.82 (m, 1H), 7.74-7.72 (m, 2H), 7.63-7.59 (m, 3H), 7.44- |
| | 7.33 (m, 4H), 7.20 (d, J = 9.2 Hz, 2H), 6.72 (t, J = 7.0 Hz, 1H), 6.35 (t, J = |
| | 2.0 Hz, 1H), 2.03(s, 3H). |
| 68 | (300 MHz, CDCl₃) δ 11.32 (s, 1H), 9.26 (s, 1H), 8.44 (s, 1H), 7.72-7.69 (m, |
| | 3H), 7.60 (s, 1H), 7.37-7.20 (m, 5H), 7.08 (d, J = 9.0 Hz, 2H), 6.94 (d, J = |
| | 7.5 Hz, 1H), 6.88 (s, 1H), 6.72 (d, J = 7.8, 1.2 Hz, 1H), 3.89 (s, 3H), 2.60- |
| | 2.53 (m, 4H), 2.12-2.08 (m, 2H). |
| 69 | (300 MHz, CDCl₃) δ 11.31 (s, 1H), 9.24 (s, 1H), 8.43 (s, 1H), 7.75-7.63 (m, |
| | 5H), 7.54 (d, J = 8.1 Hz, 1H), 7.41-7.27 (m, 6H), 7.03 (d, J = 8.7 Hz, 2H), |
| | 3.88 (s, 3H), 2.61-2.53 (m, 4H), 2.15 (s, 3H), 2.12-2.07 (m, 2H). |
| 70 | (400 MHz, DMSO-d₆) δ 11.90 (s, 1H), 10.05 (s, 1H), 8.49 (s, 1H), 8.47 (s, |
| | 1H), 8.12 (s, 1H), 7.92 (s, 1H), 7.70 (dd, J = 7.2, 2.0 Hz, 2H), 7.56 (d, J = |
| | 7.6 Hz, 2H), 7.51-7.48 (m, 2H), 7.42 (dd, J = 17.2, 8.8 Hz, 3H), 7.32 (d, J = |
| | 7.6 Hz, 1H), 7.16 (dd, J = 6.8, 2.4 Hz, 2H), 6.69 (d, J = 8.0 Hz, 1H), 3.86 (s, |
| | 3H), 2.06 (s, 3H), 2.04 (s, 3H). |
| 71 | (400 MHz, DMSO-d₆) δ 10.66 (s, 1H), 8.44 (s, 1H), 8.04 (s, 1H), 7.72-7.69 |
| | (m, 3H), 7.52-7.48 (m, 3H), 7.37 (t, J = 8.8 Hz, 2H), 7.17 (d, J = 8.8 Hz, |
| | 2H), 7.13 (t, J = 8.0 Hz, 1H), 6.82-6.81 (m, 1H), 6.75-6.73 (m, 1H), 6.62 |
| | (ddd, J = 8.0, 2.4, 0.8 Hz, 1H), 6.36 (d, J = 7.2 Hz, 1H), 5.20 (s, 2H), 3.87 |
| | (s, 3H), 2.30 (s, 3H). |
| 72 | (400 MHz, DMSO-d₆) δ 10.65 (s, 1H), 10.07 (s, 1H), 8.41 (s, 1H), 8.13 (s, |
| | 1H), 7.94-7.93 (m, 1H), 7.72-7.67 (m, 3H), 7.58-7.56 (m, 2H), 7.51-7.46 |
| | (m, 2H), 7.44-7.32 (m, 4H), 7.16 (d, J = 9.2 Hz, 2H), 6.36 (d, J = 7.2 Hz, |
| | 1H), 3.86 (s, 3H), 2.29 (s, 3H), 2.05 (s, 3H). |
| 73 | (400 MHz, DMSO-d₆) δ 11.86 (s, 1H), 8.76 (dd, J = 7.4, 2.2 Hz, 1H), 8.46 |
| | (s, 1H), 7.78-7.74(m, 2H), 7.69 (s, 1H), 7.61(s, 1H), 7.49 (dd, J = 6.6, 2.2 |
| | Hz, 1H), 7.26-7.21 (m, 2H), 7.19-7.06 (m, 4H), 7.00-6.95 (m, 1H), 6.89- |
| | 6.88 (m, 1H), 6.73 (dd, J = 8.0, 2.4 Hz, 1H), 6.61 (t, J = 7.0 Hz, 1H), 3.90 |
| | (s, 3H), 3.72 (s, 2H), 2.17(s, 3H). |
| 74 | (400 MHz, DMSO-d₆) δ 11.94 (s, 1H), 10.08 (s, 1H), 8.61 (dd, J = 7.2, 2.0 |
| | Hz, 1H), 8.47 (s, 1H), 8.13 (s, 1H), 8.02 (dd, J = 6.6, 2.2 Hz, 1H), 7.92 (s, |
| | 1H), 7.72 (d, J = 8.8 Hz, 2H), 7.58-7.56 (m, 2H), 7.48-7.24 (m, 5H), 7.18 |
| | (d, J = 8.8 Hz, 2H), 6.74 (t, J = 7.0 Hz, 1H), 3.86 (s, 3H), 2.08-2.05 (m, 6H). |
| 75 | (400 MHz, DMSO-d₆) δ 12.23 (s, 1H), 10.07 (s, 1H), 8.48 (s, 1H), 8.45 (dd, |
| | J = 7.2, 1.6 Hz, 1H), 8.17 (dd, J = 6.4, 2.0 Hz, 1H), 8.13 (s, 1H), 7.93 (s, |
| | 1H), 7.73 (d, J = 8.8 Hz, 2H), 7.58-7.56 (m, 2H), 7.42 (t, J = 7.6 Hz, 1H), |
| | 7.34 (d, J = 7.6 Hz, 1H), 7.20 (d, J = 8.8 Hz, 2H), 6.61 (t, J = 6.8 Hz, 1H), |
| | 3.87 (s, 3H), 3.64 (s, 3H), 2.05 (s, 3H). |
| 76 | (400 MHz, DMSO-d₆) δ 12.73 (s, 1H), 11.99 (s, 1H), 8.60 (dd, J= 7.0, 2.0 |
| | Hz, 1H), 8.31 (s, 1H), 8.12 (dd, J = 7.0, 2.0 Hz, 1H), 8.00-7.30 (m, 11H), |
| | 7.30-7.29 (m, 2H), 7.07 (s, 1H), 6.73 (t, J = 7.0 Hz, 1H). |
| 77 | (400 MHz, DMSO-d₆) δ 12.76 (s, 1H), 12.23 (s, 1H), 8.50-8.44 (m, 2H), |
| | 8.05 (s, 1H), 7.82 (s, 1H), 7.72 (d, J = 7.0 Hz, 2H), 7.52 (s, 1H), 7.21 (d, J = |
| | 7.0 Hz, 2H), 7.13 (t, J = 7.6 Hz, 1H), 6.82 (t, J = 1.6 Hz, 1H), 6.75 (d, J = |
| | 7.2 Hz, 1H), 6.63-6.55 (m, 2H), 5.22 (s, 2H), 3.87 (s, 3H). |
| 78 | (400 MHz, DMSO-d₆) δ 12.49 (s, 1H), 11.97 (s, 1H), 8.60 (dd, J = 7.3, 2.2 |
| | Hz, 1H), 8.24 (s, 1H), 8.19 (s, 1H), 8.12 (dd, J = 6.6, 2.2 Hz, 1H), 7.99 (d, |
| | J = 0.8 Hz, 1H), 7.81-7.71 (m, 2H), 7.67-7.57 (m, 2H), 7.49-7.37 (m, 2H), |
| | 7.29-7.18 (m, 2H), 6.72 (dd, J = 7.3, 6.6 Hz, 1H), 6.67 (s, 1H), 3.89 (s, 3H). |
| 79 | (400 MHz, DMSO-d₆) δ 12.66 (s, 1H), 11.98 (s, 1H), 8.60 (dd, J = 7.3, 2.2 |
| | Hz, 1H), 8.29 (s, 1H), 8.12 (dd, J = 6.6, 2.2 Hz, 1H), 8.09-8.04 (m, 1H), |
| | 7.80-7.76 (m, 2H), 7.75-7.66 (m, 2H), 7.62 (dd, J = 9.0, 5.0 Hz, 2H), 7.43 |
| | (t, J = 8.7 Hz, 2H), 7.28-7.23 (m, 2H), 6.96 (s, 1H), 6.73 (t, J = 7.3 Hz, 1H). |
| 82 | (400 MHz, CDCl₃) δ 11.86 (s, 1H), 10.43 (s, 1H), 8.75 (dd, J = 7.2, 2.4 Hz, |
| | 1H), 8.45 (s, 1H), 7.80 (d, J = 8.8 Hz, 2H), 7.73 (s, 1H), 7.65-7.59 (m, 2H), |
| | 7.46-7.34 (m, 5H), 7.29-7.24 (m, 2H), 7.20 (d, J = 9.2 Hz, 2H), 6.60 (t, J = |
| | 7.2 Hz, 1H), 3.49 (s, 2H), 2.24 (s, 6H). |
| 83 | (400 MHz, CDCl₃) δ 12.17 (s, 1H), 8.59 (dd, J = 7.2, 2.4 Hz, 1H), 8.47 (s, |
| | 1H), 7.80 (d, J = 8.8 Hz, 2H), 7.70 (s, 1H), 7.63 (dd, J =6.6, 2.4 Hz, 1H), |
| | 7.61 (s.1H), 7.26-7.22 (m, 1H), 7.12 (d, J = 9.2 Hz, 2H), 6.98-6.95 (m, 1H), |
| | 6.89 (s, 1H), 6.75-6.72 (m, 1H), 6.53 (t, J = 7.2 Hz, 1H), 5.03-4.98 (m, 1H), |
| | 3.09 (s, 3H), 3.72 (s, 2H), 2.03-1.95 (m, 4H), 1.83-1.80 (m, 1H), 1.55-1.49 |
| | (m, 4H), 1.25-1.22 (m, 1H). |
| 84 | (400 MHz, CDCl₃) δ 12.18 (s, 1H), 8.59 (dd, J = 7.2, 2.0 Hz, 1H), 8.47 (s, |
| | 1H), 7.79 (d, J = 9.2 Hz, 2H), 7.64-7.62 (m, 3H), 7.58 (s, 1H), 7.51-7.48 |
| | (m, 1H), 7.41 (t, J = 7.6 Hz, 1H), 7.37-7.34 (m, 1H), 7.10 (d, J = 9.2 Hz, |
| | 2H), 6.53 (t, J = 7.2 Hz, 1H), 5.03-4.97 (m, 1H), 3.89 (s, 3H), 3.46 (s, 2H), |
| | 2.20 (s, 6H), 2.02-1.94 (m, 4H), 1.83-1.79 (m, 1H), 1.56-1.46 (m, 4H), |
| | 1.28-1.25 (m, 1H). |
| 85 | (400 MHz, CDCl₃) δ 11.86 (s, 1H), 8.76-8.74 (m, 1H), 8.53 (s, 1H), 7.77 (d, |
| | J = 9.2 Hz, 2H), 7.61-7.60 (m, 2H), 7.43-7.39 (m, 2H), 7.29-7.25 (m, 3H), |
| | 7.12 (d, J= 8.8 Hz, 2H), 7.00 (d, J = 7.6 Hz, 2H), 6.92 (s, 1H), 6.76 (d, J = |
| | 8.0 Hz, 2H), 6.62-6.59 (m,2H). |
| 86 | (400 MHz, CDCl₃) δ 12.26 (s, 1H), 8.62 (dd, J =7.2, 2.4 Hz, 1H), 8.49 (s, |
| | 1H), 7.89-7.86 (m, 3H), 7.81 (s, 1H), 7.65 (dd, J = 6.8, 2.4 Hz, 1H), 7.22 (d, |
| | J = 8.8 Hz, 2H), 6.56-6.53 (m, 2H), 5.05-4.99 (m, 1H), 3.98 (s, 3H), 2.05- |
| | 1.95 (m, 4H), 1.84-1.80 (m, 1H), 1.53-1.48 (m, 4H), 1.26-1.24 (m, 1H). |
| 87 | (400 MHz, CDCl₃) δ 11.84 (s, 1H), 8.73 (dd, J = 7.2, 2.4 Hz, 1H), 8.46 (s, |
| | 1H), 7.75 (d, J = 9.2 Hz, 2H), 7.62-7.56 (m, 5H), 7.44-7.39 (m, 4H), 7.28- |
| | 7.24 (m, 2H), 7.09 (d, J = 9.2 Hz, 2H), 6.60 (t, J = 7.2 Hz, 1H), 3.90 (s, 3H), |
| | 3.57 (s, 2H), 2.34 (s, 6H). |
| 88 | (400 MHz, DMSO-d₆) δ 11.99 (s, 1H), 8.59 (dd, J = 7.3, 2.2 Hz, 1H), 8.49 |
| | (s, 1H), 8.12 (dd, J = 6.6, 2.2 Hz, 1H), 8.08-8.06 (m, 1H), 8.04-7.99 (m, |
| | 1H), 7.82-7.73 (m, 2H), 7.66-7.58 (m, 2H), 7.48-7.39 (m, 3H), 7.36-7.28 |
| | (m, 3H), 6.72 (t, J = 7.2 Hz, 1H), 3.43 (s, 2H), 2.11 (s, 6H). |
| 89 | (400 MHz, CDCl₃) δ 11.85 (s, 1H), 8.74 (dd, J = 7.3, 2.2 Hz, 1H), 8.46 (s, |
| | 1H), 7.79-7.73 (m, 2H), 7.64 (s, 1H), 7.63-7.58 (m, 2H), 7.55 (d, J = 8.3 |
| | Hz, 2H), 7.44-7.38 (m, 4H), 7.30-7.23 (m, 1H), 7.12-7.06 (m, 2H), 6.60 |
| | (dd, J = 7.3, 6.6 Hz, 1H), 3.95-3.88 (m, 3H), 3.57 (s, 2H), 2.67-2.29 (m, |
| | 11H). |
| 90 | (400 MHz, DMSO-d₆) δ 11.94 (s, 1H), 8.61 (dd, J = 7.3, 2.2 Hz, 1H), 8.48 |
| | (s, 1H), 8.06-8.00 (m, 2H), 7.75-7.69 (m, 2H), 7.60-7.50 (m, 3H), 7.50- |
| | 7.41 (m, 2H), 7.37-7.32 (m, 2H), 7.28-7.22 (m, 1H), 7.21-7.14 (m, 2H), |
| | 6.74 (dd, J = 7.2, 6.6 Hz, 1H), 3.85 (s, 3H), 3.40 (s, 2H), 2.08 (s, 9H). |
| 91 | (400 MHz, DMSO-d₆) δ 11.94 (s, 1H), 8.57 (dd, J = 7.3, 2.1 Hz, 1H), 8.47 |
| | (s, 1H), 8.10 (dd, J = 6.7, 2.2 Hz, 1H), 7.86 (s, 1H), 7.70 (d, J = 9.1 Hz, 2H), |
| | 7.60 (dd, J = 8.8, 5.0 Hz, 3H), 7.54-7.31 (m, 5H), 7.14 (s, 1H), 7.03 (d, J = |
| | 9.0 Hz, 2H), 6.75-6.67 (m, 1H), 3.83 (s, 3H), 3.33 (s, 2H), 1.97 (s, 6H). |
| 92 | (400 MHz, DMSO-d₆) δ 11.77 (s, 1H), 8.62 (dd, J = 7.0, 2.2 Hz, 1H), 8.46 |
| | (s, 1H), 8.15 (dd, J = 7.0, 2.2 Hz, 1H), 8.05 (s, 1H), 7.80-7.70 (m, 3H), 7.62 |
| | (td, J = 9.6, 2.8 Hz, 1H), 7.52 (s, 1H), 7.37-7.30 (m, 1H), 7.20 (d, J = 8.8 |
| | Hz, 2H), 7.13 (t, J = 7.8 Hz, 1H), 6.84-6.72 (m, 3H), 6.60 (d, J = 5.6 Hz, |
| | 1H), 5.21 (s, 2H), 3.87 (s, 3H). |
| 93 | (400 MHz, DMSO-d₆) δ 11.77 (s, 1H), 8.62 (dd, J = 7.4, 2.2 Hz, 1H), 8.49 |
| | (s, 1H), 8.16 (dd, J = 6.6, 2.2 Hz, 1H), 8.05 (s, 1H), 7.80-7.71 (m, 3H), |
| | 7.67-7.55 (m, 3H), 7.55-7.46 (m, 2H), 7.41-7.33 (m, 2H), 7.19 (d, J = 8.8 |
| | Hz, 2H), 6.78 (t, J = 7.0 Hz, 1H), 3.86 (s, 3H), 3.57 (s, 2H), 2.19 (s, 6H). |
| 96 | (400 MHz, DMSO-d₆) δ 11.89 (s, 1H), 8.58 (dd, J = 7.1, 2.2 Hz, 1H), 8.45 |
| | (s, 1H), 8.13 (dd, J = 7.1, 2.2 Hz, 1H), 8.05 (s, 1H), 7.97-7.95 (m, 1H), 7.74 |
| | (d, J = 9.0 Hz, 2H), 7.68-7.61 (m, 2H), 7.52 (s, 1H), 7.20 (d, J = 9.0 Hz, |
| | 2H), 7.13 (t, J = 8.0 Hz, 1H), 6.81 (t, J = 2.0 Hz, 1H), 6.76-6.71 (m, 2H), |
| | 6.62-6.60 (m, 1H), 5.21 (s, 2H), 3.86 (s, 3H). |
| 97 | (400 MHz, DMSO-d₆) δ 11.89 (s, 1H), 8.58 (dd, J = 7, 2.1 Hz, 1H), 8.48 (s, |
| | 1H), 8.13 (dd, J = 7.0, 2.1 Hz, 1H), 8.04 (s, 1H), 7.97-7.95 (m, 1H), 7.72 (d, |
| | J = 9.2 Hz, 2H), 7.68-7.60 (m, 2H), 7.58 (s, 1H), 7.56-7.53 (m, 1H), 7.51 |
| | (s, 1H), 7.45 (t, J = 7.6 Hz, 1H), 7.35-7.32 (m, 1H), 7.18 (d, J = 9.2 Hz, |
| | 2H), 6.72 (t, J =7.0 Hz, 1H), 3.85 (s, 3H), 3.41 (s, 2H), 2.09 (s, 6H). |
| 98 | (400 MHz, DMSO-d₆) δ 11.94 (s, 1H), 8.61 (dd, J = 7.3, 2.2 Hz, 1H), 8.48 |
| | (s, 1H), 8.06-8.00 (m, 2H), 7.75-7.69 (m, 2H), 7.60-7.50 (m, 3H), 7.50- |
| | 7.41 (m, 2H), 7.37-7.32 (m, 2H), 7.28-7.22 (m, 1H), 7.21-7.14 (m, 2H), |
| | 6.74 (dd, J = 7.2, 6.6 Hz, 1H), 3.85 (s, 3H), 3.40 (s, 2H), 2.08 (s, 9H). |
| 115 | (400 MHz, DMSO-d₆) δ 10.86 (s, 1H), 8.46 (s, 1H), 8.06-8.03 (m, 3H), 7.83 |
| | (d, J = 8.8 Hz, 2H), 7.53 (s, 1H), 7.50 (s, 1H), 7.43 (dd, J = 8.8, 8.8 Hz, 2H), |
| | 7.24 (d, J = 8.8 Hz, 2H), 7.14 (dd, J = 7.6, 7.6 Hz, 1H), 6.83 (dd, J = 2.0, 2.0 |
| | Hz, 1H), 6.76 (d, J = 7.6 Hz, 1H), 6.63 (dd, J = 8.4, 1.6 Hz, 1H), 5.21 (s, |
| | 2H), 3.87 (s, 3H). |
| 116 | (400 MHz, DMSO-d₆) δ 10.85 (s, 1H), 10.10 (s, 1H), 8.48 (s, 1H), 8.13 (s, |
| | 1H), 8.07-8.03 (m, 2H), 7.94 (s, 1H), 7.82 (d, J = 8.8 Hz, 2H), 7.58-7.56 |
| | (m, 2H), 7.50 (s, 1H), 7.46-7.41 (m, 3H), 7.34 (d, J = 7.6 Hz, 1H), 7.23 (d, |
| | J = 9.2 Hz, 2H), 3.87 (s, 3H), 2.06 (s, 3H). |
| 117 | (400 MHz, DMSO-d₆) δ 10.26 (s, 1H), 8.47 (s, 1H), 8.06-8.01 (m, 3H), 7.78 |
| | (d, J = 8.8 Hz, 2H), 7.53 (s, 1H), 7.41 (d, J = 3.6 Hz, 1H), 7.36 (t, J = 8.8 |
| | Hz, 2H), 7.24-7.12 (m, 4H), 6.83 (t, J = 2.0 Hz, 1H), 6.77-6.75 (m, 1H), |
| | 6.64-6.61 (m, 1H), 5.21 (s, 2H), 3.87 (s, 3H). |
| 118 | (400 MHz, DMSO-d₆) δ 10.25 (s, 1H), 10.07 (s, 1H), 8.49 (s, 1H), 8.13 (s, |
| | 1H), 8.06-8.02 (m, 2H), 7.94 (s, 1H), 7.80-7.50 (m, 2H), 7.60-7.56 (m, |
| | 2H), 7.45-7.33 (m, 5H), 7.24-7.20 (m, 2H), 7.17 (d, J = 3.6 Hz, 1H), 3.87 |
| | (s, 3H), 2.06 (s, 3H). |
| 119 | (400 MHz, DMSO-d₆) δ 10.29 (s, 1H), 8.47 (s, 1H), 8.05 (s, 1H), 7.92-7.70 |
| | (m, 4H), 7.58-7.52 (m, 2H), 7.41 (d, J = 3.6 Hz, 1H), 7.30 (d, J = 3.6 Hz, |
| | 1H), 7.25-7.21 (m, 3H), 7.14 (t, J = 7.6 Hz, 1H), 6.83-6.82 (m, 1H), 6.76 |
| | (d, J = 7.6 Hz, 1H), 6.62 (dd, J = 8.0, 2.0 Hz, 1H), 5.21 (s, 2H), 3.87 (s, 3H). |
| 120 | (400 MHz, DMSO-d₆) δ 10.29 (s, 1H), 10.07 (s, 1H), 8.50 (s, 1H), 8.13 (s, |
| | 1H), 7.94 (s, 1H), 7.90 (d, J = 9.6 Hz, 1H), 7.82 (d, J = 8.0 Hz, 1H), 7.78 (d, |
| | J = 8.8 Hz, 2H), 7.59-7.52 (m, 3H), 7.46-7.28 (m, 3H), 7.26 (d, J = 2.4 Hz, |
| | 1H), 7.24-7.21 (m, 3H), 3.87 (s, 3H), 2.06 (s, 3H) |
| 123 | (400 MHz, DMSO-d₆) δ 10.33 (s, 1H), 8.59 (s, 1H), 8.47 (s, 1H), 8.03 (s, |
| | 1H), 7.62 (dd, J = 7.2, 2.4 Hz, 2H), 7.58-7.53 (m, 4H), 7.51(s, 1H), 7.44 |
| | (dd, J = 16.0, 8.4 Hz, 3H), 7.34 (d, J = 7.6 Hz, 1H), 7.15 (dd, J = 6.8, 2.0 |
| | Hz, 2H), 3.85 (s, 3H), 3.46 (s, 3H), 3.42 (s, 2H), 2.10 (s, 6H). |
| 124 | (400 MHz, CDCl₃) δ 10.24 (s, 1H), 8.34 (s, 1H), 8.15 (s, 1H), 7.70 (d, J = |
| | 8.8 Hz, 2H), 7.37-7.34 (m, 2H), 7.31 (s, 1H), 7.27-7.23 (m, 2H), 7.19-7.11 |
| | (m, 4H), 7.07 (s, 1H), 6.63 (d, J = 7.2 Hz, 1H), 3.44 (s, 3H). |
| 125 | (400 MHz, DMSO-d₆) δ 12.44 (s, 1H), 10.34 (s, 1H), 9.95 (s, 1H), 8.61 (s, |
| | 1H), 8.31 (s, 1H), 8.03 (s, 1H), 7.65-7.62 (m, 3H), 7.59-7.56 (m, 2H), 7.47- |
| | 7.40 (m, 4H), 7.29 (t, J = 8.0 Hz, 1H), 7.23 (d, J = 8.8 Hz, 2H), 3.47 (s, 3H), |
| | 2.05 (s, 3H). |
| 126 | (400 MHz, CDCl₃) δ 10.22 (s, 1H), 8.46 (s, 1H), 8.10 (s, 1H), 7.71 (dd, J = |
| | 6.8, 2.0 Hz, 2H), 7.63 (s, 1H), 7.62 (s, 1H), 7.58 (s, 1H), 7.50-7.47 (m, 1H), |
| | 7.42-7.34 (m, 4H), 7.29-7.25 (m, 2H), 7.08 (dd, J = 6.8, 2.0 Hz, 2H), 3.90 |
| | (s, 3H), 3.54 (s, 2H), 3.45 (s, 3H), 2.60-2.26 (m, 8H), 2.23 (s, 3H). |
| 127 | (400 MHz, CDCl₃) δ 10.23 (s, 1H), 8.46 (s, 1H), 8.11 (s, 1H), 7.71 (dd, J = |
| | 6.8, 2.0 Hz, 2H), 7.65 (s, 1H), 7.60 (s, 1H), 7.58 (s, 1H), 7.52-7.48 (m, 1H), |
| | 7.44-7.34 (m, 4H), 7.30-7.23 (m, 2H), 7.07 (dd, J = 6.8, 2.0 Hz, 2H), 3.91 |
| | (s, 3H), 3.61-3.55 (m, 4H), 3.53 (s, 2H), 3.45 (s, 3H), 2.46-2.37 (m, 4H). |
| 128 | (600 MHz, CDCl₃) δ 10.09 (s, 1H), 8.46 (s, 1H), 8.18 (s, 1H), 7.84 (dd, J = |
| | 9.0, 0.6 Hz, 2H), 7.71-7.67 (m, 3H), 7.61 (s, 1H), 7.53 (dd, J = 9.0, 0.6 Hz, |
| | 2H), 7.24 (ddd, J = 7.2, 7.2, 0.6 Hz, 1H), 7.10 (dd, J = 6.6, 2.4 Hz, 2H), 6.96 |
| | (ddd, J = 7.2, 1.8, 1.2 Hz, 1H), 6.89 (ddd, J = 1.8, 1.8, 0.6 Hz, 1H), 6.73 |
| | (ddd, J = 7.8, 2.4, 1.2 Hz, 1H), 3.90 (s, 3H), 3.48 (s, 3H). |
| 129 | (400 MHz, CDCl₃) δ 10.12 (s, 1H), 8.46 (s, 1H), 8.21 (s, 1H), 7.84 (dd, J = |
| | 8.8, 0.8 Hz, 2H), 7.70 (dd, J = 6.8, 2.0 Hz, 2H), 7.64-7.62 (m, 2H), 7.59 |
| | (ddd, J = 1.6, 1.2, 0.4 Hz, 2H), 7.55-7.50 (m, 3H), 7.43 (ddd, J = 7.6, 7.6, |
| | 0.8 Hz, 1H), 7.39 (ddd, J = 7.6, 1.6, 1.6 Hz, 1H), 7.09 (dd, J = 6.8, 2.0 Hz, |
| | 1H), 3.90 (s, 3H), 3.52 (s, 2H), 3.48 (s, 3H), 2.24 (s, 6H). |
| 130 | (400 MHz, CDCl₃) δ 10.19 (s, 1H), 8.47 (s, 1H), 8.29 (s, 1H), 7.68 (s, 1H), |
| | 7.63 (s, 1H), 7.60 (dd, J = 1.6, 1.6 Hz, 1H), 7.56-7.51 (m, 3H), 7.43-7.34 |
| | (m, 4H), 7.30-7.22 (m, 2H), 7.13 (dd, J = 6.8, 2.0 Hz, 1H), 3.92 (s, 3H), |
| | 3.53 (s, 2H), 3.47 (s, 3H), 2.86-2.75 (m, 4H), 2.67-2.56 (m, 2H). |
| 131 | (400 MHz, CDCl₃) δ 10.22 (s, 1H), 8.47 (s, 1H), 8.10 (s, 1H), 7.70 (dd, J = |
| | 6.8, 2.4 Hz, 2H), 7.63-7.62 (m, 2H), 7.56 (dd, J = 6.4, 2.0 Hz, 2H), 7.42- |
| | 7.35 (m, 4H), 7.30-7.24 (m, 3H), 7.09 (dd, J = 6.8, 2.4 Hz, 2H), 3.90 (s, |
| | 3H), 3.50 (s, 2H), 3.45 (s, 3H), 2.29 (s, 6H). |
| 132 | (400 MHz, DMSO-d₆) δ 10.28 (s, 1H), 8.65 (s, 1H), 8.45 (s, 1H), 8.05 (s, |
| | 1H), 7.68-7.60 (m, 3H), 7.52 (d, J = 0.6 Hz, 1H), 7.50-7.32 (m, 3H), 7.23- |
| | 7.15 (m, 2H), 7.13 (t, J = 7.6 Hz, 1H), 6.81 (t, J = 2 Hz, 1H), 6.75 (d, J = 7.7 |
| | Hz, 1H), 6.61 (dd, J= 8.0, 1.4 Hz, 1H), 5.21 (s, 2H), 3.87 (s, 3H), 3.51 (s, |
| | 3H). |
| 133 | (400 MHz, DMSO-d₆) δ 10.27 (s, 1H), 8.65 (s, 1H), 8.47 (s, 1H), 8.04 (s, |
| | 1H), 7.68-7.50 (m, 6H), 7.49-7.32 (m, 5H), 7.18-7.13 (m, 2H), 3.85 (s, |
| | 3H), 3.51 (s, 3H), 3.41 (s, 2H), 2.09 (s, 6H). |
| 134 | (400 MHz, DMSO-d₆) δ 12.44 (s, 1H), 10.34 (s, 1H), 8.61 (s, 1H), 8.31 (s, |
| | 1H), 7.74 (s, 1H), 7.71 (d, J = 2.4 Hz, 1H), 7.66-7.62 (m, 3H), 7.59-7.56 |
| | (m, 2H), 7.47-7.43 (m, 2H), 7.34 (t, J = 7.6 Hz, 1H), 7.22 (d, J = 9.2 Hz, |
| | 2H), 7.18 (d, J = 7.6 Hz, 1H), 3.47 (d, J = 0.4 Hz, 3H), 3.42 (s, 2H), 2.12 (s, |
| | 6H). |
| 135 | (400 MHz, CDCl₃) δ 10.32 (s, 1H), 8.46 (s, 1H), 8.07 (s, 1H), 7.71-7.69 (m, |
| | 3H), 7.61-7.60 (m, 1H), 7.38-7.35 (m, 2H), 7.26-7.21 (m, 3H), 7.10-7.08 |
| | (m, 2H), 6.96 (ddd, J = 7.8, 1.5, 0.8 Hz, 1H), 6.89 (ddd, J = 2.2, 1.5, 0.4 Hz, |
| | 1H), 6.72 (ddd, J = 8.0, 2.4, 0.8 Hz, 1H), 3.90 (s, 3H), 3.72 (s, 2H), 3.44 (s, |
| | 3H), 2.44 (s, 3H). |
| 136 | (400 MHz, CDCl₃) δ 8.56 (s, 1H), 8.46 (s, 1H), 8.35 (s, 1H), 7.71 (d, J = 0.8 |
| | Hz, 1H), 7.67 (dd, J = 6.8, 2.4 Hz, 2H), 7.62 (d, J = 0.4 Hz, 1H), 7.53 (dd, |
| | J = 6.8, 2.0 Hz, 2H), 7.28-7.23 (m, 3H), 7.13 (dd, J = 6.8, 2.0 Hz, 2H), 6.98 |
| | (ddd, J = 7.6, 1.6, 0.8 Hz, 1H), 6.90 (ddd, J = 2.4, 1.6, 0.4 Hz, 1H), 6.74 |
| | (ddd, J = 8.0, 2.4, 0.8 Hz, 1H), 4.20 (s, 3H), 3.90 (s, 3H), 2.39 (s, 3H). |
| 137 | (400 MHz, CDCl₃) δ 10.33 (s, 1H), 8.47 (d, J = 0.6 Hz, 1H), 8.07 (s, 1H), |
| | 7.70 (dd, J = 6.8 Hz, J = 2.0 Hz, 2H), 7.63 (s, 2H), 7.58 (d, J = 1.8 Hz, 1H), |
| | 7.52 (dt, J = 7.3, 1.8 Hz, 1H), 7.45-7.40 (m, 2H), 7.36 (d, J = 8.0 Hz, 2H), |
| | 7.27-7.24 (m, 2H), 7.07(dd, J = 6.8 Hz, J = 2.0 Hz, 2H), 3.90 (s, 3H), 3.54 |
| | (s, 2H), 3.44 (s, 3H), 2.44 (s, 3H), 2.24 (s, 6H). |
| 138 | (400 MHz, CDCl₃) δ 8.56 (s, 1H), 8.47 (s, 1H), 8.35 (s, 1H), 7.68-7.64 (m, |
| | 4H), 7.59 (d, J = 1.8 Hz, 1H), 7.54-7.50 (m, 3H), 7.43 (t, J = 7.4 Hz, 1H), |
| | 7.37 (d, J = 7.2 Hz, 1H), 7.27-7.25 (m, 2H), 7.11 (dd, J = 6.8 Hz, 2.0 Hz, |
| | 2H), 4.20 (s, 3H), 3.90 (s, 3H), 3.48 (s, 2H), 2.39 (s, 3H), 2.22 (s, 6H). |
| 139 | (400 MHz, DMSO-d₆) δ 10.38 (s, 1H), 8.62 (s, 1H), 8.49 (s, 1H), 8.07 (s, |
| | 1H), 8.00-7.94 (m, 1H), 7.72-7.65 (m, 2H), 7.61-7.54 (m, 2H), 7.50-7.41 |
| | (m, 3H), 7.36-7.27 (m, 3H), 3.47 (s, 3H), 3.44 (s, 2H), 2.11 (s, 6H). |
| 140 | (400 MHz, CDCl₃) δ 10.22 (s, 1H), 8.46 (s, 1H), 8.10 (s, 1H), 7.73-7.67 (m, |
| | 2H), 7.64 (s, 1H), 7.61 (d, J = 0.7 Hz, 1H), 7.57-7.53 (m, 2H), 7.43-7.34 |
| | (m, 4H), 7.30-7.24 (m, 1H), 7.11-7.06 (m, 2H), 3.91 (s, 3H), 3.58 (s, 2H), |
| | 3.45 (s, 3H), 2.82-2.20 (m, 11H). |
| 141 | (400 MHz, CDCl₃) δ 10.20 (s, 1H), 8.48 (s, 1H), 8.09 (s, 1H), 7.68-7.66 (m, |
| | 3H), 7.51 (s, 1H), 7.38-7.35 (m, 2H), 7.29-7.22 (m, 4H), 7.07 (d, J = 8.8 |
| | Hz, 2H), 6.86-6.81 (m, 2H), 3.88 (s, 3H), 3.73 (s, 2H), 3.44 (s, 3H). |
| 175 | (400 MHz, CDCl₃) δ 11.87 (s, 1H), 8.75 (dd, J = 7.3, 2.2 Hz, 1H), 8.47 (s, |
| | 1H), 7.96 (s, 1H), 7.91 (d, J = 0.8 Hz, 1H), 7.78 (d, J = 9.0 Hz, 2H), 7.61 |
| | (dd, J = 6.7, 2.2 Hz, 1H), 7.55-7.47 (m, 3H), 7.43-7.39 (m, 2H), 7.30-7.27 |
| | (m, 2H), 7.11 (d, J = 9.0 Hz, 2H), 6.61 (dd, J = 7.3, 6.7 Hz, 1H), 3.92 (s, |
| | 3H), 3.63 (d, J = 5.9 Hz, 2H), 2.58-2.51 (m, 2H), 1.91 (d, J = 3.3 Hz, 4H). |
| 176 | (400 MHz, CDCl₃) δ 10.24 (s, 1H), 8.47 (s, 1H), 8.10 (d, J = 0.5 Hz, 1H), |
| | 7.96 (dd, J = 0.4 Hz, 1H), 7.92 (d, J = 0.8 Hz, 1H), 7.74-7.69 (m, 2H), |
| | 7.55-7.47 (m, 4H), 7.39-7.35 (m, 2H), 7.30-7.26 (m, 3H), 7.26-7.24 (m, |
| | 1H), 7.10 (d, J = 8.9 Hz, 2H), 3.93-3.90 (m, 3H), 3.77-3.73 (m, 1H), 3.65- |
| | 3.60 (m, 2H), 3.45 (s, 3H), 2.58-2.52 (m, 2H), 1.95-1.82 (m, 6H). |
| 177 | (400 MHz, DMSO-d₆) δ 11.96 (s, 1H), 8.58 (dd, J = 7.2, 2.0 Hz, 1H), 8.46 |
| | (s, 1H), 8.11 (dd, J = 7.2, 2.0 Hz, 1H), 8.06 (s, 1H), 7.73 (d, J = 8.8 Hz, 2H), |
| | 7.63-7.59 (m, 2H), 7.53 (s, 1H), 7.44-7.40 (m, 2H), 7.20-7.15 (m, 3H), |
| | 6.81-6.80 (m, 1H), 6.80-6.76 (m, 1H), 6.72 (t, J = 7.2 Hz, 1H), 6.63-6.60 |
| | (m, 1H), 5.67 (t, J = 5.2 Hz, 1H), 3.86 (s, 3H), 3.04-2.98 (m, 2H), 1.11 (t, |
| | J = 7.2 Hz, 3H). |
| 178 | (400 MHz, CDCl₃) δ 10.21 (s, 1H), 8.46 (s, 1H), 8.09 (s, 1H), 7.72-7.69 (m, |
| | 3H), 7.63 (s, 1H), 7.39-7.35 (m, 2H), 7.31-7.21 (m, 3H), 7.09 (d, J = 8.8 |
| | Hz, 2H), 6.91-6.88 (m, 1H), 6.82-6.81 (m, 1H), 6.65-6.62 (m, 1H), 3.89 (s, |
| | 3H), 3.66 (s, 1H), 3.44 (s, 3H), 3.14 (q, J = 7.2 Hz, 2H), 1.23 (t, J = 7.2 Hz, |
| | 3H). |

### AXL and MERTK Inhibiting Activities

### In Vitro Activities

Exemplary compounds of formula (I) thus prepared were evaluated for their in vitro efficacy in inhibiting AXL and MERTK proteins.

A purified kinase (AXL or MERTK) was incubated with a compound or DMSO (control) for 15 minutes in an assay buffer (25 mM Tri s pH 7.4, 10 mM MgCl₂, 4 mM MnCl₂, 2 mM DTT, 0.0100 BSA, 0.02% TritonX-100, 0.0100 Brij35 and 0.5 mM Na₃VO₄ for MERTK; and 40 mM Tri s pH 7.4, 20 mM MgCl₂, 2 mM DTT, 0.01% BSA and 0.5 mM Na₃VO₄ for AXL). The above-prepared substrate and ATP (12 μM for MERTK; 50 μM for AXL) were added. The mixture was incubated for 3 hours at 30° C. The luminescence was calculated to determine the kinase activity using a Kinase Glo assay kit for MERTK and an ADP-Glo Kinase Assay kit for AXL following the manufacture's instructions (Promega Corp., Madison, Wisconsin).

The results are shown in Table 3 below.

TABLE 3

| Compound | MER Enzymatic | AXL Enzymatic | Dual MER/AXL |
|---|---|---|---|
| No | Inhibition (%) | Inhibition (%) | Inhibition |
| 1 | ++ | +++ | ++ |
| 2 | ++ | +++ | ++ |
| 3 | +++ | +++ | +++ |
| 4 | ++ | +++ | ++ |
| 5 | + | + | + |
| 6 | + | + | + |
| 7 | +++ | ++ | ++ |
| 8 | ++ | + | + |
| 9 | + | + | + |
| 10 | + | + | + |
| 11 | +++ | +++ | +++ |
| 12 | + | + | + |
| 13 | + | + | + |
| 14 | + | +++ | + |
| 15 | ++ | + | + |
| 16 | + | +++ | + |
| 17 | + | +++ | + |
| 18 | + | +++ | + |
| 19 | ++ | +++ | ++ |
| 20 | ++ | ++ | ++ |
| 21 | + | + | + |
| 22 | + | ++ | + |
| 23 | + | + | + |
| 24 | ++ | +++ | ++ |
| 25 | ++ | +++ | ++ |
| 26 | + | + | + |
| 27 | + | +++ | + |
| 28 | ++ | +++ | ++ |
| 29 | + | +++ | + |
| 30 | +++ | +++ | +++ |
| 31 | + | +++ | + |
| 32 | +++ | +++ | +++ |
| 33 | +++ | +++ | +++ |
| 34 | +++ | +++ | +++ |
| 35 | + | ++ | + |
| 36 | + | ++ | + |
| 37 | +++ | +++ | +++ |
| 38 | ++ | +++ | ++ |
| 39 | ++ | +++ | ++ |
| 40 | ++ | +++ | ++ |
| 41 | + | ++ | + |
| 42 | + | + | + |
| 43 | ++ | ++ | ++ |
| 44 | ++ | +++ | ++ |
| 45 | + | +++ | + |
| 46 | +++ | +++ | +++ |
| 47 | + | ++ | + |
| 48 | + | + | + |
| 49 | + | ++ | + |
| 50 | + | ++ | + |
| 51 | + | +++ | + |
| 52 | ++ | +++ | ++ |
| 53 | ++ | ++ | ++ |
| 54 | ++ | +++ | ++ |
| 55 | ++ | +++ | ++ |
| 56 | ++ | +++ | ++ |
| 57 | + | +++ | + |
| 58 | ++ | +++ | ++ |
| 59 | + | +++ | + |
| 60 | ++ | +++ | ++ |
| 61 | ++ | +++ | ++ |
| 62 | + | +++ | + |
| 63 | +++ | +++ | +++ |
| 64 | + | + | + |
| 65 | + | ++ | + |
| 66 | + | + | + |
| 67 | + | +++ | + |
| 68 | ++ | +++ | ++ |
| 69 | + | +++ | + |
| 70 | +++ | +++ | +++ |
| 71 | +++ | +++ | +++ |
| 72 | + | + | + |
| 73 | ++ | +++ | ++ |
| 74 | + | + | + |
| 75 | + | + | + |
| 76 | + | + | + |
| 77 | + | + | + |
| 78 | ++ | +++ | ++ |
| 79 | + | + | + |
| 80 | ++ | +++ | ++ |
| 81 | + | + | + |
| 82 | +++ | +++ | +++ |
| 83 | + | + | + |
| 84 | + | + | + |
| 85 | + | +++ | + |
| 86 | + | + | + |
| 87 | +++ | +++ | +++ |
| 88 | ++ | +++ | ++ |
| 89 | +++ | +++ | +++ |
| 90 | ++ | +++ | ++ |
| 91 | + | + | + |
| 92 | ++ | +++ | ++ |
| 93 | ++ | +++ | ++ |
| 94 | ++ | +++ | ++ |
| 95 | +++ | +++ | +++ |
| 96 | ++ | +++ | ++ |
| 97 | ++ | +++ | ++ |
| 98 | ++ | +++ | ++ |
| 113 | ++ | +++ | ++ |
| 114 | ++ | +++ | ++ |
| 115 | + | + | + |
| 116 | + | + | + |
| 117 | + | ++ | + |
| 118 | + | + | + |
| 119 | + | ++ | + |
| 120 | + | + | + |
| 121 | +++ | +++ | +++ |
| 122 | + | +++ | + |
| 123 | ++ | +++ | ++ |
| 124 | + | + | + |
| 125 | + | + | + |
| 126 | +++ | +++ | +++ |
| 127 | + | +++ | + |
| 128 | + | +++ | + |
| 129 | + | +++ | + |
| 130 | + | + | + |
| 131 | ++ | +++ | ++ |
| 132 | + | +++ | + |
| 133 | ++ | +++ | ++ |
| 134 | + | + | + |
| 135 | + | +++ | + |
| 136 | + | + | + |
| 137 | + | +++ | + |
| 138 | + | + | + |
| 139 | + | + | + |
| 140 | +++ | +++ | +++ |
| 141 | + | + | + |
| 175 | + | + | + |
| 176 | + | + | + |
| 177 | + | + | + |
| 178 | + | +++ | + |

Note that in Table 2 above, “+++” indicates an IC₅₀ value less than 20 nM; “++” indicates an IC₅₀ value between 20 nM and 100 nM; and “+” indicates an IC₅₀ value greater than 100 nM but less than 50,000 nM.

### In Vitro Enzymatic Assay

A study was conducted to compare exemplary compounds of formula (I) in inhibiting TAM activities, including MERTK, AXL, and TYRO3. An enzymatic assay was used in the study. A purified Kinase (i.e., MERTK, AXL, or TYRO3) was incubated with a compound or DMSO (control) for around 15 minutes in an assay buffer (25 mM Tris pH 7.4, 10 mM MgCl₂, 4 mM MnCl₂, 2 mM DTT, 0.01% BSA, 0.02% TritonX-100, 0.01% Brij35 and 0.5 mM Na₃VO₄ for MERTK; 40 mM Tris pH 7.4, 20 mM MgCl₂, 2 mM DTT, 0.01% BSA and 0.5 mM Na₃VO₄ for AXL; and 25 mM Tris pH 7.4, 10 mM MgCl₂, 4 mM MnCl₂, 2 mM DTT, 0.01% BSA and 0.5 mM Na₃VO₄ for TYRO3). The substrate thus obtained and ATP (12 μM for MERTK; 50 μM for AXL and TYRO3) were added. The mixture was incubated for 3 hours at 30° C. The luminescence was calculated to determine the kinase activity using a Kinase Glo assay kit for MERTK and an ADP-Glo Kinase assay kit for AXL and TYRO3 following the manufacture's instructions (Promega). Inhibition rates and half maximal inhibitory concentration (IC₅₀) values were calculated. High percentage inhibition rates and low IC₅₀ values are indicatives of high potency of the test compound.

Eight compounds of this invention (i.e., compounds 3, 4, 7, 11, 37, 46, 114, and 123) each were tested against MERTK, AXL, and TYRO3. Their results are shown in Table 4 below.

TABLE 4

| | IC₅₀ (nM) | | |
|---|---|---|---|
| Compound | MERTK | AXL | TYRO3 |
| 3 | 11.6 | 12.9 | 18.3 |
| 4 | 22.9 | 10.5 | 15.6 |
| 7 | 12.0 | 80.3 | 53.5 |
| 11 | 10.4 | 12.2 | 34 |
| 37 | 2.7 | 7.2 | 24.5 |
| 46 | 15.7 | 9.1 | 46.1 |
| 114 | 25.3 | 11.9 | 637.6 |
| 123 | 21.5 | 9.8 | 991.3 |

### In Vitro Ba/F3-MERTK Cellular Assay

Compounds 11, 37, 114, and 123 were assessed for their in vivo efficacy in inhibiting MERTK using a Ba/F3-MERTK cellular assay.

Cells were seeded in 96-well plates at an opportune density. Then, cells were treated with a compound for 72 hours. At the end of the treatment, for a 96-well microtiter plate, an MTS reaction medium were prepared using phenol red-free DMEM, MTS (tetrazolium compound [3-(4,5-dimethylthiozol-2-yl)-5-(3-carboxymethoxyphenyl)-2-(4-sulfophenyl)-2H-tetrazolium, inner salt]; Promega, Madison, WI) and PMS (phenazine methosulfate; Sigma, St. Louis, MO) in a ratio of 8:2:0.1, respectively. The MTS reaction medium was distributed to cells (100 μL/well), which were incubated for 1.5 hours at 37° C. in a humidified, 5% CO2 atmosphere. The absorbance was recorded at 490 nm.

The results showed that these four exemplary compounds are all potent inhibitors, each exhibiting an IC₅₀ value in the range of 4.2 nM to 138.7 nM.

### Immune Modulatory Activity

Compound 11 was evaluated for its immune modulatory activity in an MC38 murine colon tumor model. Female C57BL/6J mice between 8-10 weeks of age were used. Murine colon tumor MC38 cells were detected as free of Mycoplasma spp prior to injection into animals. MC38 cells at 10⁵ cells per mouse in 100 μL culture medium were implanted subcutaneously into the left flank region of mice with a 25⅝-gauge needle (n=5-6 animals per group). Compound 11 was dissolved in a mixture of 10% DMA: 40% PEG400:50% (1% CMC) (v/v/v). Treatments were initiated after randomization with inclusion of tumors at approximately 150 mm³. Tumor growths were measured with an electronic caliper and volumes are calculated as L×W×W/2. Tumor size and animal body weight were measured twice a week after tumor cell inoculation. All experiments were conducted in accordance with the protocols approved by National Health Research Institutes' Institutional Animal Care and Use Committee.

A group of mice received orally either vehicle only or Compound 11 at 50 mg/kg, twice per day for five days. Ten days after the treatment, the tumor cells and spleen cells in the mice were collected to study effect of this compound on the immune cells, i.e., tumor-associated macrophages and T cells. Briefly, tumor and spleen from vehicle control and Compound 11 were harvested and isolated into single cells. Single cell suspension were pre-incubated with mouse Fc receptor blocker before staining with appropriate antibody conjugate for Cd45, Cd3, Cd4, Cd8, F4/80, Cd11b, Cd86, AXL, MERTK and analyzed by flow cytometry to quantify the accumulation of immune cells including Cd4 (Cd3⁺Cd4⁺), Cd8 (Cd3⁺Cd8⁺), macrophages (Cd11b⁺F4/80⁺), tumor associated macrophage (M2, Cd11b⁺F4/80⁺Cd206⁺), classical macrophage (M1, Cd11b⁺F4/80+/Cd86⁺) populations. For intracellular staining of analysis of tumor-associated macrophage marker Cd206 expression, cells were fixed by using Cytofix/Cytoperm™ kit (BD Biosciences, Franklin Lakes, New Jersey) after surface marker staining and then stain with anti-mouse Cd206 antibody conjugate. Live/dead fixable dye were used to exclude non-viable cells from the analyses.

The results showed that, (i) 10 days after the treatment, the tumor size in the mice treated with Compound 11 increased slightly from 200 mm³ to 350 mm³ and, by contrast, the tumor size in the mice treated with vehicle increased significantly from 200 mm³ to 1000 mm³, (ii) in the tumor cells, the level of macrophages decreased from 18% to 13% and the level of its M2-phenotype decreased from 10% to 5%, (iii) in the spleen cells, the levels of total T cells, CD4⁺ T cells, and CD8⁺ T cells increased, respectively, from 30% to 43%, from 5% to 10%, and from 18% to 28%; and (iv) also in the spleen cells, expressions of AXL in CD8⁺ T cells and CD4⁺ T cells decreased respectively from 13% to 5% and from 1.8% to 0.7%, and expressions of MERTK in CD8⁺ T cells and CD4⁺ T cells decreased respectively from 32% to 20% and from 7% to 3%.

### In Vivo Activity Against Colon Tumor

Compound 11 was evaluated for its in vivo efficacy in treating colon tumor. Female C57BL/6J mice between 8-10 weeks of age were used. Murine colon tumor MC38 cells were detected as free of Mycoplasma spp prior to injection into animals. MC38 cells at 10⁵ cells per mouse in 100 μL culture medium were implanted subcutaneously into the left flank region of mice with a 25⅝-gauge needle (n=8 animals per group). Compound 11 was dissolved in 10% DMA: 40% PEG400:50% (1% CMC) (v/v/v). Treatments were initiated after randomization with inclusion of tumors at approximately 50 mm³.

Compound 11 was orally administered at 50 mg/kg twice a day for 5 days to a group of mice bearing MC-38 murine colon tumor. Control animals were orally administered with vehicle only. Tumor growths were measured with an electronic caliper and volumes were calculated as L×W×W/2. Tumor size and animal body weight were measured twice a week after tumor cell inoculation. After the treatment, tumor volumes and body weight changes were measured.

The results showed that, at day 17 after the treatment, the tumor volume in the group treated with Compound 11 increased from 0 to 300 mm³. By contrast, the tumor volume in the control group grew from 0 to 2000 mm³.

### In Vivo Activity Against Breast Cancer

Compound 11 was further evaluated for its in vivo efficacy in treating breast cancer. MDA-MB-231 TNBC cell lines were used. Prior to injection into animals, the cancer cells were detected as free of Mycoplasma spp. Five million MDA-MB-231 cells in culture medium and matrigel (1:1, v/v) were injected subcutaneously into the left flank region of the NOD/SCID mice (n=8 animals per group). Treatments were initiated after randomization with inclusion of tumors at approximately 150 mm³. The compound was prepared using 10% DMA: 40% PEG400:50% (1% CMC) (v/v/v).

Three groups of mice were used, i.e., (1) a control group, (2) a group treated with Compound 11 at 25 mg/kg, and (3) a group treated with Compound 11 at 50 mg/kg. Each group was administered orally twice a day for five days. Control animals were orally administered with vehicle only. Tumor growths were measured with an electronic caliper and volumes were calculated as L×W×W/2. Tumor size and animal body weight were measured twice a week after tumor cell inoculation.

The results showed that at day 35 after the treatment, tumor volumes in groups (1)-(3) increased from 150 mm³ to, respectively, 600 mm³, 300 mm³, and 220 mm³.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. For example, compounds structurally analogous to the compounds of this invention also can be made, screened for their efficacy in treating a condition that relates to SOS1. Thus, other embodiments are also within the claims.

1. A compound of Formula (I): 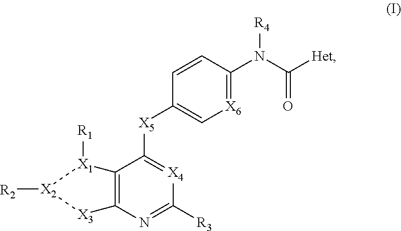
in which
each of R₁ and R₂, independently, is H, deuterium, halo, C₁₋₆ alkyl, C₃₋₆ cycloalkyl, C₁₋₆ heterocycloalkyl, aryl, heteroaryl, or deleted;
R₃ is H, halo, C₁₋₆ alkyl, C₃₋₆ cycloalkyl, C₁₋₆ heterocycloalkyl, aryl, heteroaryl, or —NR₃ₐR_{3b}, each of R₃ₐ and R_{3b} being, independently, H, C₁₋₆ alkyl, or aryl;
R₄ is H, C₁₋₆ alkyl, C₃₋₆ cycloalkyl, or C₁₋₆ heterocycloalkyl;
X₁ is C, CH, CD, or S;
X₂ is C or N;
X₃ is O, CH, S, or NH;
at least one of X₁, X₂, and X₃ is O, S, N, or NH;
X₄ is N, CH, or CH(CN);
X₅ is O or NH;
X₆ is N or CH;
Het is selected from the group consisting of: 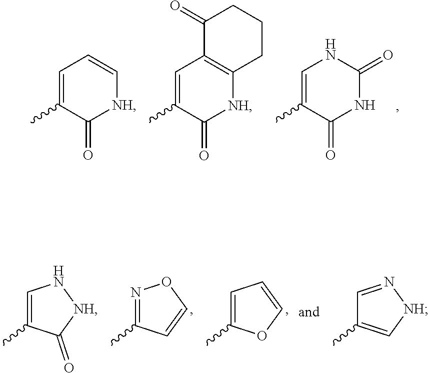
one of the two [symbol] is a single bond and the other is a double bond; and
each of C₁₋₆ alkyl, C₃₋₆ cycloalkyl, C₁₋₆ heterocycloalkyl, aryl, heteroaryl, and Het is unsubstituted or substituted with one or more of the chemical groups selected from the group consisting of deuterium, hydroxyl, halo, nitro, cyano, amino, C₁₋₆ acylamino, C₃₋₆ alkylamino, C₁₋₆ alkyl, C₁₋₆ aminoalkyl, C₁₋₆ alkoxyl, C₁₋₆ alkylcarbonyl, C₃₋₁₀ cycloalkyl, C₁₋₆ heterocycloalkyl, aralkyl, aryl, and heteroaryl.

2. The compound of claim 1, wherein each of X₁ and X₂ is C, X₃ is O or NH, X₄ is N, and the [symbol] between X₁ and X₂ is a double bond.

3. The compound of claim 1, wherein R₁ is H or phenyl.

4. The compound of claim 1, wherein R₂ is H, C₁₋₆ alkyl, or heteroaryl.

5. The compound of claim 1, wherein R₃ is H, methyl, phenyl, pyridinyl, methylamino, or phenylamino, and R₄ is H.

6. The compound of claim 1, wherein X₅ is O and X₆ is CH.

7. The compound of claim 1, wherein Het is 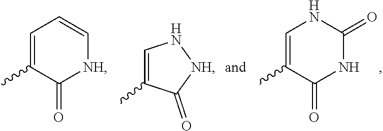
unsubstituted or substituted with one or more of methyl, ethoxy, phenyl, 3-fluorophenyl, 4-fluorophenyl, 2-methyl-4-fluorophenyl, 2-fluoropyridin-3-yl, and butylenecarbonyl.

8. The compound of claim 1, wherein the compound is a compound of formula (II),

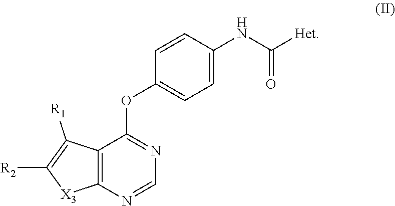

9. The compound of claim 8, wherein X₃ is O or NH.

10. The compound of claim 8, wherein Het is 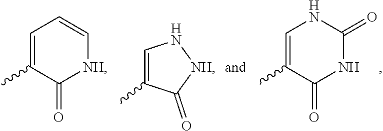
each of which is unsubstituted or substituted with one or more of C₁₋₆ alkyl and phenyl, phenyl being unsubstituted or substituted with hydroxyl, halo, amino, C₁₋₆ alkyl, or C₁₋₆ alkoxy.

11. The compound of claim 8, wherein R₁ is phenyl and R₂ is H or heteroaryl, each of phenyl and heteroaryl is unsubstituted or substituted with one or more of the chemical groups consisting of amino, C₁₋₆ acylamino, C₁₋₆ alkylamino, C₁₋₆ alkyl, and C₁₋₆ aminoalkyl.

12. The compound of claim 1, wherein the compound is one of Compounds 1-174.

13. The compound of claim 12, wherein the compound is one of compounds 3, 11, 30, 37, 46, 63, 71, 82, 95, 121, and 123.

14. A method of treating a disorder comprising administering to a subject in need thereof an effective amount of a compound of claim 1, wherein the disorder is associated with TAM.

15. A pharmaceutical composition comprising a compound of claim 1 and a pharmaceutically acceptable carrier thereof.
